(19) United States
(12) Patent Application Publication
(10) Pub. No.: US 2020/0399588 A1
(43) Pub. Date: Dec. 24, 2020

(54) WNT COMPOSITIONS AND METHODS OF PROCESS FROM SERUM-FREE CULTURING CONDITIONS

(71) Applicant: Ankasa Regenerative Therapeutics, Inc., La Jolla, CA (US)

(72) Inventors: Ping Yuan, JR., Newark, CA (US); Jill Helms, Portola Valley, CA (US); Ying Zhu, Palo Alto, CA (US); Bo Liu, Palo Alto, CA (US); Stefanie Gaster, La Jolla, CA (US)

(21) Appl. No.: 16/996,676

(22) Filed: Aug. 18, 2020

Related U.S. Application Data
(63) Continuation of application No. PCT/US18/44886, filed on Aug. 1, 2018.
(60) Provisional application No. 62/539,960, filed on Aug. 1, 2017.
(60) Provisional application No. 62/630,448, filed on Feb. 14, 2018.

(51) Int. Cl. C12N 5/00; C07K 1/18; A61K 47/26; A61K 47/69; C12N 5/071

(52) U.S. Cl. CPC C12N 5/0031; C07K 1/18; C12N 2501/415; A61K 47/6911; C12N 5/0602; A61K 47/26

(57) ABSTRACT

Disclosed herein are methods and compositions for producing a Wnt polypeptide under a serum-free condition. Also disclosed herein are methods of purifying the Wnt polypeptide from a serum-free condition.

### CROSS-REFERENCE

This application is a continuation of Application No. PCT/US18/44886, filed on Aug. 1, 2018, which claims the benefit of U.S. Provisional Application No. 62/539,960, filed on Aug. 1, 2017, and U.S. Provisional Application No. 62/630,448, filed on Feb. 14, 2018, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 30, 2018, is named 47271-708_301_SL.txt and is 63,855 bytes in size.

### BACKGROUND OF THE DISCLOSURE

Wnt proteins form a family of highly conserved secreted signaling molecules that bind to cell surface receptors encoded by the Frizzled and low-density lipoprotein receptor related proteins (LRPs). The WNT gene family consists of structurally related genes which encode secreted signaling proteins. These proteins have been implicated in oncogenesis and in several developmental processes, including regulation of cell fate and patterning during embryogenesis. Once bound, the ligands initiate a cascade of intracellular events that eventually lead to the transcription of target genes through the nuclear activity of β-catenin and the DNA binding protein TCF.

### SUMMARY OF THE DISCLOSURE

Disclosed herein, in certain embodiments, are Wnt compositions and methods of producing Wnt from a serum-free condition. In some embodiments, the Wnt composition comprises a Wnt3A composition. In some embodiments, described herein comprise methods of producing Wnt3A from a serum-free condition.

Disclosed herein, in certain embodiments, is a method of preparing a functionally active Wnt polypeptide, comprising: (a) incubating a plurality of Wnt polypeptide-chaperone complexes with a buffer comprising a sugar detergent to generate a mixture comprising a first Wnt composition comprising a functionally inactive Wnt polypeptide and a chaperone composition; (b) separating the first Wnt composition from the mixture with a column immobilized with a sulfonated polyaromatic compound to generate a second Wnt composition comprising the functionally active Wnt polypeptide and the sugar detergent; (c) optionally purifying the second Wnt composition with an affinity chromatography column comprising a polypeptide that interacts with the Fc portion of an antibody, a mixed mode column, a size exclusion chromatography column, or a combination thereof, at least once to generate a third Wnt composition; and (d) contacting the second Wnt composition or optionally the third Wnt composition with an aqueous solution of liposomes to generate a final Wnt composition comprising a functionally active Wnt polypeptide. In some embodiments, the sugar detergent comprises a glucoside detergent. In some embodiments, the glucoside detergent is n-hexyl-β-D-glucopyranoside, n-heptyl-β-D-glucopyranoside, n-octyl-β-D-glucopyranoside, n-octyl-α-D-glucopyranoside, octyl β-D-1-thioglucopyranoside, n-octyl-β-D-galactopyranoside, n-nonyl-β-D-glucopyranoside, n-decyl-β-D-glucopyranoside, n-dodecyl-β-D-glucopyranoside, or methyl-6-O—(N-heptylcarbamoyl)-α-D-glucopyranoside. In some embodiments, the glucoside detergent is selected from n-octyl-β-D-glucopyranoside and octyl β-D-1-thioglucopyranoside. In some embodiments, the glucoside detergent is n-octyl-β-D-glucopyranoside. In some embodiments, the glucoside detergent is octyl β-D-1-thioglucopyranoside. In some embodiments, the sugar detergent comprises a maltoside detergent. In some embodiments, the maltoside detergent is n-decyl-β-D-maltopyranoside, n-dodecyl-β-D-maltopyranoside, or 6-cyclohexyl-1-hexyl-β-D-maltopyranoside. In some embodiments, the concentration of the sugar detergent in the buffer is from about 0.1% to about 5% w/v. In some embodiments, the concentration of the sugar detergent in the buffer is about 0.1%, 0.5%, 1%, 1.5%, or about 2% w/v. In some embodiments, the plurality of Wnt polypeptide-chaperone complexes is further purified with an affinity chromatography column comprising a polypeptide that interacts with the Fc portion of an antibody prior to incubating with the buffer to generate the mixture comprising the first Wnt composition. In some embodiments, the affinity chromatography column is a Protein A column. In some embodiments, the plurality of Wnt polypeptide-chaperone complexes is eluted from the affinity chromatography column with a buffer comprising a pH of less than 5, less than 4, or less than 3. In some embodiments, the method comprises: (a) purifying the plurality of Wnt polypeptide-chaperone complexes on a first affinity chromatography column comprising a polypeptide that interacts with the Fc portion of an antibody to generate an eluted mixture of Wnt polypeptide-chaperone complexes; (b) incubating the eluted mixture of Wnt polypeptide-chaperone complexes with the buffer comprising a sugar detergent to generate the mixture comprising the first Wnt composition comprising a functionally inactive Wnt polypeptide and a chaperone composition; (c) separating the first Wnt composition from the mixture with a column immobilized with a sulfonated polyaromatic compound to generate the second Wnt composition comprising the functionally active Wnt polypeptide and the sugar detergent; (d) purifying the second Wnt composition in tandem with a second affinity chromatography column comprising a polypeptide that interacts with the Fc portion of an antibody, a mixed mode column, and a size exclusion chromatography column to generate the third Wnt composition; and (e) contacting the third Wnt composition with an aqueous solution of liposomes to generate the final Wnt composition comprising a functionally active Wnt polypeptide. In some embodiments, the first affinity chromatography column and the second affinity chromatography column are each independently a Protein A column. In some embodiments, an elution buffer for the mixed mode column comprises from about 0.1M to about 2M, from about 0.1M to about 1M, or from about 0.1M to about 0.5M arginine. In some embodiments, an elution buffer for each of the second affinity chromatography column, the mixed mode column, and the size exclusion chromatography column comprises the sugar detergent. In some embodiments, the separating of step b) comprises eluting the first Wnt composition with a step gradient comprising a first buffer solution at a first salt concentration and a second buffer solution at a second salt concentration. In some embodiments, the first buffer solution comprises a salt at a concentration of from about 10 mM to about 100 mM. In some embodiments, the first buffer solution comprises a salt at a concentration of about 10 mM, 20 mM, 30 mM, 40 mM, 50 mM, or higher. In some embodiments, the second buffer solution comprises a salt at a concentration of about 1M, 1.5M, 2M, or higher. In some embodiments, the salt comprises sodium chloride, potassium chloride, magnesium chloride, calcium chloride, calcium phosphate, potassium phosphate, magnesium phosphate, sodium phosphate, ammonium sulfate, ammonium chloride, or ammonium phosphate. In some embodiments, the chaperone comprises a Frizzled protein. In some embodiments, the chaperone comprises Wntless. In some embodiments, the chaperone comprises Afamin. In some embodiments, the chaperone comprises a Frizzled-8 fusion protein. In some embodiments, the Frizzled-8 fusion protein comprises a truncated Frizzled-8 protein. In some embodiments, the truncated Frizzled-8 protein comprises a cysteine-rich region (CRD) of Frizzled-8. In some embodiments, the truncated Frizzled-8 protein comprises the region spanning amino acid residue 25 to amino acid residue 172 of SEQ ID NO: 4. In some embodiments, the Frizzled-8 fusion protein further comprises an IgG Fc portion. In some embodiments, the Frizzled-8 fusion protein comprises at least 80%, 85%, 90%, 95%, 96%, 97%, 98%, or 99% sequence identity to SEQ ID NO: 5. In some embodiments, the Frizzled-8 fusion protein comprises at least 80%, 85%, 90%, 95%, 96%, 97%, 98%, or 99% sequence identity to SEQ ID NO: 18. In some embodiments, the Wnt polypeptide comprises a heterologous signal sequence. In some embodiments, the Wnt polypeptide comprises a native signal sequence. In some embodiments, the Wnt polypeptide comprises a tag. In some embodiments, the tag comprises a HIS(6×)-tag (SEQ ID NO: 19), a FLAG tag, or a PA tag. In some embodiments, the Wnt polypeptide is a Wnt3A polypeptide, a Wnt5B polypeptide, or a Wnt10B polypeptide. In some embodiments, the Wnt polypeptide is a Wnt3A polypeptide. In some embodiments, the Wnt3A polypeptide is polypeptide that comprises about 90%, 95%, 99%, or more sequence identity to SEQ ID NO: 1. In some embodiments, the Wnt3A polypeptide comprises a truncation of about 1 to about 33 amino acids. In some embodiments, the truncation is a C-terminal truncation. In some embodiments, the Wnt3A polypeptide is a polypeptide of SEQ ID NO: 1 with a C-terminal truncation. In some embodiments, the Wnt3A polypeptide comprises about 90%, 95%, 99%, or more sequence identity to SEQ ID NO: 2. In some embodiments, the Wnt3A polypeptide consists of SEQ ID NO: 2. In some embodiments, the Wnt polypeptide comprises a lipid modification at an amino acid position corresponding to amino acid residue 209 as set forth in SEQ ID NO: 1. In some embodiments, the Wnt polypeptide is modified with palmitic acid. In some embodiments, the second affinity chromatography column removes residual Frizzled-8 fusion proteins from the second Wnt composition. In some embodiments, the mixed mode column removes Wnt polypeptide fragments from the second Wnt composition. In some embodiments, the size exclusion chromatography column removes residual Wnt polypeptide fragments from the second Wnt composition to generate the third Wnt composition. In some embodiments, the second Wnt composition is greater than 60%, 65%, 70%, 75%, 80%, 85%, 90%, 95%, 96%, 97%, 98%, or 99% pure, relative to an equivalent Wnt composition that is purified in the absence of the sugar detergent. In some embodiments, the third Wnt composition is greater than 60%, 65%, 70%, 75%, 80%, 85%, 90%, 95%, 96%, 97%, 98%, or 99% pure, relative to an equivalent Wnt composition that is purified in the absence of the sugar detergent. In some embodiments, the final Wnt composition has a liposomal particle size distribution of from about 10 nm to about 1 μm, from 10 nm to about 500 nm, from about 50 nm to about 300 nm, from about 50 nm to about 200 nm, from about 100 nm to about 500 nm, from about 100 nm to about 300 nm, or from about 100 nm to about 200 nm. In some embodiments, the final Wnt composition has a liposomal particle size distribution of less than about 1 μm, less than about 500 nm, less than about 300 nm, less than about 200 nm, or less than about 150 nm. In some embodiments, the plurality of Wnt polypeptide-chaperone complexes is further harvested from a conditioned media comprising a cell that coexpresses Wnt polypeptides and chaperones. In some embodiments, the cell is a cGMP-compatible cell. In some embodiments, the cGMP-compatible cell is a cGMP-compatible mammalian cell, optionally selected from a Chinese Hamster Ovary (CHO) cell line, a human embryonic kidney (HEK) cell line, or a baby hamster kidney (BHK) cell line.

Disclosed herein, in certain embodiments, is a method of preparing a functionally active Wnt polypeptide, comprising: (a) co-expressing a Wnt polypeptide and a chaperone in a cell in a conditioned media to generate a plurality of Wnt polypeptide-chaperone complexes; (b) harvesting the plurality of Wnt polypeptide-chaperone complexes from the conditioned media; (c) incubating the plurality of Wnt polypeptide-chaperone complexes with a buffer comprising a sugar detergent to generate a mixture comprising a first Wnt composition comprising a functionally inactive Wnt polypeptide and a chaperone composition; (d) separating the first Wnt composition from the mixture with a column immobilized with a sulfonated polyaromatic compound to generate a second Wnt composition comprising the functionally active Wnt polypeptide and the sugar detergent; and (e) contacting the second Wnt composition with an aqueous solution of liposomes to generate a final Wnt composition comprising a functionally active Wnt polypeptide. In some embodiments, the sugar detergent comprises a glucoside detergent. In some embodiments, the glucoside detergent is n-hexyl-β-D-glucopyranoside, n-heptyl-β-D-glucopyranoside, n-octyl-β-D-glucopyranoside, n-octyl-α-D-glucopyranoside, octyl β-D-1-thioglucopyranoside, n-octyl-β-D-galactopyranoside, n-nonyl-β-D-glucopyranoside, n-decyl-β-D-glucopyranoside, n-dodecyl-β-D-glucopyranoside, or methyl-6-O—(N-heptylcarbamoyl)-α-D-glucopyranoside. In some embodiments, the glucoside detergent is selected from n-octyl-β-D-glucopyranoside and octyl β-D-1-thioglucopyranoside. In some embodiments, the glucoside detergent is n-octyl-β-D-glucopyranoside. In some embodiments, the glucoside detergent is octyl β-D-1-thioglucopyranoside. In some embodiments, the sugar detergent comprises a maltoside detergent. In some embodiments, the maltoside detergent is n-decyl-β-D-maltopyranoside, n-dodecyl-β-D-maltopyranoside, or 6-cyclohexyl-1-hexyl-β-D-maltopyranoside. In some embodiments, the concentration of the sugar detergent in the buffer is: from about 0.1% to about 5% w/v; or about 0.1%, 0.5%, 1%, 1.5%, or about 2% w/v. In some embodiments, the second Wnt composition is further purified with an affinity chromatography column comprising a polypeptide that interacts with the Fc portion of an antibody, a mixed mode column, a size exclusion chromatography column, or a combination thereof, at least once to generate a third Wnt composition. In some embodiments, the plurality of Wnt polypeptide-chaperone complexes is further purified with an affinity chromatography column comprising a polypeptide that interacts with the Fc portion of an antibody prior to incubating with the buffer to generate the mixture comprising the first Wnt composition. In some embodiments, the affinity chromatography column is a Protein A column. In some embodiments, the plurality of Wnt polypeptide-chaperone complexes is eluted from the affinity chromatography column with a buffer comprising a pH of less than 5, less than 4, or less than 3. In some embodiments, the method comprises: (a) purifying the plurality of Wnt polypeptide-chaperone complexes on a first affinity chromatography column comprising a polypeptide that interacts with the Fc portion of an antibody to generate an eluted mixture of Wnt polypeptide-chaperone complexes; (b) incubating the eluted mixture of Wnt polypeptide-chaperone complexes with the buffer comprising a sugar detergent to generate the mixture comprising the first Wnt composition comprising a functionally inactive Wnt polypeptide and a chaperone composition; (c) separating the first Wnt composition from the mixture with a column immobilized with a sulfonated polyaromatic compound to generate the second Wnt composition comprising the functionally active Wnt polypeptide and the sugar detergent; (d) purifying the second Wnt composition in tandem with a second affinity chromatography column comprising a polypeptide that interacts with the Fc portion of an antibody, a mixed mode column, and a size exclusion chromatography column to generate the third Wnt composition; and (e) contacting the third Wnt composition with an aqueous solution of liposomes to generate the final Wnt composition comprising a functionally active Wnt polypeptide. In some embodiments, the first affinity chromatography column and the second affinity chromatography column are each independently a Protein A column. In some embodiments, an elution buffer for the mixed mode column comprises from about 0.1M to about 2M, from about 0.1M to about 1M, or from about 0.1M to about 0.5M arginine. In some embodiments, an elution buffer for each of the second affinity chromatography column, the mixed mode column, and the size exclusion chromatography column comprises the sugar detergent. In some embodiments, the separating of step d) comprises eluting the first Wnt composition with a step gradient comprising a first buffer solution at a first salt concentration and a second buffer solution at a second salt concentration. In some embodiments, the first buffer solution comprises a salt at a concentration of: from about 10 mM to about 100 mM; or about 10 mM, 20 mM, 30 mM, 40 mM, 50 mM, or higher. In some embodiments, the second buffer solution comprises a salt at a concentration of about 1M, 1.5M, 2M, or higher. In some embodiments, the salt comprises sodium chloride, potassium chloride, magnesium chloride, calcium chloride, calcium phosphate, potassium phosphate, magnesium phosphate, sodium phosphate, ammonium sulfate, ammonium chloride, or ammonium phosphate. In some embodiments, the chaperone comprises a Frizzled protein. In some embodiments, the chaperone comprises a Frizzled-8 fusion protein. In some embodiments, the Frizzled-8 fusion protein comprises a truncated Frizzled-8 protein. In some embodiments, the truncated Frizzled-8 protein comprises a cysteine-rich region (CRD) of Frizzled-8. In some embodiments, the truncated Frizzled-8 protein comprises the region spanning amino acid residue 25 to amino acid residue 172 of SEQ ID NO: 4. In some embodiments, the Frizzled-8 fusion protein further comprises an IgG Fc portion. In some embodiments, the Frizzled-8 fusion protein comprises: at least 80%, 85%, 90%, 95%, 96%, 97%, 98%, or 99% sequence identity to SEQ ID NO: 5; or at least 80%, 85%, 90%, 95%, 96%, 97%, 98%, or 99% sequence identity to SEQ ID NO: 18. In some embodiments, the Wnt polypeptide comprises a heterologous signal sequence or a native signal sequence. In some embodiments, the Wnt polypeptide comprises a tag, optionally a HIS(6×)-tag (SEQ ID NO: 19), a FLAG tag, or a PA tag. In some embodiments, the Wnt polypeptide is a Wnt3A polypeptide. In some embodiments, the Wnt3A polypeptide comprises about 90%, 95%, 99%, or more sequence identity to SEQ ID NO: 1. In some embodiments, the Wnt3A polypeptide comprises a truncation of about 1 to about 33 amino acids, optionally a C-terminal truncation. In some embodiments, the Wnt3A polypeptide comprises about 90%, 95%, 99%, or more sequence identity to SEQ ID NO: 2, or consists of SEQ ID NO: 2. In some embodiments, the Wnt polypeptide comprises a lipid modification at an amino acid position corresponding to amino acid residue 209 as set forth in SEQ ID NO: 1. In some embodiments, the Wnt polypeptide is modified with palmitic acid. In some embodiments, the second affinity chromatography column removes residual Frizzled-8 fusion proteins from the second Wnt composition. In some embodiments, the mixed mode column removes Wnt polypeptide fragments from the second Wnt composition. In some embodiments, the size exclusion chromatography column removes residual Wnt polypeptide fragments from the second Wnt composition to generate the third Wnt composition. In some embodiments, the second Wnt composition is greater than 60%, 65%, 70%, 75%, 80%, 85%, 90%, 95%, 96%, 97%, 98%, or 99% pure, relative to an equivalent Wnt composition that is purified in the absence of the sugar detergent. In some embodiments, the third Wnt composition is greater than 60%, 65%, 70%, 75%, 80%, 85%, 90%, 95%, 96%, 97%, 98%, or 99% pure, relative to an equivalent Wnt composition that is purified in the absence of the sugar detergent. In some embodiments, the final Wnt composition has a liposomal particle size distribution of: from about 10 nm to about 1 μm, from 10 nm to about 500 nm, from about 50 nm to about 300 nm, from about 50 nm to about 200 nm, from about 50 nm to about 150 nm, from about 100 nm to about 500 nm, from about 100 nm to about 300 nm, or from about 100 nm to about 200 nm; less than about 1 μm, less than about 500 nm, less than about 300 nm, less than about 200 nm, or less than about 150 nm; or about 50 nm, about 100 nm, or about 150 nm.

Disclosed herein, in certain embodiments, is a functionally active Wnt polypeptide generated by a method described above.

Disclosed herein, in certain embodiments, is a liposomal Wnt composition comprising a functionally active Wnt polypeptide generated by a method described above.

Disclosed herein, in certain embodiments, is a method of enhancing cell survival in a bone graft in a subject in need thereof, comprising: (a) incubating a sample comprising isolated mammalian bone graft material comprising cells ex-vivo with a composition comprising a liposomal Wnt polypeptide generated by a method described above; and (b) transplanting the enhanced cells into a target site. In some embodiments, the cells of step a) are incubated for at least 10 minutes, 20 minutes, 30 minutes, 1 hour, 2 hours, 3 hours, 6 hours, or more. In some embodiments, the cells of step a) are incubated at about room temperature or at about 37° C. In some embodiments, the enhanced cells comprise enhanced osteogenic capacity relative to unexposed mammalian bone graft material.

Disclosed herein, in certain embodiments, is a method of enhancing cell survival at a bone defect site in a subject in need thereof, comprising: administering to the bone defect site a composition comprising a liposomal Wnt polypeptide generated by a method described above, wherein the liposomal Wnt polypeptide enhances cell survival at the bone defect site. In some embodiments, the method further comprises administering a dental or orthopedic implant at the bone defect site. In some embodiments, the dental or orthopedic implant is administered to the bone defect site prior to administration of the composition comprising a liposomal Wnt polypeptide. In some embodiments, the dental or orthopedic implant is administered to the bone defect site after administration of the composition comprising a liposomal Wnt polypeptide. In some embodiments, the dental or orthopedic implant is administered to the bone defect site about 1 day, 2 days, 5 days, 7 days, 2 weeks, 30 days, 1 month, 2 months, 3 months, 4 months, 5 months, 6 months, or more after administration of the composition comprising a liposomal Wnt polypeptide. In some embodiments, the dental or orthopedic implant and the composition comprising a liposomal Wnt polypeptide are administered to the bone defect site simultaneously. In some embodiments, the liposomal Wnt polypeptide enhances osseointegration of the dental or orthopedic implant. In some embodiments, the subject is a human.

Disclosed herein, in certain embodiments, is a Wnt composition comprising a purified Wnt polypeptide intermediate and a sugar detergent at a concentration from about 0.1% to about 5% w/v. In some embodiments, the sugar detergent comprises a glucoside detergent. In some embodiments, the glucoside detergent is n-hexyl-β-D-glucopyranoside, n-heptyl-β-D-glucopyranoside, n-octyl-β-D-glucopyranoside, n-octyl-α-D-glucopyranoside, octyl β-D-1-thioglucopyranoside, n-octyl-β-D-galactopyranoside, n-nonyl-β-D-glucopyranoside, n-decyl-β-D-glucopyranoside, n-dodecyl-β-D-glucopyranoside, or methyl-6-O—(N-heptylcarbamoyl)-α-D-glucopyranoside. In some embodiments, the glucoside detergent is selected from n-octyl-β-D-glucopyranoside and octyl β-D-1-thioglucopyranoside. In some embodiments, the glucoside detergent is n-octyl-β-D-glucopyranoside. In some embodiments, the glucoside detergent is octyl β-D-1-thioglucopyranoside. In some embodiments, the sugar detergent comprises a maltoside detergent. In some embodiments, the maltoside detergent is n-decyl-β-D-maltopyranoside, n-dodecyl-β-D-maltopyranoside, or 6-cyclohexyl-1-hexyl-β-D-maltopyranoside. In some embodiments, the concentration of the sugar detergent is about 0.1%, 0.5%, 1%, 1.5%, or about 2% w/v. In some embodiments, the Wnt composition has a pH of about 5, 5.5, or 6. In some embodiments, the Wnt composition further comprises a buffer comprising acetate at a concentration of about 10 mM, 15 mM, 20 mM, 25 mM, 30 mM, 40 mM, or 50 mM. In some embodiments, the purified Wnt polypeptide intermediate is obtained from the steps of: (a) co-expressing a Wnt polypeptide and a chaperone in a cell in a conditioned media to generate a plurality of Wnt polypeptide-chaperone complexes; (b) harvesting the plurality of Wnt polypeptide-chaperone complexes from the conditioned media; (c) incubating the plurality of Wnt polypeptide-chaperone complexes with a buffer comprising a sugar detergent to generate a mixture comprising a first Wnt composition comprising a functionally inactive Wnt polypeptide and a chaperone composition; and (d) purifying the first Wnt composition from the mixture with a column immobilized with a sulfonated polyaromatic compound, an affinity chromatography column comprising a polypeptide that interacts with the Fc portion of an antibody, a mixed mode column, a size exclusion chromatography column, or a combination thereof, to generate the Wnt composition comprising the purified Wnt polypeptide intermediate and the sugar detergent. In some embodiments, the Wnt polypeptide is a Wnt3A polypeptide. In some embodiments, the Wnt3A polypeptide is polypeptide that comprises about 90%, 95%, 99%, or more sequence identity to SEQ ID NO: 1. In some embodiments, the Wnt3A polypeptide comprises a truncation of about 1 to about 33 amino acids, optionally a C-terminal truncation. In some embodiments, the Wnt3A polypeptide comprises about 90%, 95%, 99%, or more sequence identity to SEQ ID NO: 2, or consists of SEQ ID NO: 2. In some embodiments, the Wnt polypeptide comprises a lipid modification at an amino acid position corresponding to amino acid residue 209 as set forth in SEQ ID NO: 1. In some embodiments, the Wnt polypeptide is modified with palmitic acid. In some embodiments, the concentration of the purified Wnt polypeptide intermediate is from about 20 μg/mL to about 50 μg/mL, from about 25 μg/mL to about 50 μg/mL, from about 30 μg/mL to about 50 μg/mL, from about 20 μg/mL to about 40 μg/mL, from about 25 μg/mL to about 40 μg/mL, from about 25 μg/mL to about 30 μg/mL, from about 30 μg/mL to about 50 μg/mL, or from about 30 μg/mL to about 40 μg/mL; or about 20 μg/mL, about 25 μg/mL, about 30 μg/mL, about 35 μg/mL, about 40 μg/mL, about 45 μg/mL, or about 50 μg/mL.

Disclosed herein, in certain embodiments, is a Wnt culture system comprising: (a) minimal serum culture media; (b) a Wnt polypeptide-chaperone complex located in the minimal serum culture media; and (c) cells from an engineered cell line transfected with a first expression vector encoding the Wnt polypeptide and a second expression vector encoding the chaperone; wherein the Wnt polypeptide and the chaperone are co-expressed in the cells, and the cells are grown in the presence of the minimal serum culture media. In some embodiments, the chaperone comprises a Frizzled protein. In some embodiments, the chaperone comprises Wntless. In some embodiments, the chaperone comprises Afamin. In some embodiments, the chaperone comprises a Frizzled-8 fusion protein. In some embodiments, the Frizzled-8 fusion protein comprises a truncated Frizzled-8 protein. In some embodiments, the truncated Frizzled-8 protein comprises a cysteine-rich region (CRD) of Frizzled-8. In some embodiments, the truncated Frizzled-8 protein comprises the region spanning amino acid residue 1 to amino acid residue 151 or spanning amino acid residue 1 to amino acid residue 172 of SEQ ID NO: 4. In some embodiments, the Frizzled-8 fusion protein further comprises an IgG Fc portion. In some embodiments, the Frizzled-8 fusion protein comprises at least 80%, 85%, 90%, 95%, 96%, 97%, 98%, or 99% sequence identity to SEQ ID NO: 5. In some embodiments, the Frizzled-8 fusion protein comprises at least 80%, 85%, 90%, 95%, 96%, 97%, 98%, or 99% sequence identity to SEQ ID NO: 18. In some embodiments, the Wnt polypeptide comprises a tag. In some embodiments, the tag comprises a HIS-tag, a FLAG tag, or a PA tag. In some embodiments, the Wnt polypeptide comprises a heterologous signal sequence. In some embodiments, the Wnt polypeptide comprises a native signal sequence. In some embodiments, the Wnt polypeptide is a Wnt3A polypeptide, Wnt5B polypeptide, or Wnt10B polypeptide. In some embodiments, the Wnt polypeptide is a Wnt3A polypeptide. In some embodiments, the Wnt3A polypeptide comprises about 90%, 95%, 99%, or more sequence identity to SEQ ID NO: 1. In some embodiments, the Wnt3A polypeptide comprises a truncation of about 1 to about 33 amino acids. In some embodiments, the truncation is a C-terminal truncation. In some embodiments, the Wnt3A polypeptide is a polypeptide of SEQ ID NO: 1 with a C-terminal truncation. In some embodiments, the Wnt3A polypeptide comprises about 90%, 95%, 99%, or more sequence identity to SEQ ID NO: 2. In some embodiments, the Wnt3A polypeptide consists of SEQ ID NO: 2. In some embodiments, the engineered cell line is a cGMP-compatible cell line. In some embodiments, the cGMP-compatible cell line is a cGMP-compatible mammalian cell line. In some embodiments, the cGMP-compatible mammalian cell line is Chinese Hamster Ovary (CHO) cell line, human embryonic kidney (HEK) cell line, or baby hamster kidney (BHK) cell line. In some embodiments, the cGMP-compatible mammalian cell line is CHO-S or CHO-K1 derivative cell line. In some embodiments, the first expression vector and the second expression vector are each independently a cGMP-compatible vector. In some embodiments, the first expression vector and the second expression vector are each independently a mammalian vector. In some embodiments, the mammalian vector is OpticVec, pTarget, pcDNA4TO4, pcDNA4.0, UCOE expression vector, or GS System expression vector.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a comparison study of Wnt3A expression in the presence of exogenous Frizzled-8 fusion protein (Fz-151-Fc) or in the presence of co-expressed Frizzled-8 fusion protein (Fz-151-Fc).

FIG. 2A-FIG. 2B show co-expression of Frizzled-8 fusion protein (Fz-151-Fc) reduces Wnt3A aggregation (FIG. 2A) and further increases the amount of Wnt3A monomer (FIG. 2B). The Wnt3A polypeptide was produced from a stable cell line.

FIG. 3 illustrates four exemplary purification strategies described herein.

FIG. 4A-FIG. 4D illustrate purification details of strategy 1. FIG. 4A shows an exemplary purification scheme for Strategy 1. FIG. 4B shows the silver staining of the various fractions. The condition is a non-reducing condition. FIG. 4C shows a western blot analysis of the various fractions to determine the presence and concentration of Wnt3A polypeptide. FIG. 4D illustrates the activity of the Wnt3A polypeptide in a LSL assay.

FIG. 5A-FIG. 5D illustrate purification details for strategy 2. FIG. 5A illustrates a Coomassie staining of Protein A fractions. FIG. 5B shows the silver staining of the various fractions. FIG. 5C shows a western blot analysis of the various fractions to determine the presence and concentration of Wnt3A polypeptide. FIG. 5D illustrates the activity of the Wnt3A polypeptide in a LSL assay.

FIG. 6A-FIG. 6B illustrate purification details for strategy 3. FIG. 6A shows the silver staining of the various fractions. FIG. 6B illustrates the activity of the Wnt3A polypeptide in a LSL assay.

FIG. 7A-FIG. 7C illustrate purification details for strategy 4. FIG. 7A shows a Coomassie staining of Protein A fractions. FIG. 7B shows the silver staining of the various fractions. FIG. 7C illustrates the activity of the Wnt3A polypeptide in a LSL assay.

FIG. 8A-FIG. 8C illustrate co-expression of a Wnt3A polypeptide with Wntless (WLS). FIG. 8A shows an increase in Wnt3A expression in the presence of co-expressed Wntless. FIG. 8B shows the activity of Wnt3A polypeptide in a LSL assay. FIG. 8C shows expression of Wnt3A in a stable cell line.

FIG. 9 illustrates co-expression of Wnt3A with Afamin.

FIG. 10A-FIG. 10B illustrate the expression and activity of three exemplary Wnt3A polypeptides tagged with: PA, FLAG, and His-tag, respectively. FIG. 10A illustrates the concentration of the secreted tagged Wnt3A polypeptides. FIG. 10B shows the activity of Wnt3A polypeptides in a LSL assay.

FIG. 11 shows the activity of Wnt3A variants (ART352^{his} variants) comprising different His-tag-linker constructs.

FIG. 12 shows the activity of the various fractions of the Wnt3A variant-ART352^{his} from a Ni-NTA column.

FIG. 13A-FIG. 13C show the concentration of the Wnt3A polypeptides in an ELISA assay.

FIG. 14 illustrates a purification scheme for purification of a FLAG-tagged Wnt3A polypeptide: FLAG-TEV-hWnt3A.

FIG. 15A-FIG. 15F show the activity and concentration of the FLAG-tagged Wnt3A polypeptide. FIG. 15A-FIG. 15C show the activity of the Wnt3A polypeptide in a LSL assay. FIG. 15D-FIG. 15F show the concentration of the Wnt3A polypeptide.

FIG. 16A-FIG. 16C show the activity of the Wnt3A cultured from a 0.75 L culture. FIG. 16A: fractions obtained from a heparin purification; FIG. 16B: illustrates the standard deviation; FIG. 16C: LUC/LAC per heparin fraction.

FIG. 17A-FIG. 17F show the activity and concentration of Wnt3A cultured from the 10 L culture. FIG. 17A: fractions obtained from a heparin purification; FIG. 17B: illustrates the standard deviation; FIG. 17C: LUC/LAC per heparin fraction; FIG. 17D: concentration of Wnt3A per fraction collected; FIG. 17E: illustrates the standard deviation in reference to FIG. 17D; and FIG. 17F: illustrates the final concentration from exemplary fractions.

FIG. 18 illustrates the activity of Wnt3A in complex with hFZD8 CRD-Fc.

FIG. 19 illustrates an exemplary purification scheme described herein.

FIG. 20A-FIG. 20B show exemplary gel images of Wnt3A (ART352) purification with either 1% CHAPS (FIG. 20A) or 1% OGP (FIG. 20B).

FIG. 21A-FIG. 21B illustrate LSL activity of WNT3A (ART352) eluates in 1% OGP (FIG. 21A) or 1% CHAPS (FIG. 21B).

FIG. 22 illustrates an exemplary gel image of purification with a mixed mode column.

FIG. 23A-FIG. 23B illustrate Wnt3A polypeptide purified with either buffer comprising 1% CHAPS (FIG. 23A) or 1% OGP (FIG. 23B).

FIG. 24A-FIG. 24B illustrate that OGP stabilizes WNT3A protein at 2 different temperatures, 4° C. (FIG. 24A) and 23° C. (FIG. 24B) in comparison to CHAPS.

FIG. 25 illustrates an exemplary liposomal Wnt3A formulation process.

FIG. 26 illustrates a representative standard curve using the exemplary Wnt3A polypeptide ART352. The sensitivity range was from about 0.003 μg/mL to about 1.6 μg/mL.

FIG. 27 shows the effect of solution conditions on cell viability in an autograft. Compared to the zero-time point (white bar), incubation in saline for 2 h leads to a doubling in the percentage of apoptotic cells in an autograft. In contrast, incubation in ART352-L reduces the time- and temperature-dependent increase in apoptosis, back to levels observed in control autografts.

FIG. 28 shows the temperature effect on cell viability in an autograft. For samples held in saline, a hold temperature of 4° C. reduces cell death in the autograft, while a hold temperature of 37° C. increases cell death in the autograft.

FIG. 29 shows the effect of time and temperature on endocytosis of exemplary liposomal Wnt3A polypeptide ART352-L. Endocytosis of DiI labeled ART352-L increases as a function of time and temperature. These data suggest that for the intended duration of the ex vivo hold, from 15 min to 2 h, incubation at 37° C. supports nutrient uptake. Data in demonstrate that the uptake of ART352-L ameliorates cell death associated with standard autograft handling.

FIG. 30 shows ART352-L stability as a function of time and temperature. Over the course of 2 h, ART352-L shows a minimal (4.9%) loss in activity.

FIG. 31 shows the endocytic removal of active ART352-L from the incubation solution. In the absence of an autograft, ART352-L levels remain at 100% in the incubation solution. In the presence of an autograft, the removal of active ART352-L from the solution occurs in a temperature- and time-dependent manner.

FIG. 32 shows an assessment of free, active ART352-L associated with a ART352-L treated autograft. ART352-L treated autografts show no evidence of residual, free, active ART352 regardless of the temperature or duration of the ex vivo incubation step.

FIG. 33 shows ART352-L removal from incubation solution and uptake by the cells derived from the autografts.

### DETAILED DESCRIPTION OF THE DISCLOSURE

Wnts are involved in a wide variety of cellular decisions associated with the program of osteogenesis. For example, Wnts regulate the expression level of sox9 and Runx2, two transcription factors that influence the commitment of mesenchymal progenitor cells to a skeletogenic fate. Wnts also influence the differentiation of cells, into either osteoblasts or chondrocytes. In adult animals, there is abundant evidence that Wnt signaling regulates bone mass. For example, gain of function mutations that increase Wnt signaling are associated with several high bone mass syndromes, including osteoporosis type I, and endosteal hyperostosis or autosomal dominant osteosclerosis. Loss of function mutations that reduce Wnt signaling cause a low bone mass disease, osteoporosis-pseudoglioma. Increased production of the Wnt inhibitor Dkk1 is associated with multiple myeloma, a disease that has increased bone resorption as one of its distinguishing features, and loss of the Wnt inhibitor Sclerostin is associated with high bone mass diseases including sclerostosis and van Buchem disease.

The role of Wnt signaling in cellular decisions has been determined in large part by experiments conducted in vitro in which Wnt signaling is abrogated or blocked. In some instances, Wnt signal is blocked by an excess of the ligand binding domain of its receptor, Frizzled.

Wnt polypeptides comprise a family of signaling molecules that orchestrates cellular developmental and biological processes. In some instances, Wnt polypeptides modulate stem cell self-renewal, apoptosis, and cell motility. In other instances, Wnt polypeptides contribute to development, such as for example, tissue homeostasis. The Wnt polypeptide is a highly hydrophobic protein and under some instances (e.g., certain media conditions) has reduced or loses biological function. In some cases, formulation of a Wnt polypeptide with an exogenous agent (e.g., a liposome) allows the Wnt polypeptide to maintain biological function. For example, it has been shown that combining a Wnt polypeptide with a lipid vesicle (e.g., a liposome) produce a Wnt formulation (Morrell N T, Leucht P, Zhao L, Kim J-B, ten Berge D, et al. (2008) Liposomal Packaging Generates Wnt Protein with In Vivo Biological Activity. PLoS ONE 3(8): e2930; and Zhao et al., Controlling the in vivo activity of Wnt liposomes, Methods Enzyrnol 465: 331-47 (2009)) with biological activity (Minear et al., Wnt proteins promote bone regeneration. Sci. Transl. Med. 2, 29ra30 (2010); and Popelut et al., The acceleration of implant osseointegration by liposomal Wnt3A, Biomaterials 31 9173e9181 (2010); U.S. Pat. Nos. 7,335,643 and 7,153,832).

In some instances, Wnt polypeptides are secreted from culture cells in the presence of serum. Serum contains a variety of lipid components, which in some cases stabilize the highly hydrophobic Wnt polypeptide in vitro. The hydrophobicity is based on the presence of palmitoylation, which are required for Wnt activity. For safety reasons, however, regulatory bodies including the FDA and EMA generally require the removal of all animal products from drugs intended for use in humans. Additionally, fetal bovine serum used in the manufacture of FDA-regulated medical products is prohibited if appropriate procedures have not been followed to prevent contamination with viruses and other pathogens.

In some cases, Wnt polypeptides are stabilized by surfactants. Although surfactants protect the hydrophobic Wnt from aggregation; however, the concentration level that is capable of stabilizing a Wnt polypeptide is cytotoxic to human cells, in some cases leading to cell lysis.

Disclosed herein are methods and culture systems of producing Wnt polypeptides under minimal serum condition (e.g., serum-free condition). In some embodiments, a method described herein comprises (a) incubating a plurality of Wnt polypeptide-chaperone complexes with a buffer comprising a sugar detergent to generate a mixture comprising a first Wnt composition comprising a functionally inactive Wnt polypeptide and a chaperone composition; (b) separating the first Wnt composition from the mixture with a column immobilized with a sulfonated polyaromatic compound to generate a second Wnt composition comprising the functionally active Wnt polypeptide and the sugar detergent; (c) optionally purifying the second Wnt composition with an affinity chromatography column comprising a polypeptide that interacts with the Fc portion of an antibody, a mixed mode column, a size exclusion chromatography column, or a combination thereof, at least once to generate a third Wnt composition; and (d) contacting the second Wnt composition or optionally the third Wnt composition with an aqueous solution of liposomes to generate a final Wnt composition comprising a functionally active Wnt polypeptide.

In some embodiments, a method described herein comprises (a) coexpressing a Wnt polypeptide with a chaperone in a cell in a conditioned media to generate a Wnt polypeptide-chaperone complex; (b) harvesting the Wnt polypeptide-chaperone complex from the conditioned media; (c) introducing the Wnt polypeptide-chaperone complex to a column immobilized with a sulfonated polyaromatic compound to generate an eluted Wnt polypeptide-chaperone complex; (d) processing the eluted Wnt polypeptide-chaperone complex through an affinity chromatography column comprising a polypeptide that interacts with the Fc portion of an antibody to generate a processed Wnt polypeptide; and (e) contacting the processed Wnt polypeptide with an aqueous solution of liposomes to generate the liposomal Wnt polypeptide

In some embodiments, a method described herein comprises (a) coexpressing a Wnt polypeptide with a chaperone in a cell in a conditioned media to generate a Wnt polypeptide-chaperone complex; (b) harvesting the Wnt polypeptide-chaperone complex from the conditioned media; (c) introducing the Wnt polypeptide-chaperone complex to an affinity chromatography column comprising a polypeptide that interacts with the Fc portion of an antibody to generate an eluted Wnt polypeptide-chaperone complex; (d) processing the eluted Wnt polypeptide-chaperone complex through a column immobilized with a sulfonated polyaromatic compound to generate a processed Wnt polypeptide; and (e) contacting the processed Wnt polypeptide with an aqueous solution of liposomes to generate the liposomal Wnt polypeptide.

In additional embodiments, also described herein include a Wnt culture system, which comprises (a) minimal serum culture media; (b) a Wnt polypeptide-chaperone complex located in the minimal serum culture media; and (c) cells from an engineered cell line transfected with a first expression vector encoding the Wnt polypeptide and a second expression vector encoding the chaperone; wherein the Wnt polypeptide and the chaperone are co-expressed in the cells, and the cells are grown in the presence of the minimal serum culture media.

### Wnt Polypeptide

Wnt polypeptides or proteins form a family of highly conserved secreted signaling molecules that regulate cell-to-cell interactions during embryogenesis. In some embodiments, Wnt polypeptides include Wnt1, Wnt2, Wnt2B (or Wnt13), Wnt3, Wnt3A, Wnt4, Wnt5A, Wnt5B, Wnt6, Wnt7A, Wnt7B, Wnt8A, Wnt8B, Wnt9A (Wnt14, or Wnt14B), Wnt9B (Wnt14B, or Wnt15), Wnt10A, Wnt10B (or Wnt12), Wnt11, Wnt-16A, and Wnt-16B polypeptide. In some embodiments, a Wnt polypeptide is selected from Wnt3A polypeptide, Wnt5A polypeptide, and Wnt10B polypeptide. In some embodiments, the Wnt polypeptide is Wnt3A polypeptide. In some embodiments, the Wnt polypeptide is Wnt5A polypeptide. In some embodiments, the Wnt polypeptide is Wnt10B polypeptide. The terms “Wets” or “Wnt gene product” or “Wnt polypeptide” when used herein encompass native sequence Wnt polypeptides, Wnt polypeptide variants, Wnt polypeptide fragments and chimeric Wnt polypeptides.

A “native sequence” polypeptide is one that has the same amino acid sequence as a Wnt polypeptide derived from nature. Such native sequence polypeptides can be isolated from cells producing endogenous Wnt protein or can be produced by recombinant or synthetic means. Thus, a native sequence polypeptide can have the amino acid sequence of, e.g. naturally occurring human polypeptide, murine polypeptide, or polypeptide from any other mammalian species, or from non-mammalian species, e.g. Drosophila, C. elegans, and the like.

The term “native sequence Wnt polypeptide” includes, without limitation, Wnt1, Wnt2, Wnt2B (or Wnt13), Wnt3, Wnt3A, Wnt4, Wnt5A, Wnt5B, Wnt6, Wnt7A, Wnt7B, Wnt8A, Wnt8B, Wnt9A (Wnt14, or Wnt14B), Wnt9B (Wnt14B, or Wnt15), Wnt10A, Wnt10B (or Wnt12), Wnt11, Wnt-16A, and Wnt-16B polypeptide. In some instances, the term “native sequence Wnt polypeptide” includes human Wnt polypeptides. In some cases, the human Wnt polypeptides include human Wnt1, Wnt2, Wnt2B (or Wnt13), Wnt3, Wnt3A, Wnt4, Wnt5A, Wnt5B, Wnt6, Wnt7A, Wnt7B, Wnt8A, Wnt8B, Wnt9A (Wnt14, or Wnt14B), Wnt9B (Wnt14B, or Wnt15), Wnt10A, Wnt10B (or Wnt12), Wnt11, Wnt-16A, and Wnt-16B polypeptide. In some cases, the human Wnt polypeptide is human Wnt3A polypeptides. In some cases, the human Wnt polypeptide is human Wnt5A. In additional cases, the human Wnt polypeptide is human Wnt10B.

In some instances, Wnt1 is referred by the GenBank references NP005421.1 and AAH74799.1. Wnt2 is referred by the GenBank references NP003382.1 and AAH78170.1 In general, Wnt2 is expressed in the brain, thalamus, in both fetal and adult lungs, or in the placenta. Wnt2B has two isoforms and their GenBank reference Nos. are NP004176.2 and NP078613.1, respectively. In some cases, isoform 1 is expressed in adult heart, brain, placenta, lung, prostate, testis, ovary, small intestine and/or colon. In the adult brain, it is mainly found in the caudate nucleus, subthalamic nucleus and thalamus. In some instances, it is also detected in fetal brain, lung and kidney. In some cases, isoform 2 is expressed in fetal brain, fetal lung, fetal kidney, caudate nucleus, testis, and/or cancer cell lines.

Wnt3 and Wnt3A play distinct roles in cell-cell signaling during morphogenesis of the developing neural tube. In some instances, the mRNA sequence for human Wnt3 has the GenBank reference AB067628.1, and the protein sequence for human Wnt3 has the GenBank reference BAB70502.1. The mRNA sequence for human Wnt3A has the GenBank reference AB060284.1 and the protein sequence for human Wnt3A has the GenBank Nos. BAB61052.1 and AAI03924.1. Additionally, human Wnt3A has the GenBank accession number BC103922 and the accession number BC103921. In some instances, the term “native sequence Wnt protein” or “native sequence Wnt polypeptide” includes the Wnt3A native polypeptides (e.g., polypeptides of accession numbers BAB61052.1 and AAI03924.1) with or without the initiating N-terminal methionine (Met), and with or without the native signal sequence. In some cases, the terms include the 352 amino acids native human Wnt3A polypeptide of SEQ ID NO: 2, without or without its N-terminal methionine (Met), and with or without the native signal sequence.

In some embodiments, Wnt4 has the GenBank references NP1 10388.2 and BAC23080.1. Wnt5A has the GenBank references NP003383.1, and NP003383.2. Wnt5B has the GenBank references BAB62039.1 and AAG38659. Wnt6 has the GenBank references NP006513.1 and BAB55603.1. Wnt7A has the GenBank references NP004616.2 and BAA82509.1. In some instances, it is expressed in the placenta, kidney, testis, uterus, fetal lung, fetal brain, or adult brain. Wnt7B has the GenBank references NP478679.1 and BAB68399.1. In some cases, it is expressed in fetal brain, lung and/or kidney, or in adult brain, lung and/or prostate. Wnt8A has at least two alternative transcripts, GenBank references NP114139.1 and NP490645.1. Wnt8B is expressed in the forebrain. It has the GenBank reference NP003384.1. Wnt1 OA has the GenBank references AAG45153 and NP079492.2. Wnt10B is detected in most adult tissues, with highest levels in the heart and skeletal muscles. It has the GenBank reference NP003385.2. In some cases, Wnt11 is expressed in fetal lung, kidney, adult heart, liver, skeletal muscle, and pancreas. It has the Genbank reference NP004617.2. Wnt14 has the Genbank reference NP003386.1. Wnt15 is expressed in fetal kidney or adult kidney, or expressed in the brain. It has the GenBank reference NP003387.1. Wnt16 has two isoforms, Wnt-16A and Wnt-16B, produced by alternative splicing. Isoform Wnt-16A is expressed in the pancreas. Isoform Wnt-16B is expressed in peripheral lymphoid organs such as spleen, appendix, and lymph nodes, or in the kidney, but not expressed in bone marrow. The GenBank references are NP476509.1 and NP057171.2, respectively, for Wnt16A and Wnt16B. All GenBank, SwissProt and other database sequences listed are expressly incorporated by reference herein.

A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid.

In some instances, a biologically active Wnt variant has an amino acid sequence having at least about 80% amino acid sequence identity with a native sequence Wnt polypeptide. In some instances, the biologically active Wnt variant has an amino acid sequence having at least about 85%, 90%, 91%, 92%, 93%, 94%, 95%, 96%, 96%, 97%, or 99% amino acid sequence identity with a native sequence Wnt polypeptide. In some cases, the biologically active Wnt variant has an amino acid sequence having at least about 95% amino acid sequence identity with a native sequence Wnt polypeptide. In some cases, the biologically active Wnt variant has an amino acid sequence having at least about 99% amino acid sequence identity with a native sequence Wnt polypeptide. In some embodiments, the biologically active Wnt variant is Wnt3A, Wnt5A, or Wnt10B. In some embodiments, the biologically active Wnt variant is a Wnt3A variant, e.g., the amino acid sequence of the Wnt3A variant having at least about 85%, 90%, 91%, 92%, 93%, 94%, 95%, 96%, 96%, 97%, or 99% amino acid sequence identity with the native sequence of Wnt3A. In some embodiments, the biologically active Wnt variant is a human Wnt3A variant.

In some embodiments, a biologically active Wnt variant is a truncated Wnt polypeptide. In some instances, the truncation is from the N-terminus. In other instances the truncation is from the C-terminus. In some cases, the Wnt polypeptide is truncated by between 5 to 40 amino acids, by between 5 to 35 amino acids, between 10 to 35 amino acids, between 10 to 33 amino acids, between 10 to 30 amino acids, between 15 to 33 amino acids, between 15 to 30 amino acids, between 20 to 35 amino acids, between 20 to 33 amino acids, between 20 to 30 amino acids, between 25 to 33 amino acids, or between 25 to 30 amino acids. In some cases, the Wnt polypeptide is truncated at the C-terminus by between 5 to 40 amino acids, by between 5 to 35 amino acids, between 10 to 35 amino acids, between 10 to 33 amino acids, between 10 to 30 amino acids, between 15 to 33 amino acids, between 15 to 30 amino acids, between 20 to 35 amino acids, between 20 to 33 amino acids, between 20 to 30 amino acids, between 25 to 33 amino acids, or between 25 to 30 amino acids. In some cases, the truncated Wnt polypeptide is a truncated Wnt3A polypeptide, a truncated Wnt5A polypeptide, or a truncated Wnt10B polypeptide.

In some embodiments, the Wnt polypeptide is truncated at the C-terminus by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more amino acids. In some cases, the Wnt polypeptide is truncated at the C-terminus by 5 or more amino acids. In some cases, the Wnt polypeptide is truncated at the C-terminus by 10 or more amino acids. In some cases, the Wnt polypeptide is truncated at the C-terminus by 15 or more amino acids. In some cases, the Wnt polypeptide is truncated at the C-terminus by 20 or more amino acids. In some cases, the Wnt polypeptide is truncated at the C-terminus by 25 or more amino acids. In some cases, the Wnt polypeptide is truncated at the C-terminus by 30 or more amino acids. In some cases, the Wnt polypeptide is truncated at the C-terminus by 31 or more amino acids. In some cases, the Wnt polypeptide is truncated at the C-terminus by 32 or more amino acids. In some cases, the Wnt polypeptide is truncated at the C-terminus by 33 or more amino acids. In some cases, the Wnt polypeptide is truncated at the C-terminus by 34 or more amino acids. In some cases, the Wnt polypeptide is truncated at the C-terminus by 35 or more amino acids. In some cases, the Wnt polypeptide is additionally truncated at the N terminus, provided that the polypeptide maintains biological activity. In some cases, the truncated Wnt polypeptide is a truncated Wnt3A polypeptide, a truncated Wnt5A polypeptide, or a truncated Wnt10B polypeptide.

In some embodiments, the truncated Wnt polypeptide is a truncated Wnt3A polypeptide. In some instances, the truncation is from the N-terminus. In other instances the truncation is from the C-terminus. In some cases, the Wnt3A polypeptide is truncated by between 5 to 40 amino acids, by between 5 to 35 amino acids, between 10 to 35 amino acids, between 10 to 33 amino acids, between 10 to 30 amino acids, between 15 to 33 amino acids, between 15 to 30 amino acids, between 20 to 35 amino acids, between 20 to 33 amino acids, between 20 to 30 amino acids, between 25 to 33 amino acids, or between 25 to 30 amino acids. In some cases, the Wnt3A polypeptide is truncated at the C-terminus by between 5 to 40 amino acids, by between 5 to 35 amino acids, between 10 to 35 amino acids, between 10 to 33 amino acids, between 10 to 30 amino acids, between 15 to 33 amino acids, between 15 to 30 amino acids, between 20 to 35 amino acids, between 20 to 33 amino acids, between 20 to 30 amino acids, between 25 to 33 amino acids, or between 25 to 30 amino acids.

In some embodiments, the Wnt3A polypeptide is truncated at the C-terminus by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more amino acids. In some cases, the Wnt3A polypeptide is truncated at the C-terminus by 5 or more amino acids. In some cases, the Wnt3A polypeptide is truncated at the C-terminus by 10 or more amino acids. In some cases, the Wnt3A polypeptide is truncated at the C-terminus by 15 or more amino acids. In some cases, the Wnt3A polypeptide is truncated at the C-terminus by 20 or more amino acids. In some cases, the Wnt3A polypeptide is truncated at the C-terminus by 25 or more amino acids. In some cases, the Wnt3A polypeptide is truncated at the C-terminus by 30 or more amino acids. In some cases, the Wnt3A polypeptide is truncated at the C-terminus by 31 or more amino acids. In some cases, the Wnt3A polypeptide is truncated at the C-terminus by 32 or more amino acids. In some cases, the Wnt3A polypeptide is truncated at the C-terminus by 33 or more amino acids. In some cases, the Wnt3A polypeptide is truncated at the C-terminus by 34 or more amino acids. In some cases, the Wnt3A polypeptide is truncated at the C-terminus by 35 or more amino acids. In some cases, the Wnt3A polypeptide is additionally truncated at the N terminus, provided that the polypeptide maintains biological activity.

In some instances, a biologically active Wnt variant comprises a lipid modification at one or more amino acid positions. In some cases, the lipid modification is at a position on a Wnt variant that is equivalent to position 209 set forth in SEQ ID NO: 1. In some instances, the Wnt variant is Wnt3A, Wnt5A, or Wnt 10B. In some cases, the Wnt variant is Wnt3A. In some cases, the Wnt3A variant comprises a lipid modification at a position equivalent to residue 209 set forth in SEQ ID NO: 1. In some cases, the Wnt polypeptide is modified with a fatty acid, e.g., a saturated fatty acid or an unsaturated fatty acid. In some cases, the Wnt polypeptide is modified with an unsaturated fatty acid (e.g., a mono-unsaturated fatty acid such as palmitoleic acid). In other cases, the Wnt polypeptide is modified with a saturated fatty acid (e.g., palmitic acid). In additional cases, the Wnt polypeptide is modified with palmitic acid. In some instances, the modification is palmitoylation. In some instances, the Wnt3A variant is a truncated Wnt3A polypeptide, which comprises a lipid modification (e.g., a saturated fatty acid modification such as palmitic acid) at a position equivalent to residue 209 set forth in SEQ ID NO: 1.

In some instances, a biologically active Wnt variant further comprises a residue modified by glycosylation. In some cases, the modification occurs at a position equivalent to position 82 and/or 298 set forth in SEQ ID NO: 1. In some instances, the Wnt variant is Wnt3A, Wnt5A, or Wnt 10B. In some cases, the Wnt variant is Wnt3A. In some cases, a Wnt3A variant further comprises a residue modified by glycosylation. In some cases, a Wnt3A variant further comprises a glycosylated residue at one or more positions equivalent to residue 82 and/or residue 298 set forth in SEQ ID NO: 1. In some cases, the Wnt3A variant is a truncated Wnt3A polypeptide.

In some embodiments, a biologically active Wnt variant further comprises a tag. In some instances, the tag is an affinity tag. In other instances, the tag is an epitope tag. Exemplary tags described herein include, but are not limited to, poly-histidine tag, PA-tag, FLAG tag, human influenza hemagglutinin (HA) tag, Myc tag, glutathione-S transferase (GST), calmodulin binding protein (CBP), maltose-binding protein (MBP), ABDz1-tag (albumin), HaloTag®, heparin-binding peptide (HB) tag, poly-Arg tag, poly-Lys tag, S-tag, Strep-II tag, and SUMO tag. In some instances, the poly-histidine tag comprises about 6 to 12, about 6 to 10, or about 6 to 8 histidine residues in tandem. In some instances, the poly-histidine tag comprises about 6 to 10 histidine residues in tandem. In some instances, the poly-histidine tag comprises about 6 to 8 histidine residues in tandem. In some cases, the poly-histidine tag comprises about 10 histidine residues (10×His (SEQ ID NO: 20)). In some cases, the poly-histidine tag comprises about 6 histidine residues (6×His (SEQ ID NO: 19)). In some instances, the PA-tag comprises a dodecapeptide from the anti-human podoplanin antibody NZ-1. In some cases, the dodecapeptide comprises the sequence GVAMPGAEDDVV (SEQ ID NO: 21). In some instances, the FLAG tag is a small peptide tag and optionally comprises the sequence DYKDDDDK (SEQ ID NO: 22). In some instances, the HA-tag is derived from the surface glycoprotein that facilitates the ability of the influenza virus to infect its host and optionally comprises the sequence YPYDVPDYA (SEQ ID NO: 23). In some instances, the Myc tag is derived from the Myc protein encoded by the c-Myc gene and optionally comprises the sequence EQKLISEEDL (SEQ ID NO: 24). In some instances, the Wnt variant is Wnt3A, Wnt5A, or Wnt 10B. In some cases, the Wnt variant is Wnt3A. In some cases, the Wnt3A variant is a truncated Wnt3A polypeptide.

In some embodiments, the tag is directly connected to the biologically active Wnt variant. In such cases, the tag is directly connected to the N-terminus of the biologically active Wnt variant. In other cases, the tag is directly connected to the C-terminus of the biologically active Wnt variant. In some instances, the Wnt variant is Wnt3A, Wnt5A, or Wnt 10B. In some cases, the Wnt variant is Wnt3A. In some cases, the Wnt3A variant is a truncated Wnt3A polypeptide.

In some embodiments, the tag is indirectly connected to the biologically active Wnt variant through a linker. In some cases, the linker is a cleavable linker, comprising, e.g., a thrombin, Factor Xa, TEV, or an enterokinase polypeptide motif. In some cases, the thrombin cleavable linker comprises a LVPRGS (SEQ ID NO: 25) recognition motif. In some cases, the Factor Xa linker comprises a consensus site I-(E/D)-G-R. In some cases, the TEV linker comprises a consensus site E-N-L-Y-F-Q-(G/S) (SEQ ID NO: 26). In some cases, the enterokinase linker comprises the motif DDDDK (SEQ ID NO: 27). In some cases, the tag is indirectly connected, through a linker, to the C-terminus of the biologically active Wnt variant. In other cases, the tag is indirectly connected, through a linker, to the N-terminus of the biologically active Wnt variant. In some instances, the Wnt variant is Wnt3A, Wnt5A, or Wnt 10B. In some cases, the Wnt variant is Wnt3A. In some cases, the Wnt3A variant is a truncated Wnt3A polypeptide.

In some instances, the linker is a non-cleavable linker. In some cases, the non-cleavable linker comprises, e.g., a poly-glycine residues, a poly-alanine residues, or a combination of glycine and alanine residues. Exemplary non-cleavable linkers include, but are not limited to, GGG, GGGGGG (SEQ ID NO: 28), and GGGGAGGGG (SEQ ID NO: 29). In some instances, the Wnt variant is Wnt3A, Wnt5A, or Wnt 10B. In some cases, the Wnt variant is Wnt3A. In some cases, the Wnt3A variant is a truncated Wnt3A polypeptide.

In some instances, the biologically active Wnt variant comprises one or more tags (e.g., 2, 3, 4, 5, or more tags). In some cases, one or more tags are connected either directly or indirectly through a linker to the N-terminus of the biologically active Wnt variant, and optionally one or more additional tags are connected either directly or indirectly through a linker to the C-terminus of the biologically active Wnt variant. In one embodiment, the N-terminus of the biologically active Wnt variant comprises a poly-histidine tag (e.g., indirectly via a linker) and the C-terminus of the biologically active Wnt variant comprises an additional tag. In another embodiment, the C-terminus of the biologically active Wnt variant comprises a poly-histidine tag (e.g., indirectly via a linker) and the N-terminus of the biologically active Wnt variant comprises an additional tag. In some instances, the Wnt variant is Wnt3A, Wnt5A, or Wnt 10B. In some cases, the Wnt variant is Wnt3A. In some cases, the Wnt3A variant is a truncated Wnt3A polypeptide.

The term “amino acid” refers to a molecule containing both an amino group and a carboxyl group. Suitable amino acids include, without limitation, both the D- and L-isomers of the naturally-occurring amino acids, as well as non-naturally occurring amino acids prepared by organic synthesis or other metabolic routes. The term amino acid, as used herein, includes, without limitation, α-amino acids, natural amino acids, non-natural amino acids, and amino acid analogs.

The term “α-amino acid” refers to a molecule containing both an amino group and a carboxyl group bound to a carbon which is designated the α-carbon.

The term “β-amino acid” refers to a molecule containing both an amino group and a carboxyl group in a 13 configuration.

The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The following Table 1 shows a summary of the properties of natural amino acids:

| | 3- | 1- | Side- | Side-chain | |
|---|---|---|---|---|---|
| | Letter | Letter | chain | charge | Hydropathy |
| Amino Acid | Code | Code | Polarity | (pH 7.4) | index |
| Alanine | Ala | A | nonpolar | neutral | 1.8 |
| Arginine | Avg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive (10%) | −3.2 |
| | | | | neutral (90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acid” are glycine, alanine, proline, and analogs thereof “Large hydrophobic amino acids” are valine, leucine, isoleucine, phenylalanine, methionine, tryptophan, and analogs thereof “Polar amino acids” are serine, threonine, asparagine, glutamine, cysteine, tyrosine, and analogs thereof “Charged amino acids” are lysine, arginine, histidine, aspartate, glutamate, and analogs thereof.

The term “amino acid analog” refers to a molecule which is structurally similar to an amino acid and which can be substituted for an amino acid in the formation of a peptidomimetic macrocycle Amino acid analogs include, without limitation, β-amino acids and amino acids where the amino or carboxy group is substituted by a similarly reactive group (e.g., substitution of the primary amine with a secondary or tertiary amine, or substitution of the carboxy group with an ester).

The term “non-natural amino acid” refers to an amino acid which is not one of the twenty amino acids commonly found in peptides synthesized in nature, and known by the one letter abbreviations A, R, N, C, D, Q, E, G, H, I, L, K, M, F, P, S, T, W, Y and V. Non-natural amino acids or amino acid analogs include, without limitation, the following amino acid analogs.

Amino acid analogs include β-amino acid analogs. Examples of β-amino acid analogs include, but are not limited to, the following: cyclic β-amino acid analogs; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl) butyric acid; (S)-3-amino-4-(4-cyanophenyl)-butyric acid; (S)-3-amino-4-(4-fluorophenyl) butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-3-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogs include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanin; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; 3-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine-dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexylammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogs include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; Nδ-benzyloxycarbonyl-L-ornithine; Nω-nitro-D-arginine; Nω-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-ornithine; D-ornithine; L-ornithine; Arg(Me)(Pbf)-OH; Arg(Me)₂-OH (asymmetrical); Arg(Me)2-OH (symmetrical); Lys(ivDde)-OH; Lys(Me)2-OH.HCl; Lys(Me3)-OH chloride; Nω-nitro-D-arginine; and Nω-nitro-L-arginine.

Amino acid analogs include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

Amino acid analogs include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogs include analogs of phenylalanine and tyrosine. Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogs include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Amino acid analogs include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogs include analogs of tryptophan. Examples of amino acid analogs of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, amino acid analogs are racemic. In some embodiments, the D isomer of the amino acid analog is used. In some embodiments, the L isomer of the amino acid analog is used. In other embodiments, the amino acid analog comprises chiral centers that are in the R or S configuration. In still other embodiments, the amino group(s) of a β-amino acid analog is substituted with a protecting group, e.g., tert-butyloxycarbonyl (BOC group), 9-fluorenylmethyloxycarbonyl (FMOC), tosyl, and the like. In yet other embodiments, the carboxylic acid functional group of a β-amino acid analog is protected, e.g., as its ester derivative. In some embodiments the salt of the amino acid analog is used.

A “non-essential” amino acid residue is a residue that can be altered from the wild-type sequence of a polypeptide without abolishing or substantially altering its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, for example, is replaced with another amino acid residue from the same side chain family. Other examples of acceptable substitutions are substitutions based on isosteric considerations (e.g. norleucine for methionine) or other properties (e.g. 2-thienylalanine for phenylalanine, or 6-Cl-tryptophan for tryptophan).

### Culturing Wnt Polypeptide Under Serum-Free Conditions

Disclosed herein, in some embodiments, are methods of producing a Wnt polypeptide under serum-free conditions. In some embodiments, the Wnt polypeptide is co-expressed with a chaperone. In some cases, the Wnt polypeptide forms a complex with the co-expressed chaperone, and the Wnt polypeptide-chaperone complex stabilizes Wnt polypeptide and enhances Wnt polypeptide expression. In some instances, the Wnt polypeptide is a biologically active Wnt polypeptide (e.g., a human biologically active Wnt polypeptide). In some cases, the Wnt polypeptide is a Wnt3A, Wnt5A, or Wnt10B polypeptide. In some cases, the Wnt polypeptide is a Wnt3A polypeptide. In some cases, the Wnt polypeptide is human Wnt3A polypeptide. In some cases, the Wnt3A polypeptide is a Wnt3A variant described herein, e.g., comprising a modification and/or a truncation.

In some instances, a chaperone described herein comprises a protein or fragments thereof that facilitates in the assembly or disassembly of a macromolecular structure. In some instances, a chaperone comprises a protein or fragments thereof that facilitates in secretion, expression, stability, and/or purification. As used herein in the context of Wnt polypeptides, a chaperone comprises a protein or fragments thereof that facilitates in secretion, expression, stability, and/or purification of Wnt polypeptides. Furthermore, as used herein in the context of Wnt polypeptides, a chaperone is a protein or fragments thereof that is co-expressed with a Wnt polypeptide in a cell from an engineered cell line. In such cases, the culture condition is a serum-free condition.

In some embodiments, a chaperone described herein comprises Frizzled, Wntless, Afamin, or Porcupine. In some instances, the chaperone comprises Frizzled. Frizzled is a family of G protein-coupled receptor proteins which serve as receptors in the Wnt signaling pathway. In some instances, there are ten members in this family, Frizzled-1 (FZD1), Frizzled-2 (FZD2), Frizzled-3 (FZD3), Frizzled-4 (FZD4), Frizzled-5 (FZD5), Frizzled-6 (FZD6), Frizzled-7 (FZD7), Frizzled-8 (FZD8), Frizzled-9 (FZD9), and Frizzled-10 (FZD10). In some instances, a Frizzled protein is co-expressed with a Wnt polypeptide, forming, e.g., a 1:1 complex. In some cases, a Frizzled protein selected from FZD1, FZD2, FZD3, FZD4, FZD5, FZD6, FZD7, FZD8, FZD9, and FZD10 is co-expressed with a Wnt polypeptide. In some instances, a Frizzled protein co-expressed with a Wnt polypeptide improves secretion of the Wnt polypeptide, stabilizes the Wnt polypeptide, and/or enhances expression of the Wnt polypeptide, relative to a Wnt polypeptide in the absence of the Frizzled protein. In some instances, the Wnt polypeptide is Wnt5A polypeptide, Wnt10B polypeptide, or Wnt3A polypeptide. In some cases, the Wnt polypeptide is Wnt3A polypeptide.

In some embodiments, a chaperone comprises Frizzled-8 (FZD8). Frizzled-8, encoded by the FZD8 gene, is a seven-transmembrane domain protein and a receptor for Wnt polypeptides. In some instances, FZD8 is co-expressed with a Wnt polypeptide. In some cases, the molar ratio of FZD8 to Wnt polypeptide is 1:4, 1:3, 1:2, 1:1, 2:1, 3:1, or 4:1. In some instances, the molar ratio of FZD8 to Wnt polypeptide is 1:4. In some instances, the molar ratio of FZD8 to Wnt polypeptide is 1:2. In some instances, the molar ratio of FZD8 to Wnt polypeptide is 1:1. In some instances, the molar ratio of FZD8 to Wnt polypeptide is 2:1. In some cases, the molar ratio of FZD8 to Wnt polypeptide is 4:1. In some instances, FZD8 co-expressed with a Wnt polypeptide improves secretion of the Wnt polypeptide, stabilizes the Wnt polypeptide, and enhances expression of the Wnt polypeptide, relative to a Wnt polypeptide in the absence of FZD8. In some instances, the Wnt polypeptide is Wnt5A polypeptide, Wnt10B polypeptide, or Wnt3A polypeptide. In some cases, the Wnt polypeptide is Wnt3A polypeptide.

In some instances, human Frizzled-8 (NCBI Reference Seq: NP_114072.1; SEQ ID NO: 4) comprises 694 amino acids in length. In some cases, Frizzled-8 comprises a 27 amino acid signal sequence, a 248 amino acid extracellular N-terminus, and an 89 amino acid C-terminus. In some cases, the N-terminus further comprises two putative N-linked glycosylation sites, a polyproline segment and a polyglycine segment. In addition, the N-terminus comprises a cysteine-rich domain (CRD) that is about 120 amino acids in length. The C-terminus of Frizzled-8 comprises a Thr-x-Val tripeptide, a Lys-Thr-x-x-x-Trp motif, and a polyglycine repeat of 25 amino acids in length. In some instances, human FZD8 is co-expressed with a Wnt polypeptide. In some cases, the molar ratio of human FZD8 to Wnt polypeptide is 1:4, 1:3, 1:2, 1:1, 2:1, 3:1, or 4:1. In some instances, the molar ratio of human FZD8 to Wnt polypeptide is 1:4. In some instances, the molar ratio of human FZD8 to Wnt polypeptide is 1:2. In some instances, the molar ratio of human FZD8 to Wnt polypeptide is 1:1. In some instances, the molar ratio of human FZD8 to Wnt polypeptide is 2:1. In some cases, the molar ratio of human FZD8 to Wnt polypeptide is 4:1. In some instances, human FZD8 co-expressed with a Wnt polypeptide improves secretion of the Wnt polypeptide, stabilizes the Wnt polypeptide, and enhances expression of the Wnt polypeptide, relative to a Wnt polypeptide in the absence of human FZD8. In some instances, the Wnt polypeptide is Wnt5A polypeptide, Wnt10B polypeptide, or Wnt3A polypeptide. In some cases, the Wnt polypeptide is Wnt3A polypeptide.

In some instances, a Frizzled-8 polypeptide described herein comprises about 70%, 75%, 80%, 85%, 90%, 95%, 96%, 97%, 98%, or 99% sequence identity to human Frizzled-8. In some cases, a Frizzled-8 polypeptide described herein comprises about 70%, 75%, 80%, 85%, 90%, 95%, 96%, 97%, 98%, or 99% sequence identity to SEQ ID NO: 4. In some instances, a Frizzled-8 polypeptide comprising about 70%, 75%, 80%, 85%, 90%, 95%, 96%, 97%, 98%, or 99% sequence identity to SEQ ID NO: 4 is co-expressed with a Wnt polypeptide. In some cases, the molar ratio of the Frizzled-8 polypeptide to Wnt polypeptide is, e.g., 1:4, 1:3, 1:2, 1:1, 2:1, 3:1, or 4:1. In some instances, the molar ratio of the Frizzled-8 polypeptide to Wnt polypeptide is 1:4. In some instances, the molar ratio of the Frizzled-8 polypeptide to Wnt polypeptide is 1:2. In some instances, the molar ratio of the Frizzled-8 polypeptide to Wnt polypeptide is 1:1. In some instances, the molar ratio of the Frizzled-8 polypeptide to Wnt polypeptide is 2:1. In some cases, the molar ratio of the Frizzled-8 polypeptide to Wnt polypeptide is 4:1. In some instances, the Frizzled-8 protein co-expressed with a Wnt polypeptide improves secretion of the Wnt polypeptide, stabilizes the Wnt polypeptide, and enhances expression of the Wnt polypeptide, relative to a Wnt polypeptide in the absence of the Frizzled-8 protein. In some instances, the Wnt polypeptide is Wnt5A polypeptide, Wnt10B polypeptide, or Wnt3A polypeptide. In some cases, the Wnt polypeptide is Wnt3A polypeptide.

In some embodiments, a chaperone described herein comprises a Frizzled-8 fusion protein. In some cases, the Frizzled-8 fusion protein comprises a truncated Frizzled-8 protein. In some instances, the truncated Frizzled-8 protein comprises a cysteine-rich region (CRD) of Frizzled-8. In some instances, the truncated Frizzled-8 protein comprises the region spanning amino acid residue 1 to amino acid residue 151 of SEQ ID NO: 4. In other instances, the truncated Frizzled-8 protein comprises the region spanning amino acid residue 1 to amino acid residue 172 of SEQ ID NO: 4.

In some instances, the Frizzled-8 fusion protein further comprises the Fc portion of an antibody. In some instances, the antibody is selected from IgA, IgD, IgE, IgG or IgM. In some cases, the antibody is IgG. In some cases, the Frizzled-8 fusion protein comprises a truncated Frizzled-8 protein (e.g., the CRD portion of Frizzled-8) and an IgG Fc portion.

In some cases, the truncated Frizzled-8 protein is covalently linked to the Fc portion directly. In other cases, the truncated Frizzled-8 protein is covalently linked to the Fc portion indirectly via a linker. In some instances, a linker comprises a series of glycines, alanines, or a combination thereof. In some instances, a linker comprises the amino acid sequence IEGRMD (SEQ ID NO: 6).

In some cases, the Frizzled-8 fusion protein comprises at least 80%, 85%, 90%, 95%, 96%, 97%, 98%, or 99% sequence identity to SEQ ID NO: 5. In some cases, the Frizzled-8 fusion protein comprises at least 80% sequence identity to SEQ ID NO: 5. In some cases, the Frizzled-8 fusion protein comprises at least 85% sequence identity to SEQ ID NO: 5. In some cases, the Frizzled-8 fusion protein comprises at least 90% sequence identity to SEQ ID NO: 5. In some cases, the Frizzled-8 fusion protein comprises at least 95% sequence identity to SEQ ID NO: 5. In some cases, the Frizzled-8 fusion protein comprises at least 96% sequence identity to SEQ ID NO: 5. In some cases, the Frizzled-8 fusion protein comprises at least 97% sequence identity to SEQ ID NO: 5. In some cases, the Frizzled-8 fusion protein comprises at least 98% sequence identity to SEQ ID NO: 5. In some cases, the Frizzled-8 fusion protein comprises at least 99% sequence identity to SEQ ID NO: 5. In some cases, the Frizzled-8 fusion protein comprises 100% sequence identity to SEQ ID NO: 5. In some cases, the Frizzled-8 fusion protein consists the sequence set forth in SEQ ID NO: 5.

In some instances, a Frizzled-8 polypeptide comprising at least 80%, 85%, 90%, 95%, 96%, 97%, 98%, or 99% sequence identity to SEQ ID NO: 5 is co-expressed with a Wnt polypeptide. In some cases, the molar ratio of the Frizzled-8 polypeptide to Wnt polypeptide is, e.g., 1:4, 1:3, 1:2, 1:1, 2:1, 3:1, or 4:1. In some instances, the molar ratio of the Frizzled-8 polypeptide to Wnt polypeptide is 1:4. In some instances, the molar ratio of the Frizzled-8 polypeptide to Wnt polypeptide is 1:2. In some instances, the molar ratio of the Frizzled-8 polypeptide to Wnt polypeptide is 1:1. In some instances, the molar ratio of the Frizzled-8 polypeptide to Wnt polypeptide is 2:1. In some cases, the molar ratio of the Frizzled-8 polypeptide to Wnt polypeptide is 4:1. In some instances, the Frizzled-8 protein co-expressed with a Wnt polypeptide improves secretion of the Wnt polypeptide, stabilizes the Wnt polypeptide, and enhances expression of the Wnt polypeptide, relative to a Wnt polypeptide in the absence of the Frizzled-8 protein. In some instances, the Wnt polypeptide is Wnt5A polypeptide, Wnt10B polypeptide, or Wnt3A polypeptide. In some cases, the Wnt polypeptide is Wnt3A polypeptide. In some cases, the Wnt3A polypeptide is a Wnt3A variant described herein, e.g., comprising a modification and/or a truncation.

In some cases, the Frizzled-8 fusion protein comprises at least 80%, 85%, 90%, 95%, 96%, 97%, 98%, or 99% sequence identity to SEQ ID NO: 18. In some cases, the Frizzled-8 fusion protein comprises at least 80% sequence identity to SEQ ID NO: 18. In some cases, the Frizzled-8 fusion protein comprises at least 85% sequence identity to SEQ ID NO: 18. In some cases, the Frizzled-8 fusion protein comprises at least 90% sequence identity to SEQ ID NO: 18. In some cases, the Frizzled-8 fusion protein comprises at least 95% sequence identity to SEQ ID NO: 18. In some cases, the Frizzled-8 fusion protein comprises at least 96% sequence identity to SEQ ID NO: 18. In some cases, the Frizzled-8 fusion protein comprises at least 97% sequence identity to SEQ ID NO: 18. In some cases, the Frizzled-8 fusion protein comprises at least 98% sequence identity to SEQ ID NO: 18. In some cases, the Frizzled-8 fusion protein comprises at least 99% sequence identity to SEQ ID NO: 18. In some cases, the Frizzled-8 fusion protein comprises 100% sequence identity to SEQ ID NO: 18. In some cases, the Frizzled-8 fusion protein consists the sequence set forth in SEQ ID NO: 18.

In some instances, a Frizzled-8 polypeptide comprising at least 80%, 85%, 90%, 95%, 96%, 97%, 98%, or 99% sequence identity to SEQ ID NO: 18 is co-expressed with a Wnt polypeptide. In some cases, the molar ratio of the Frizzled-8 polypeptide to Wnt polypeptide is, e.g., 1:4, 1:3, 1:2, 1:1, 2:1, 3:1, or 4:1. In some instances, the molar ratio of the Frizzled-8 polypeptide to Wnt polypeptide is 1:4. In some instances, the molar ratio of the Frizzled-8 polypeptide to Wnt polypeptide is 1:2. In some instances, the molar ratio of the Frizzled-8 polypeptide to Wnt polypeptide is 1:1. In some instances, the molar ratio of the Frizzled-8 polypeptide to Wnt polypeptide is 2:1. In some cases, the molar ratio of the Frizzled-8 polypeptide to Wnt polypeptide is 4:1. In some instances, the Frizzled-8 protein co-expressed with a Wnt polypeptide improves secretion of the Wnt polypeptide, stabilizes the Wnt polypeptide, and enhances expression of the Wnt polypeptide, relative to a Wnt polypeptide in the absence of the Frizzled-8 protein. In some instances, the Wnt polypeptide is Wnt5A polypeptide, Wnt10B polypeptide, or Wnt3A polypeptide. In some cases, the Wnt polypeptide is Wnt3A polypeptide. In some cases, the Wnt3A polypeptide is a Wnt3A variant described herein, e.g., comprising a modification and/or a truncation.

### Wntless

In some embodiments, the chaperone comprises Wntless. Wntless, also known as G protein-coupled receptor 177 (GPR177) or protein evenness interrupted homolog (EVI), is a multiple-pass transmembrane protein that acts as a chaperone for lipid modified Wnt proteins, which regulates Wnt expression, subcellular location, binding and organelle-specific association of Wnt proteins. Human Wntless is encoded by the Wntless WNT ligand secretion mediator (WLS) gene (also known as EVI, FLJ23091, mig-14, MRP, or Wntless homolog). In some instances, human Wntless comprises isoforms 1, 2, and 3.

In some instances, Wntless interacts with a Wnt polypeptide described herein. In some cases, Wntless selectively interacts with a biologically functional Wnt polypeptide described herein. In some cases, the biologically functional Wnt polypeptide is a lipid-modified Wnt polypeptide.

In some cases, Wntless co-expressed with a Wnt polypeptide enhances Wnt polypeptide expression, improves Wnt polypeptide secretion, and/or stabilizes Wnt polypeptide. In some cases, this is relative to a Wnt polypeptide in an equivalent cell in the absence of Wntless. In some instances, the Wnt polypeptide is Wnt5A polypeptide, Wnt10B polypeptide, or Wnt3A polypeptide. In some cases, the Wnt polypeptide is Wnt3A polypeptide.

In some cases, a Wntless polypeptide comprises at least 70%, 80%, 85%, 90%, 95%, 96%, 97%, 98%, or 99% sequence identity to SEQ ID NO: 7. In some cases, a Wntless polypeptide comprising at least 70%, 80%, 85%, 90%, 95%, 96%, 97%, 98%, or 99% sequence identity to SEQ ID NO: 7 is co-expressed with a Wnt polypeptide. In some cases, the molar ratio of the Wntless polypeptide to the Wnt polypeptide is, e.g., 1:4, 1:3, 1:2, 1:1, 2:1, 3:1, or 4:1. In some cases, the Wntless polypeptide co-expressed with a Wnt polypeptide enhances Wnt polypeptide expression, improves Wnt polypeptide secretion, and/or stabilizes Wnt polypeptide. In some cases, this is relative to a Wnt polypeptide in an equivalent cell in the absence of Wntless. In some instances, the Wnt polypeptide is Wnt5A polypeptide, Wnt10B polypeptide, or Wnt3A polypeptide. In some cases, the Wnt polypeptide is Wnt3A polypeptide. In some cases, the Wnt3A polypeptide is a Wnt3A variant described herein, e.g., comprising a modification and/or a truncation.

### Afamin

In some embodiments, the chaperone comprises Afamin. Afamin, a serum glycoprotein, is a member of the albumin gene family and is encoded by the AFM gene. In some instances, Afamin interacts with a Wnt polypeptide described herein. In some cases, Afamin selectively interacts with a biologically functional Wnt polypeptide described herein. In some cases, the biologically functional Wnt polypeptide is a lipid-modified Wnt polypeptide.

In some instances, Afamin co-expressed with a Wnt polypeptide enhances Wnt polypeptide expression, improves Wnt polypeptide secretion, and/or stabilizes Wnt polypeptide. In some cases, this is relative to a Wnt polypeptide in an equivalent cell in the absence of Afamin. In some instances, the Wnt polypeptide is Wnt5A polypeptide, Wnt10B polypeptide, or Wnt3A polypeptide. In some cases, the Wnt polypeptide is Wnt3A polypeptide.

In some instances, an Afamin polypeptide comprises at least 70%, 80%, 85%, 90%, 95%, 96%, 97%, 98%, or 99% sequence identity to SEQ ID NO: 8. In some cases, an Afamin polypeptide comprising at least 70%, 80%, 85%, 90%, 95%, 96%, 97%, 98%, or 99% sequence identity to SEQ ID NO: 8 is co-expressed with a Wnt polypeptide. In some cases, the molar ratio of Afamin to the Wnt polypeptide is, e.g., 1:4, 1:3, 1:2, 1:1, 2:1, 3:1, or 4:1. In some cases, the Afamin polypeptide co-expressed with a Wnt polypeptide enhances Wnt polypeptide expression, improves Wnt polypeptide secretion, and/or stabilizes Wnt polypeptide. In some cases, this is relative to a Wnt polypeptide in an equivalent cell in the absence of Afamin. In some instances, the Wnt polypeptide is Wnt5A polypeptide, Wnt10B polypeptide, or Wnt3A polypeptide. In some cases, the Wnt polypeptide is Wnt3A polypeptide. In some cases, the Wnt3A polypeptide is a Wnt3A variant described herein, e.g., comprising a modification and/or a truncation.

### Porcupine

In some embodiments, the chaperone comprises Porcupine. Porcupine, encoded by the gene PORCN (or porcupine homolog, PPN, MG61, probable protein-cysteine N-palmitoyltransferase, or protein-serine O-palmitoleoyltransferase porcupine), is a multipass transmembrane endoplasmic reticulum protein involved in the processing of Wnt proteins. In some instances, Porcupine further comprises five different isoforms (isoforms 1-5).

In some instances, Porcupine interacts with a Wnt polypeptide described herein. In some cases, Porcupine selectively interacts with a biologically functional Wnt polypeptide described herein. In some cases, the biologically functional Wnt polypeptide is a lipid-modified Wnt polypeptide.

In some instances, Porcupine is co-expressed with a Wnt polypeptide, e.g., to enhance Wnt polypeptide expression, to improve Wnt polypeptide secretion, and/or to stabilize Wnt polypeptide. In some cases, this is relative to a Wnt polypeptide in an equivalent cell in the absence of Porcupine. In some instances, the Wnt polypeptide is Wnt5A polypeptide, Wnt10B polypeptide, or Wnt3A polypeptide. In some cases, the Wnt polypeptide is Wnt3A polypeptide.

In some instances, a Porcupine polypeptide comprises at least 70%, 80%, 85%, 90%, 95%, 96%, 97%, 98%, or 99% sequence identity to SEQ ID NO: 9. In some cases, a Porcupine polypeptide comprising at least 70%, 80%, 85%, 90%, 95%, 96%, 97%, 98%, or 99% sequence identity to SEQ ID NO: 9 is co-expressed with a Wnt polypeptide. In some cases, the molar ratio of Porcupine to the Wnt polypeptide is, e.g., 1:4, 1:3, 1:2, 1:1, 2:1, 3:1, or 4:1. In some cases, the Porcupine polypeptide co-expressed with a Wnt polypeptide enhances Wnt polypeptide expression, improves Wnt polypeptide secretion, and/or stabilizes Wnt polypeptide. In some cases, this is relative to a Wnt polypeptide in an equivalent cell in the absence of Porcupine. In some instances, the Wnt polypeptide is Wnt5A polypeptide, Wnt10B polypeptide, or Wnt3A polypeptide. In some cases, the Wnt polypeptide is Wnt3A polypeptide. In some cases, the Wnt3A polypeptide is a Wnt3A variant described herein, e.g., comprising a modification and/or a truncation.

Methods of Processing Wnt Polypeptides Produced from Minimal Serum Conditions

In some embodiments, described herein are methods of harvesting a Wnt polypeptide (e.g., a Wnt polypeptide-chaperone complex) from a culture comprising a minimal serum condition, and subsequently purifying the Wnt polypeptide to generate an isolated Wnt polypeptide. In some embodiments, a stable Wnt polypeptide-chaperone complex is harvested and then processed to generate an active Wnt polypeptide. In some instances, the Wnt polypeptide from the stable Wnt polypeptide-chaperone complex is inactive but becomes active once the Wnt polypeptide dissociates from the Wnt polypeptide-chaperone complex.

In some embodiments, the method comprises coexpressing a Wnt polypeptide with a chaperone in a cell in a conditioned media to generate a Wnt polypeptide-chaperone complex, harvesting the Wnt polypeptide-chaperone complex from the conditioned media, introduce the Wnt polypeptide-chaperone complex to either a plurality of beads immobilized with a sulfonated polyaromatic compound or to an affinity chromatography column comprising a polypeptide that interacts with the Fc portion of an antibody to generate a processed Wnt polypeptide, and contacting the processed Wnt polypeptide with an aqueous solution of liposomes to generate the liposomal Wnt polypeptide.

In some embodiments, the method comprises (a) coexpressing a Wnt polypeptide with a chaperone in a cell in a conditioned media to generate a Wnt polypeptide-chaperone complex; (b) harvesting the Wnt polypeptide-chaperone complex from the conditioned media; (c) introducing the Wnt polypeptide-chaperone complex to a column immobilized with a sulfonated polyaromatic compound to generate an eluted Wnt polypeptide-chaperone complex; (d) processing the eluted Wnt polypeptide-chaperone complex through an affinity chromatography column comprising a polypeptide that interacts with the Fc portion of an antibody to generate a processed Wnt polypeptide; and (e) contacting the processed Wnt polypeptide with an aqueous solution of liposomes to generate the liposomal Wnt polypeptide

In some embodiments, also described herein is a method comprising (a) coexpressing a Wnt polypeptide with a chaperone in a cell in a conditioned media to generate a Wnt polypeptide-chaperone complex; (b) harvesting the Wnt polypeptide-chaperone complex from the conditioned media; (c) introducing the Wnt polypeptide-chaperone complex to an affinity chromatography column comprising a polypeptide that interacts with the Fc portion of an antibody to generate an eluted Wnt polypeptide-chaperone complex; (d) processing the eluted Wnt polypeptide-chaperone complex through a column immobilized with a sulfonated polyaromatic compound to generate a processed Wnt polypeptide; and (e) contacting the processed Wnt polypeptide with an aqueous solution of liposomes to generate the liposomal Wnt polypeptide.

In some embodiments, additionally described herein is a method of preparing a functionally active Wnt polypeptide, comprising: (a) incubating a plurality of Wnt polypeptide-chaperone complexes with a buffer comprising a sugar detergent to generate a mixture comprising a first Wnt composition comprising a functionally inactive Wnt polypeptide and a chaperone composition; (b) separating the first Wnt composition from the mixture with a column immobilized with a sulfonated polyaromatic compound to generate a second Wnt composition comprising the functionally active Wnt polypeptide and the sugar detergent; (c) optionally purifying the second Wnt composition with an affinity chromatography column comprising a polypeptide that interacts with the Fc portion of an antibody, a mixed mode column, a size exclusion chromatography column, or a combination thereof, at least once to generate a third Wnt composition; and (d) contacting the second Wnt composition or optionally the third Wnt composition with an aqueous solution of liposomes to generate a final Wnt composition comprising a functionally active Wnt polypeptide.

In some embodiments, further described herein is a method of preparing a functionally active Wnt polypeptide, comprising: (a) purifying the plurality of Wnt polypeptide-chaperone complexes on a first affinity chromatography column comprising a polypeptide that interacts with the Fc portion of an antibody to generate an eluted mixture of Wnt polypeptide-chaperone complexes; (b) incubating the eluted mixture of Wnt polypeptide-chaperone complexes with the buffer comprising a sugar detergent to generate the mixture comprising the first Wnt composition comprising a functionally inactive Wnt polypeptide and a chaperone composition; (c) separating the first Wnt composition from the mixture with a column immobilized with a sulfonated polyaromatic compound to generate the second Wnt composition comprising the functionally active Wnt polypeptide and the sugar detergent; (d) purifying the second Wnt composition in tandem with a second affinity chromatography column comprising a polypeptide that interacts with the Fc portion of an antibody, a mixed mode column, and a size exclusion chromatography column to generate the third Wnt composition; and (e) contacting the third Wnt composition with an aqueous solution of liposomes to generate the final Wnt composition comprising a functionally active Wnt polypeptide.

In some instances, a non-limiting example of a sulfonated polyaromatic compound is Cibacron blue F3GA. In some instances, Cibacron blue F3GA is a triazinyl dye. In some instances, beads immobilized with a triazinyl dye is used in a purification step described supra. In some instances, a non-limiting example of a chromatographic column immobilized with Cibacron blue F3GA is a Blue Sepharose column.

In some embodiments, purification is carried out in batch mode with the use of a plurality of beads immobilized with a sulfonated polyaromatic compound. In general, the Wnt polypeptide (e.g., the Wnt polypeptide-chaperone complex) is bound to the sulfonated polyaromatic compound immobilized beads in a binding buffer containing a low concentration of salt. High salt destabilizes the non-covalent ionic interactions between the protein and the beads, thereby allow elution of the Wnt polypeptide (e.g., the Wnt polypeptide-chaperone complex). In some embodiments, the concentration of the salt used in the binding buffer is at most 0, 0.01, 5, 10, 15, 20, 25, 30, 40, 50 mM, or less. In some embodiments, the concentration of the salt used in the binding buffer is at least 0, 0.01, 5, 10, 15, 20, 25, 30, 40, 50 mM, or more. In some embodiments, one or more wash buffers are used to remove unbound impurities. In some embodiments, at most 1, 2, 3, 4, 5, or more wash steps are used. In some embodiments, at least 1, 2, 3, 4, 5, or less wash steps are used. In some embodiments, the concentration of the salt used in the wash buffer is at least 30, 40, 50, 60, 70, 80, 90, 100 mM, more. In some embodiments, the concentration of the salt used in the wash buffer is at most 30, 40, 50, 60, 70, 80, 90, 100 mM, less. In some embodiments, one or more elution steps follow. In some embodiments, the concentration of the salt in the elution buffer is at least 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000 mM, or more. In some embodiments, the concentration of the salt in the elution buffer is at most 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000 mM, or less. Exemplary salts include sodium chloride, potassium chloride, magnesium chloride, calcium chloride, calcium phosphate, potassium phosphate, magnesium phosphate, sodium phosphate, ammonium sulfate, ammonium chloride, ammonium phosphate, and the like. In some instances, the pH of a buffer described herein (e.g., a binding buffer, a wash buffer, and/or an elution buffer) is at least 4, 4.5, 5, 5.5, 6, 6.5, 7, 7.5, 8, 8.5, 9, or more. In some instances, the pH of the buffer is at most 4, 4.5, 5, 5.5, 6, 6.5, 7, 7.5, 8, 8.5, 9, or less. In some embodiments, the Wnt polypeptide is Wnt3A polypeptide, Wnt5A polypeptide, or Wnt10B polypeptide. In some embodiments, the Wnt polypeptide is Wnt3A polypeptide. In some cases, the Wnt3A polypeptide is a Wnt3A variant described herein, e.g., comprising a modification and/or a truncation.

In some embodiments, purification is carried out using a column immobilized with a sulfonated polyaromatic compound. In general, the Wnt polypeptide (e.g., the Wnt polypeptide-chaperone complex) is bound to the column immobilized with the sulfonated polyaromatic compound in a binding buffer containing a low concentration of salt. High salt destabilizes the non-covalent ionic interactions between the protein and the column beads, thereby allow elution of the Wnt polypeptide (e.g., the Wnt polypeptide-chaperone complex). In some embodiments, the concentration of the salt used in the binding buffer is at most 0, 0.01, 5, 10, 15, 20, 25, 30, 40, 50 mM, or less. In some embodiments, the concentration of the salt used in the binding buffer is at least 0, 0.01, 5, 10, 15, 20, 25, 30, 40, 50 mM, or more. In some embodiments, one or more wash buffers are used to remove unbound impurities. In some embodiments, at most 1, 2, 3, 4, 5, or more wash steps are used. In some embodiments, at least 1, 2, 3, 4, 5, or less wash steps are used. In some embodiments, the concentration of the salt used in the wash buffer is at least 30, 40, 50, 60, 70, 80, 90, 100 mM, more. In some embodiments, the concentration of the salt used in the wash buffer is at most 30, 40, 50, 60, 70, 80, 90, 100 mM, less. In some embodiments, one or more elution steps follow. In some embodiments, the concentration of the salt in the elution buffer is at least 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000 mM, or more. In some embodiments, the concentration of the salt in the elution buffer is at most 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000 mM, or less. Exemplary salts include sodium chloride, potassium chloride, magnesium chloride, calcium chloride, calcium phosphate, potassium phosphate, magnesium phosphate, sodium phosphate, ammonium sulfate, ammonium chloride, ammonium phosphate, and the like. In some instances, the pH of a buffer described herein (e.g., a binding buffer, a wash buffer, and/or an elution buffer) is at least 4, 4.5, 5, 5.5, 6, 6.5, 7, 7.5, 8, 8.5, 9, or more. In some instances, the pH of the buffer is at most 4, 4.5, 5, 5.5, 6, 6.5, 7, 7.5, 8, 8.5, 9, or less. In some embodiments, the Wnt polypeptide is Wnt3A polypeptide, Wnt5A polypeptide, or Wnt10B polypeptide. In some embodiments, the Wnt polypeptide is Wnt3A polypeptide. In some cases, the Wnt3A polypeptide is a Wnt3A variant described herein, e.g., comprising a modification and/or a truncation.

In some embodiments, purification of a Wnt polypeptide described herein with an affinity chromatography is carried out either in batch mode or using a column, and employs, for example, various immobilized beads for purification of a tag described herein. As discussed above, one or more tags contemplated herein include: poly-histidine tag, PA-tag, FLAG tag, human influenza hemagglutinin (HA) tag, Myc tag, glutathione-S transferase (GST), calmodulin binding protein (CBP), maltose-binding protein (MBP), ABDz1-tag (albumin), HaloTag®, heparin-binding peptide (HB) tag, poly-Arg tag, poly-Lys tag, S-tag, Strep-II tag, and SUMO tag.

In some cases, the affinity chromatography method is an antibody-based purification method. For example, in such cases, a plurality of beads is immobilized with a polypeptide that recognizes the Fc portion of an antibody (e.g., Protein A). In general, the Wnt polypeptide, e.g., the Wnt polypeptide-chaperone complex, and specifically the chaperone, is bound to the column immobilized with, for example, a Protein A polypeptide in a binding buffer at a pH of about 6.5 or higher (e.g., at a pH of about 6.8, 7, 7.2, 7.5, 7.7, 7.8, 8, 8.5, or higher). In some cases, an elution buffer for use with an affinity chromatography comprising a Protein A polypeptide comprises an acidic pH and is used to elute the Wnt polypeptide. In some cases, the elution buffer comprises a pH of about 2, 2.5, 3. 3.5, 4, 5 or about 6. In some cases, the elution buffer comprises a pH of about 3. In some instances, the elution step comprises a stepwise pH gradient. In some cases, the stepwise pH gradient comprises a decrease in pH, of from about 6 to about 3. In some cases, the decrease in pH is: about 6, about 5, about 4, about 3.5, and about 3. In some cases, the eluted fraction comprising the Wnt polypeptide is further neutralized by a Tris-HCl buffer. In some cases, the Tris-HCl buffer comprises a pH of about 9.5, and at a 1M concentration. In some instances, the Wnt polypeptide is Wnt5A polypeptide, Wnt10B polypeptide, or Wnt3A polypeptide. In some cases, the Wnt polypeptide is Wnt3A polypeptide. In some cases, the Wnt3A polypeptide is a Wnt3A variant described herein, e.g., comprising a modification and/or a truncation.

In some embodiments, a mixed mode chromatography column is utilized for the purification of a Wnt polypeptide described herein. Mixed mode chromatography (MMC) describes a chromatographic method that utilizes two or more different forms of interaction between the stationary phase and an analyte to achieve their separation. In some instances, mixed mode chromatography method is further divided into two subtypes, physical MMC and chemical MMC. The physical MMC method utilizes a stationary phase that comprises two or more types of packing materials, either in two different columns as a “tandem column”, in two opposing ends of the same column as in a “biphasic column”, or in a homogenized phase in a single column as in a “mixed-bed column”. The chemical MMC method utilizes one type of packing materials that contains two or more functionalities. For example, the chemical MMC may utilize hydrophobic and/or hydrophilic interactions with ion-exchange interactions to increase selectivity during purification. Exemplary types of chemical MMC include, but are not limited to, anion-exchange/reversed-phase (AEX/RP), cation-exchange/reversed-phase (CEX/RP), anion-exchanged/cation-exchange/reversed-phase (AEX/CEX/RP), AEX/HILIC, CEX/HILIC, and AEX/CEX/HILIC. Exemplary MMC columns include, but are not limited to, Acclaim Trinity P1 LC columns (ThermoFisher), Acclaim Mixed Mode WCX-1 LC columns (ThermoFisher), Acclaim Mixed Mode HILIC-1 LC columns (ThermoFisher), OmniPac PAX and PCX series of HPLC columns (ThermoFisher), and Bio-Gel® HT column (Bio-Rad).

In some embodiments, a mixed mode chromatography column is utilized for the purification of a Wnt polypeptide. In some instances, a physical MMC column is utilized for the purification of a Wnt polypeptide. In other instances, a chemical MMC column is utilized for the purification of a Wnt polypeptide. In some cases, the Wnt polypeptide is Wnt3A polypeptide, Wnt5A polypeptide, or Wnt10B polypeptide. In some cases, the Wnt polypeptide is Wnt3A polypeptide. In some cases, the Wnt3A polypeptide is a Wnt3A variant described herein, e.g., comprising a modification and/or a truncation.

In some embodiments, a size exclusion chromatography (SEC) column is utilized for the purification of a Wnt polypeptide described herein. Size-exclusion chromatography, also known as molecular sieve chromatography, separates molecules in solution based on their size and in some cases, based on their molecular weight. Exemplary SEC columns include, but are not limited to, silica-based columns such as TSKgel® SW-type columns (Sigma-Aldrich); and polymethacrylate-based columns such as TSKgel PW-type columns (Sigma-Aldrich).

In some embodiments, a size exclusion chromatography (SEC) column is utilized for the purification of a Wnt polypeptide. In some instances, a silica-based SEC column is utilized for the purification of a Wnt polypeptide. In other instances, a polymethacrylate-based SEC column is utilized for the purification of a Wnt polypeptide. In some cases, the Wnt polypeptide is Wnt3A polypeptide, Wnt5A polypeptide, or Wnt10B polypeptide. In some cases, the Wnt polypeptide is Wnt3A polypeptide. In some cases, the Wnt3A polypeptide is a Wnt3A variant described herein, e.g., comprising a modification and/or a truncation.

In some embodiments, a detergent is formulated into a binding buffer, a wash buffer, and/or an elution buffer described above. Exemplary detergents include anionic detergents such as alkylbenzenesulfonates, carboxylates, sulphonates, petroleum sulphonates, alkylbenzenesulphonates, naphthalenesulphonates, olefin sulphonates, alkyl sulphates, sulphates, sulphated natural oils and fats, sulphated esters, and sulphated alkanolamides; cationic detergents such as quaternary ammonium salts, amines with amide linkages, polyoxyethylene alkyl and alicyclic amines, n,n,n′,n′ tetrakis substituted ethylenediamines, and 2-alkyl 1-hydroxethyl 2-imidazolines; nonionic detergents such as poyoxyethylene (e.g., Tween, Triton, and the Brij series of detergents) and sugar detergents (e.g., octyl thioglucoside and maltosides); and amphoteric or zwitterionic detergents such as CHAPS. In some instances, the detergent stabilizes a Wnt polypeptide described herein. In some instances, the detergent acts as a competitive antagonist by competing against a Wnt polypeptide for binding with a chaperone (e.g., a Frizzled fusion protein).

In some embodiments, the detergent is a sugar detergent. In some cases, the sugar detergent is a glucoside detergent. In other cases, the detergent is a maltoside detergent. Exemplary glucoside detergent include, but are not limited to, n-hexyl-β-D-glucopyranoside, n-heptyl-β-D-glucopyranoside, n-octyl-β-D-glucopyranoside, n-octyl-α-D-glucopyranoside, octyl β-D-1-thioglucopyranoside, n-octyl-β-D-galactopyranoside, n-nonyl-β-D-glucopyranoside, n-decyl-β-D-glucopyranoside, n-dodecyl-β-D-glucopyranoside, and methyl-6-O—(N-heptylcarbamoyl)-α-D-glucopyranoside. Exemplary maltoside detergents include, but are not limited to, n-decyl-β-D-maltopyranoside, n-dodecyl-β-D-maltopyranoside, and 6-cyclohexyl-1-hexyl-β-D-maltopyranoside.

In some embodiments, a buffer, such as a binding buffer, wash buffer, and/or an elution buffer described above comprises a sugar detergent. In some cases, the buffer (e.g., a binding buffer, wash buffer, and/or an elution buffer) comprises a glucoside detergent. In such cases, the buffer (e.g., a binding buffer, wash buffer, and/or an elution buffer) comprises n-hexyl-β-D-glucopyranoside, n-heptyl-β-D-glucopyranoside, n-octyl-β-D-glucopyranoside, n-octyl-α-D-glucopyranoside, octyl β-D-1-thioglucopyranoside, n-octyl-β-D-galactopyranoside, n-nonyl-β-D-glucopyranoside, n-decyl-β-D-glucopyranoside, n-dodecyl-β-D-glucopyranoside, or methyl-6-O—(N-heptylcarbamoyl)-α-D-glucopyranoside. In some cases, the buffer comprises n-octyl-β-D-glucopyranoside or octyl β-D-1-thioglucopyranoside. In one embodiment, the buffer comprises n-octyl-β-D-glucopyranoside (also known as n-Octyl glucoside, OGP, OG, C8Glc, octyl-beta-glucopyranoside, or octyl-beta-D-glucopyranoside). In another embodiment, the buffer comprises octyl β-D-1-thioglucopyranoside (also known as octyl thioglucoside or OTG).

In some embodiments, a buffer (e.g., a binding buffer, wash buffer, and/or an elution buffer) comprises a maltoside detergent. In such cases, the buffer (e.g., a binding buffer, wash buffer, and/or an elution buffer) comprises n-decyl-β-D-maltopyranoside, n-dodecyl-β-D-maltopyranoside, or 6-cyclohexyl-1-hexyl-β-D-maltopyranoside.

In some embodiments, the concentration of the sugar detergent in a buffer described herein is from about 0.05% to about 5% w/v (weight by volume). In some instances, the concentration of the sugar detergent in the buffer is from about 0.1% to about 5%, from about 0.5% to about 4%, from about 1% to about 3%, from about 2% to about 5%, or from 3% to about 5% w/v. In some cases, the concentration of the sugar detergent in the buffer is about 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, or about 5% w/v. In some cases, the concentration of the sugar detergent in the buffer is about 0.1% w/v. In some cases, the concentration of the sugar detergent in the buffer is about 0.5% w/v. In some cases, the concentration of the sugar detergent in the buffer is about 1% w/v. In some cases, the concentration of the sugar detergent in the buffer is about 1.5% w/v. In some cases, the concentration of the sugar detergent in the buffer is about 2% w/v. In some cases, the concentration of the sugar detergent in the buffer is about 2.5% w/v. In some cases, the concentration of the sugar detergent in the buffer is about 3% w/v. In some cases, the concentration of the sugar detergent in the buffer is about 4% w/v. In some cases, the concentration of the sugar detergent in the buffer is about 5% w/v. In some cases, the buffer is an acetate-based buffer (e.g., comprises a concentration of about 10 mM, 20 mM, 30 mM, 50 mM, or more). In some instances, the buffer exhibits a pH of about 5, 5.5, 6, 6.5, or 7.

In some embodiments, the sugar detergent is a glucoside detergent. In some instances, the concentration of the glucoside detergent in the buffer is from about 0.05% to about 5%, about 0.1% to about 5%, from about 0.5% to about 4%, from about 1% to about 3%, from about 2% to about 5%, or from 3% to about 5% w/v. In some cases, the concentration of the glucoside detergent in the buffer is about 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, or about 5% w/v. In some cases, the concentration of the glucoside detergent in the buffer is about 0.1% w/v. In some cases, the concentration of the glucoside detergent in the buffer is about 0.5% w/v. In some cases, the concentration of the glucoside detergent in the buffer is about 1% w/v. In some cases, the concentration of the glucoside detergent in the buffer is about 1.5% w/v. In some cases, the concentration of the glucoside detergent in the buffer is about 2% w/v. In some cases, the concentration of the glucoside detergent in the buffer is about 2.5% w/v. In some cases, the concentration of the glucoside detergent in the buffer is about 3% w/v. In some cases, the concentration of the glucoside detergent in the buffer is about 4% w/v. In some cases, the concentration of the glucoside detergent in the buffer is about 5% w/v. In some cases, the buffer is an acetate-based buffer (e.g., comprises a concentration of about 10 mM, 20 mM, 30 mM, 50 mM, or more). In some instances, the buffer exhibits a pH of about 5, 5.5, 6, 6.5, or 7.

In some embodiments, the sugar detergent is n-octyl-β-D-glucopyranoside. In some instances, the concentration of n-octyl-β-D-glucopyranoside in the buffer is from about 0.05% to about 5%, about 0.1% to about 5%, from about 0.5% to about 4%, from about 1% to about 3%, from about 2% to about 5%, or from 3% to about 5% w/v. In some cases, the concentration of n-octyl-β-D-glucopyranoside in the buffer is about 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, or about 5% w/v. In some cases, the concentration of n-octyl-β-D-glucopyranoside in the buffer is about 0.1% w/v. In some cases, the concentration of n-octyl-β-D-glucopyranoside in the buffer is about 0.5% w/v. In some cases, the concentration of n-octyl-β-D-glucopyranoside in the buffer is about 1% w/v. In some cases, the concentration of n-octyl-β-D-glucopyranoside in the buffer is about 1.5% w/v. In some cases, the concentration of n-octyl-β-D-glucopyranoside in the buffer is about 2% w/v. In some cases, the concentration of n-octyl-β-D-glucopyranoside in the buffer is about 2.5% w/v. In some cases, the concentration of n-octyl-β-D-glucopyranoside in the buffer is about 3% w/v. In some cases, the concentration of n-octyl-β-D-glucopyranoside in the buffer is about 4% w/v. In some cases, the concentration of n-octyl-β-D-glucopyranoside in the buffer is about 5% w/v. In some cases, the buffer is an acetate-based buffer (e.g., comprises a concentration of about 10 mM, 20 mM, 30 mM, 50 mM, or more). In some instances, the buffer exhibits a pH of about 5, 5.5, 6, 6.5, or 7.

In some embodiments, the sugar detergent is octyl β-D-1-thioglucopyranoside. In some instances, the concentration of octyl β-D-1-thioglucopyranoside in the buffer is from about 0.05% to about 5%, about 0.1% to about 5%, from about 0.5% to about 4%, from about 1% to about 3%, from about 2% to about 5%, or from 3% to about 5% w/v. In some cases, the concentration of octyl β-D-1-thioglucopyranoside in the buffer is about 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, or about 5% w/v. In some cases, the concentration of octyl β-D-1-thioglucopyranoside in the buffer is about 0.1% w/v. In some cases, the concentration of octyl β-D-1-thioglucopyranoside in the buffer is about 0.5% w/v. In some cases, the concentration of octyl β-D-1-thioglucopyranoside in the buffer is about 1% w/v. In some cases, the concentration of octyl β-D-1-thioglucopyranoside in the buffer is about 1.5% w/v. In some cases, the concentration of octyl β-D-1-thioglucopyranoside in the buffer is about 2% w/v. In some cases, the concentration of octyl β-D-1-thioglucopyranoside in the buffer is about 2.5% w/v. In some cases, the concentration of octyl β-D-1-thioglucopyranoside in the buffer is about 3% w/v. In some cases, the concentration of octyl β-D-1-thioglucopyranoside in the buffer is about 4% w/v. In some cases, the concentration of octyl β-D-1-thioglucopyranoside in the buffer is about 5% w/v. In some cases, the buffer is an acetate-based buffer (e.g., comprises a concentration of about 10 mM, 20 mM, 30 mM, 50 mM, or more). In some instances, the buffer exhibits a pH of about 5, 5.5, 6, 6.5, or 7.

In some embodiments, the sugar detergent is a maltoside detergent. In some instances, the concentration of the maltoside detergent in the buffer is from about 0.05% to about 5%, about 0.1% to about 5%, from about 0.5% to about 4%, from about 1% to about 3%, from about 2% to about 5%, or from 3% to about 5% w/v. In some cases, the concentration of the maltoside detergent in the buffer is about 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, or about 5% w/v. In some cases, the concentration of the maltoside detergent in the buffer is about 0.1% w/v. In some cases, the concentration of the maltoside detergent in the buffer is about 0.5% w/v. In some cases, the concentration of the maltoside detergent in the buffer is about 1% w/v. In some cases, the concentration of the maltoside detergent in the buffer is about 1.5% w/v. In some cases, the concentration of the maltoside detergent in the buffer is about 2% w/v. In some cases, the concentration of the maltoside detergent in the buffer is about 2.5% w/v. In some cases, the concentration of the maltoside detergent in the buffer is about 3% w/v. In some cases, the concentration of the maltoside detergent in the buffer is about 4% w/v. In some cases, the concentration of the maltoside detergent in the buffer is about 5% w/v. In some cases, the buffer is an acetate-based buffer (e.g., comprises a concentration of about 10 mM, 20 mM, 30 mM, 50 mM, or more). In some instances, the buffer exhibits a pH of about 5, 5.5, 6, 6.5, or 7.

In some embodiments, the detergent is CHAPS, Triton X-100, or polysorbate 80. In some embodiments, the percentage of CHAPS, Triton X-100, or polysorbate 80 is at least 0.01%, 0.1%, 0.5%, 1%, 1.5%, 2%, 2.5%, 3%, 3.5%, 4%, 4.5%, 5%, or more. In some embodiments, the percentage of CHAPS, Triton X-100, or polysorbate 80 is at most 0.01%, 0.1%, 0.5%, 1%, 1.5%, 2%, 2.5%, 3%, 3.5%, 4%, 4.5%, 5%, or less. In some cases, the percentage of the detergent is a weight by volume (w/v) percentage.

In some instances, buffer components such as tris(hydroxymethyl)methylamine HCl (Tris-HCl), 3-{[tris(hydroxymethyl)methyl]amino}propanesulfonic acid (TAPS), N,N-bis(2-hydroxyethyl)glycine (Bicine), N-tris(hydroxymethyl)methylglycine (Tricine), 3-[N-Tris(hydroxymethyl)methylamino]-2-hydroxypropanesulfonic acid (TAPSO), 4-2-hydroxyethyl-1-piperazineethanesulfonic acid (HEPES), 3-(N-morpholino)propanesulfonic acid (MOPS), piperazine-N,N′-bis(2-ethanesulfonic acid) (PIPES), 2-(N-morpholino)ethanesulfonic acid (MES), and the like, are used. In some instances, the pH of the buffer is at least 4, 4.5, 5, 5.5, 6, 6.5, 7, 7.5, 8, 8.5, 9, or more. In some instances, the pH of the buffer is at most 4, 4.5, 5, 5.5, 6, 6.5, 7, 7.5, 8, 8.5, 9, or less.

In some instances, a basic amino acid is formulated into a binding buffer, a wash buffer, and/or an elution buffer described above. Exemplary basic amino acids comprise histidine, arginine, lysine, hydroxylysine, ornithine, and citrulline. In some instances, a basic amino acid selected from: histidine, arginine, lysine, hydroxylysine, ornithine, or citrulline is formulated into a binding buffer, a wash buffer, and/or an elution buffer described above. In some cases, the concentration of the basic amino acid in the binding buffer, wash buffer, and/or elution buffer is from about 0.1M to about 2M (e.g., from about 0.1M to about 1.5M, from about 0.1M to about 1M, from about 0.1M to about 0.5M, from about 0.2M to about 1.5M, from about 0.2M to about 1M, from about 0.3M to about 1M, or from about 0.3M to about 0.5M).

In some cases, the basic amino acid is arginine. In some cases, the concentration of arginine in the binding buffer, wash buffer, and/or elution buffer is from about 0.1M to about 2M. In some cases, the concentration of arginine in the elution buffer is from about 0.1M to about 2 M. In some cases, the concentration of arginine in the elution buffer is from about 0.1M to about 1.5M, from about 0.1M to about 1M, from about 0.1M to about 0.5M, from about 0.2M to about 1.5M, from about 0.2M to about 1M, from about 0.3M to about 1M, or from about 0.3M to about 0.5M. In some cases, the concentration of arginine in the elution buffer is from about 0.1M to about 0.5M. In some cases, the concentration of arginine in the elution buffer is about 0.1M, 0.2M, 0.3M, 0.4M, 0.5M, 0.6M, 0.7M, 0.8M, 0.9M, 1M, or about 1.5M.

In some instances, an elution buffer for a mixed mode chromatography column comprises from about 0.1M to about 2 M concentration of arginine. In some cases, the elution buffer comprises from about 0.1M to about 1.5M, from about 0.1M to about 1M, from about 0.1M to about 0.5M, from about 0.2M to about 1.5M, from about 0.2M to about 1M, from about 0.3M to about 1M, or from about 0.3M to about 0.5M concentration of arginine. In some cases, the elution buffer comprises from about 0.1M to about 0.5M concentration of arginine. In some cases, the elution buffer comprises about 0.1M, 0.2M, 0.3M, 0.4M, 0.5M, 0.6M, 0.7M, 0.8M, 0.9M, 1M, or about 1.5M concentration of arginine.

In some embodiments, a purification strategy comprises a first step in which a solution (e.g., a conditioned media) comprising a Wnt polypeptide-chaperone complex is loaded onto a first affinity chromatography column comprising a polypeptide that interacts with the Fc portion of an antibody to generate an eluted mixture of Wnt polypeptide-chaperone complexes. In some instances, the eluate from the first affinity chromatography column is further incubated in a buffer solution comprising a sugar detergent (e.g., a glucoside detergent such as n-octyl-β-D-glucopyranoside or octyl β-D-1-thioglucopyranoside). In some cases, the concentration of the sugar detergent (e.g., a glucoside detergent such as n-octyl-β-D-glucopyranoside or octyl β-D-1-thioglucopyranoside) in the buffer solution is about 0.1%, 0.5%, 1%, 1.5%, or about 2% w/v; or about 1% w/v. In some instances, the eluate is then loaded onto a column immobilized with a sulfonated polyaromatic compound to generate the second Wnt composition comprising the functionally active Wnt polypeptide and the sugar detergent, e.g., to remove the chaperone (e.g., Frizzled-8 fusion proteins) from the second Wnt composition. In some cases, the elution buffer for the column immobilized with a sulfonated polyaromatic compound comprises a step gradient. In other cases, the elution buffer for the column immobilized with a sulfonated polyaromatic compound comprises a salt gradient from about 0.5M to about 2M salt, from about 0.6M to about 2M salt, or from about 0.8M to about 2M salt. In some instances, the second Wnt composition is further purified with a second affinity chromatography column comprising a polypeptide that interacts with the Fc portion of an antibody, a mixed mode column, a size exclusion chromatography column, or a combination thereof, to generate the third Wnt composition. In some instances, the second Wnt composition is further purified in tandem with a second affinity chromatography column comprising a polypeptide that interacts with the Fc portion of an antibody, followed by a mixed mode column, and finally a size exclusion chromatography column to generate the third Wnt composition. In some cases, the second affinity chromatography column removes residual chaperone (e.g., Frizzled-8 fusion proteins) from the second Wnt composition. In some cases, the mixed mode column removes Wnt polypeptide fragments from the second Wnt composition. In some cases, the size exclusion chromatography column removes residual Wnt polypeptide fragments from the second Wnt composition to generate the third Wnt composition.

In some embodiments, a purification strategy comprises a first step in which a solution (e.g., a conditioned media) comprising a Wnt polypeptide-chaperone complex is loaded onto a column immobilized with a sulfonated polyaromatic compound, followed by a second step in which the Wnt polypeptide (e.g., the Wnt polypeptide-chaperone complex) eluted from the first step is further processed on an affinity chromatography column to generate a purified Wnt polypeptide. In some cases, a detergent is further added to the solution comprising a Wnt polypeptide (e.g., a Wnt polypeptide-chaperone complex) prior to loading onto the column immobilized with a sulfonated polyaromatic compound. In some cases, the purified Wnt polypeptide is further processed with an aqueous solution of liposomes to generate a liposomal Wnt polypeptide.

In some embodiments, a purification strategy comprises a first step in which a solution (e.g., a conditioned media) comprising a Wnt polypeptide-chaperone complex is loaded on an affinity chromatography column followed by a second step which comprises a column immobilized with a sulfonated polyaromatic compound. In some cases, a detergent is added to an eluted Wnt polypeptide from the first step prior to loading the eluted Wnt polypeptide comprising the detergent onto the column immobilized with a sulfonated polyaromatic compound. In some cases, a purified Wnt polypeptide eluted from the column immobilized with a sulfonated polyaromatic compound is further processed with an aqueous solution of liposomes to generate a liposomal Wnt polypeptide.

In some embodiments, a purification strategy comprises harvesting a Wnt polypeptide-chaperone complex from a conditioned media and loading onto an affinity chromatography column. In some cases, the elute from the column is further processed with an aqueous solution of liposomes to generate a liposomal Wnt polypeptide.

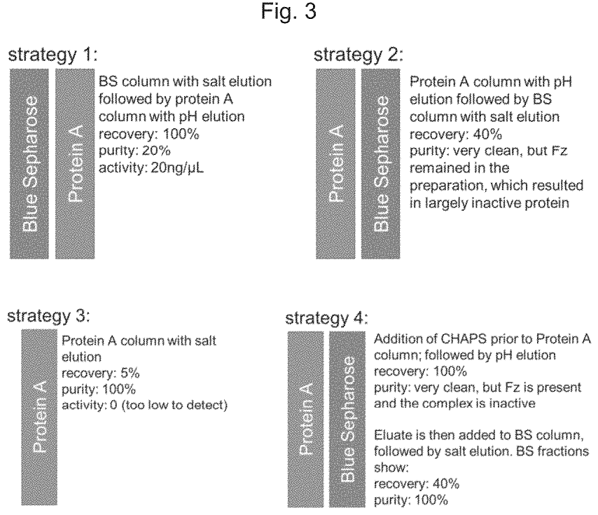

In some embodiments, a purification strategy illustrated in FIG. 3 is utilized for purification of a Wnt polypeptide described herein. In some instances, the Wnt polypeptide is Wnt5A polypeptide, Wnt10B polypeptide, or Wnt3A polypeptide. In some cases, the Wnt polypeptide is Wnt3A polypeptide. In some cases, the Wnt3A polypeptide is a Wnt3A variant described herein, e.g., comprising a modification and/or a truncation.

In some instances, the affinity of Wnt3A protein to its binding partners is at least about 1.1 nM, 1.3 nM, 1.5 nM, 1.7 nM, 2 nM, 2.3 nM, 2.5 nM, 2.7 nM, 3 nM, 3.1 nM, 3.2 nM, 3.3 nM, 3.4 nM, 3.5 nM, 3.6 nM, 3.7 nM, 3.8 nM, 3.9 nM, or more. In some instances, the affinity of Wnt3a protein to its binding partners is at most about 1.1 nM, 1.3 nM, 1.5 nM, 1.7 nM, 2 nM, 2.3 nM, 2.5 nM, 2.7 nM, 3 nM, 3.1 nM, 3.2 nM, 3.3 nM, 3.4 nM, 3.5 nM, 3.6 nM, 3.7 nM, 3.8 nM, 3.9 nM, or less.

In some embodiments, the concentration and yield of the eluted Wnt polypeptide is measured prior to subjecting to a further purification step. In some embodiments, the yield is at least about 20%, 30%, 40%, 50%, 60%, 70%, 80%, 90%, 95%, or more. In some embodiments, the yield is at most about 20%, 30%, 40%, 50%, 60%, 70%, 80%, 90%, 95%, or less. In some embodiments, the purity is at least about 20%, 30%, 40%, 50%, 60%, 70%, 80%, 90%, 95%, or more. In some embodiments, the purity is at most about 20%, 30%, 40%, 50%, 60%, 70%, 80%, 90%, 95%, or less.

In some embodiments the Wnt polypeptide (e.g., Wnt3A polypeptide) is purified to an initial concentration of at least about 5 μg/ml; usually at least about 10 μg/ml, more usually at least about 50 μg/ml, and may be present at greater than about 100 μg/ml.

In some embodiments, the isolated Wnt polypeptide (e.g., Wnt3A polypeptide) is further formulated in a liposome. In some cases, the Wnt polypeptide (e.g., Wnt3A polypeptide) is stabilized in a formulation with a detergent. In some cases, the Wnt polypeptide (e.g., Wnt3A polypeptide) is stabilized in a formulation with lipids.

In some embodiments, the liposome is fabricated using methods well known in the art. Liposomes are artificially-prepared spherical vesicles that compose a lamellar phase lipid bilayer and an aqueous core. There are several types of liposomes, such as the multilamellar vesicle (MLV), small unilamellar liposome vesicle (SUV), the large unilamellar vesicle (LUV), and the cochleate vesicle. In some instances, liposomes are formed by phospholipids. In some embodiments, phospholipids are separated into those with diacylglyceride structures or those derived from phosphosphingolipids. In some embodiments, the diacylglyceride structures include phosphatidic acid (phosphatidate) (PA), phosphatidylethanolamine (cephalin) (PE), phosphatidylcholine (lecithin) (PC), phosphatidylserine (PS), and phosphoinositides such as phosphatidylinositol (PI), phosphatidylinositol phosphate (PIP), phosphatidylinositol bisphosphate (PIP2), and phosphatidylinositol triphosphate (PIP3). In some embodiments, phosphosphingolipids include ceramide phosphorylcholine, ceramide phosphorylethanolamine, and ceramide phosphoryllipid. In some embodiments, the liposomes are formed from phosphatidylcholines.

In some embodiments, the lipids are also selected based on its transition phase temperature (Tₘ), or the temperature interface between the liquid crystalline phase and the gel phase. In some embodiments, the Tₘ is influenced by the head group species, hydrocarbon length, unsaturation, and the charge. For example, short lipids (lipids containing 8, 10, or 12 tail carbon chain length) have liquid crystalline phase at temperatures below 4° C. However, liposomes manufactured from these short chain carbon lipids are toxic to cells because they dissolve cell membranes. Liposomes manufactured from longer carbon-chain lipids are not toxic to cells, but their transition temperatures are higher. For example, 1,2-dipalmitoyl-sn-glycero-3-phosphocholine (DPPC) which has a 16 tail carbon length, has a T₁ of about 41° C. In some embodiments, the lipids used herein have a T₁ of between about 10° C. and about 37° C., 15° C. and about 30° C., 18° C. and about 27° C., or 21° C. and about 25° C. In some embodiments, the lipids used herein have a T₁ of at least 22° C., 23° C., 24° C., or more. In some embodiments, the lipids used herein have a T₁ of at most 22° C., 23° C., 24° C., or less. In some embodiments, the lipids used herein have a tail carbon length of at least about 12, 13, 14, or more. In some embodiments, the lipids used herein have a tail carbon length of at most about 12, 13, 14, or less.

In some embodiments, the lipids are further selected based on the net charge of the liposome. In some embodiments, the liposome has a net charge of 0 at a pH of between about 4.0 and about 10.0, about 5.0 and about 9.0, about 6.5 and about 8.0, about 7.0 and about 7.8, or about 7.2 and about 7.6. In some embodiments, the liposome has a net charge of 0 at a pH of about 7.3, about 7.4, or about 7.5. In some embodiments, the liposome has a net positive charge at a pH of between about 4.0 and about 10.0, about 5.0 and about 9.0, about 6.5 and about 8.0, about 7.0 and about 7.8, or about 7.2 and about 7.6. In some embodiments, the liposome has a net positive charge at a pH of about 7.3, about 7.4, or about 7.5. In some embodiments, the liposome has a net negative charge at a pH of between about 4.0 and about 10.0, about 5.0 and about 9.0, about 6.5 and about 8.0, about 7.0 and about 7.8, or about 7.2 and about 7.6. In some embodiments, the liposome has a net negative charge at a pH of about 7.3, about 7.4, or about 7.5.

In some embodiments, lipids are selected from 1,2-dimyristoyl-sn-glycero-3-phosphocholine (DMPC), 1,2-dipalmitoyl-sn-glycero-3-phosphocholine (DPPC), 1-tetradecanoyl-2-hexadecanoyl-sn-glycero-3-phosphocholine (MPPC), 1,2-dimyristoyl-sn-glycero-3-phospho-L-serine (DMPS), and 1,2-dihexanoyl-sn-glycero-3-phosphocholine (DMPG). In some embodiments, the lipid is DMPC.

In some embodiments, an additional lipid is fabricated into the liposome. In some embodiments, the additional lipid is cholesterol. In some instances, the concentration of a phosphatidylcholine such as DMPC and cholesterol is defined by a value such as a ratio. In some embodiments, the ratio of the concentrations of phosphatidylcholine such as DMPC and cholesterol is between about 50:50, about 55:45, about 60:40, about 65:35, about 70:30, about 75:25, about 80:20, about 85:15, about 90:10, about 95:5, about 99:1, or about 100:0. In some embodiments, the ratio of the concentrations of phosphatidylcholine such as DMPC and cholesterol is about 90:10. In some embodiments, the concentration unit is moles. In some embodiments, the ratio is mole:mole.

In some embodiments, the liposome is prepared with an ethanol injection-based method. In some instances, the method is as described in Wagner. et al. “The Crossflow Injection Technique: An improvement of the Ethanol Injection Method,” Journal of Liposome Research, 12(3): 259-270 (2002).

In some embodiments, the Wnt polypeptide is reconstituted with a liposome at a concentration of at least about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.4, 0.5, 0.6, 0.7, 0.8, 0.9, 1, 1.5, 2, 2.5, 3, 3.5, 4 ng/μL or more. In some embodiments, the Wnt polypeptide is reconstituted with a liposome at a concentration of at most about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.4, 0.5, 0.6, 0.7, 0.8, 0.9, 1, 1.5, 2, 2.5, 3, 3.5, 4 ng/μL or less. In some embodiments, the Wnt polypeptide is reconstituted with a liposome at a concentration of about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.4, 0.5, 0.6, 0.7, 0.8, 0.9, 1, 1.5, 2, 2.5, 3, 3.5, 4 ng/μL. In some embodiments, the Wnt polypeptide is Wnt3A polypeptide, Wnt5A polypeptide, or Wnt10b polypeptide. In some embodiments, the Wnt polypeptide is Wnt3A polypeptide.

In some embodiments, the Wnt polypeptide is reconstituted with a liposome at a ratio of at least about 0.1:50, 0.5:30, 1:20, or 1:14 Wnt polypeptide to liposome, or more. In some embodiments, the Wnt polypeptide is reconstituted with a liposome at a ratio of at most about 0.1:50, 0.5:30, 1:20, or 1:14 Wnt polypeptide to liposome, or less. In some instances, the ratio is a volume to volume ratio. In some instances, the unit of Wnt polypeptide is nanogram unit.

In some embodiments, the temperature at which the Wnt polypeptide is reconstituted with a liposome is at least between about 15° C. and about 37° C., about 18° C. and about 33° C., about 20° C. and about 30° C., about 25° C. and about 30° C., or about 20° C. and about 28° C. In some embodiments, the temperature is at least between about 15° C. and about 37° C. In some embodiments, the temperature is at least between about 18° C. and about 33° C. In some embodiments, the temperature is at least between about 20° C. and about 30° C. In some embodiments, the temperature is at least about 21° C., 22° C., 23° C., 24° C., 25° C., 26° C., 27° C., 28° C., 29° C., 30° C., or more. In some embodiments, the temperature is at most about 21° C., 22° C., 23° C., 24° C., 25° C., 26° C., 27° C., 28° C., 29° C., 30° C., or less. In some embodiments, the Wnt polypeptide is Wnt3A polypeptide, Wnt5A polypeptide, or Wnt10b polypeptide. In some embodiments, the Wnt polypeptide is Wnt3A polypeptide.

In some embodiments, the Wnt polypeptide is incubated with the liposome for at least 10 minutes, 20 minutes, 30 minutes, 1 hour, 1.5 hour, 2 hours, 2.5 hours, 3 hours, 4 hours, 5 hours, 6 hours, or more. In some instances, the Wnt polypeptide is incubated with the liposome for about 10 minutes, 20 minutes, 30 minutes, 1 hour, 1.5 hour, 2 hours, 2.5 hours, 3 hours, 4 hours, 5 hours, 6 hours, or more. In some instances, the Wnt polypeptide is incubated with the liposome for at least 30 minutes. In some instances, the Wnt polypeptide is incubated with the liposome for at least 1 hour. In some instances, the Wnt polypeptide is incubated with the liposome for at least 1.5 hour. In some instances, the Wnt polypeptide is incubated with the liposome for at least 2 hours. In some instances, the Wnt polypeptide is incubated with the liposome for at least 3 hours.

In some embodiments, the Wnt polypeptide is integrated into the liposomal membrane. In some cases, the Wnt polypeptide protrudes from the liposomal membrane onto the surface of the lipid membrane. In some instances, the Wnt polypeptide is not incorporated into the aqueous core of the liposome. In some embodiments, the Wnt polypeptide is Wnt3A polypeptide, Wnt5A polypeptide, or Wnt10B polypeptide. In some embodiments, the Wnt polypeptide is Wnt3A polypeptide. In some embodiments, the Wnt3A polypeptide is integrated into the liposomal membrane. In some cases, the Wnt3A polypeptide protrudes from the liposomal membrane onto the surface of the lipid membrane. In some instances, the Wnt3A polypeptide is not incorporated into the aqueous core of the liposome.

In some embodiments, the liposomal Wnt polypeptide has a liposomal particle size distribution of from about 10 nm to about 1 μm, from 10 nm to about 500 nm, from about 50 nm to about 300 nm, from about 50 nm to about 200 nm, from about 100 nm to about 500 nm, from about 100 nm to about 300 nm, or from about 100 nm to about 200 nm. In some instances, the liposomal Wnt polypeptide has a liposomal particle size distribution of from 10 nm to about 500 nm. In some instances, the liposomal Wnt polypeptide has a liposomal particle size distribution of from about 50 nm to about 300 nm. In some instances, the liposomal Wnt polypeptide has a liposomal particle size distribution of from about 50 nm to about 200 nm. In some instances, the liposomal Wnt polypeptide has a liposomal particle size distribution of from about 100 nm to about 200 nm. In some instances, the liposomal Wnt polypeptide has a liposomal particle size distribution of from about 150 nm to about 200 nm. In some instances, the liposomal Wnt polypeptide has a liposomal particle size distribution of from about 50 nm to about 150 nm.

In some embodiments, the liposomal Wnt polypeptide has a liposomal particle size distribution of less than about 1 μm, less than about 500 nm, less than about 300 nm, less than about 200 nm, or less than about 150 nm. In some instances, the liposomal Wnt polypeptide has a liposomal particle size distribution of less than about 1 μm. In some instances, the liposomal Wnt polypeptide has a liposomal particle size distribution of less than about 500 nm. In some instances, the liposomal Wnt polypeptide has a liposomal particle size distribution of less than about 300 nm. In some instances, the liposomal Wnt polypeptide has a liposomal particle size distribution of less than about 200 nm. In some instances, the liposomal Wnt polypeptide has a liposomal particle size distribution of less than about 170 nm. In some instances, the liposomal Wnt polypeptide has a liposomal particle size distribution of less than about 150 nm.

In some embodiments, the Wnt polypeptide reconstituted with a liposome is referred to as liposomal Wnt polypeptide or L-Wnt. In some embodiments, the Wnt polypeptide is Wnt3A polypeptide, Wnt5A polypeptide, or Wnt10B polypeptide. In some embodiments, the Wnt polypeptide is Wnt3A polypeptide. In some embodiments, the Wnt3A polypeptide reconstituted with a liposome is referred to as liposomal Wnt3A polypeptide or L-Wnt3A. In some embodiments, the Wnt polypeptide is Wnt5A polypeptide. In some embodiments, the Wnt5A polypeptide reconstituted with a liposome is referred to as liposomal Wnt5A polypeptide or L-Wnt5A. In some embodiments, the Wnt polypeptide is Wnt10B polypeptide. In some embodiments, the Wnt10B polypeptide reconstituted with a liposome is referred to as liposomal Wnt10B polypeptide or L-Wnt10B.

In some embodiments, the L-Wnt undergoes a centrifugation step and is then suspended in a buffer. Exemplary buffers include, but are not limited to, phosphate buffered saline (PBS) or a sucrose-based buffer such as a phosphate/sucrose buffer, a histidine/sucrose buffer, a citrate/sucrose buffer, an acetate/sucrose buffer, a sucrose/NaCl based buffer, a phosphate/sucrose/NaCl buffer, a histidine/sucrose/NaCl buffer, a citrate/sucrose/NaCl buffer, or an acetate/sucrose/NaCl buffer. In some instances, the sucrose-based buffer comprises from about 50 mM sucrose to about 500 mM sucrose. In some cases, the sucrose-based buffer comprises about 300 mM sucrose. In some instances, the phosphate/sucrose buffer comprises from about 5 mM phosphate to about 50 mM phosphate and from about 50 mM sucrose to about 500 mM sucrose. In some cases, the phosphate/sucrose buffer comprises about 10 mM phosphate and about 300 mM sucrose. In some cases, the histidine/sucrose buffer comprises about 10 mM histidine and about 300 mM sucrose. In some instances, the citrate/sucrose buffer comprises from about 5 mM citrate to about 50 mM citrate and from about 50 mM sucrose to about 500 mM sucrose. In some cases, the citrate/sucrose buffer comprises about 10 mM citrate and about 300 mM sucrose. In some instances, the acetate/sucrose buffer comprises from about 5 mM acetate to about 50 mM acetate and from about 50 mM sucrose to about 500 mM sucrose. In some cases, the acetate/sucrose buffer comprises about 10 mM acetate and about 300 mM sucrose. In some instances, the sucrose/NaCl-based buffer comprises from about 50 mM sucrose to about 300 mM sucrose and from about 5 mM NaCl to about 200 mM NaCl. In some cases, the sucrose/NaCl-based buffer comprises about 100 mM sucrose and 100 mM NaCl. In some instances, the phosphate/sucrose/NaCl buffer comprises from about 5 mM phosphate to about 50 mM phosphate, from about 50 mM sucrose to about 300 mM sucrose, and from about 5 mM NaCl to about 200 mM NaCl. In some cases, the phosphate/sucrose/NaCl buffer comprises about 10 mM phosphate, about 100 mM sucrose, and about 100 mM NaCl. In some instances, the histidine/sucrose/NaCl buffer comprises from about 5 mM histidine to about 50 mM histidine, from about 50 mM sucrose to about 300 mM sucrose, and from about 5 mM NaCl to about 200 mM NaCl. In some cases, the histidine/sucrose/NaCl buffer comprises about 10 mM histidine, about 100 mM sucrose, and about 100 mM NaCl. In some instances, the citrate/sucrose/NaCl buffer comprises from about 5 mM citrate to about 50 mM citrate, from about 50 mM sucrose to about 300 mM sucrose, and from about 5 mM NaCl to about 200 mM NaCl. In some cases, the citrate/sucrose/NaCl buffer comprises about 10 mM citrate, about 100 mM sucrose, and about 100 mM NaCl. In some instances, the acetate/sucrose/NaCl buffer comprises from about 5 mM acetate to about 50 mM acetate, from about 50 mM sucrose to about 300 mM sucrose, and from about 5 mM NaCl to about 200 mM NaCl. In some cases, the acetate/sucrose/NaCl buffer comprises about 10 mM acetate, about 100 mM sucrose, and about 100 mM NaCl.

In some embodiments, the L-Wnt undergoes a filtration step. In some instances, the filtration step comprises an ultrafiltration, a diafiltration, nanofiltration, steril filtration, or a combination thereof. Exemplary filtration membranes include, but are not limited to, cellulose acetate (CA), polysulfone (PS), polyether sulfone (PES), polyacrilonitrile (PAN), polyvinylidiene fluoride (PVDF), polypropylene (PP), polyethylene (PE), and polyvinyl chloride (PVC). In some instances, the L-Wnt undergoes one or more filtrations such as an ultrafiltration, a diafiltration, nanofiltration, a steril filtration, or a combination thereof. In some instances, L-Wnt undergoes an ultrafiltration and a nanofiltration for removal of one or more biological contaminant such as protein contaminants and microbial contaminants. In some instances, the nanofiltration removes one or more viral contaminants. In some instances, the L-Wnt further undergoes a diafiltration step for buffer exchange. Exemplary buffers include, but are not limited to, phosphate buffered saline (PBS) or a sucrose-based buffer such as a phosphate/sucrose buffer, a histidine/sucrose buffer, a citrate/sucrose buffer, an acetate/sucrose buffer, a sucrose/NaCl based buffer, a phosphate/sucrose/NaCl buffer, a histidine/sucrose/NaCl buffer, a citrate/sucrose/NaCl buffer, or an acetate/sucrose/NaCl buffer. In some instances, the sucrose-based buffer comprises from about 50 mM sucrose to about 500 mM sucrose. In some cases, the sucrose-based buffer comprises about 300 mM sucrose. In some instances, the phosphate/sucrose buffer comprises from about 5 mM phosphate to about 50 mM phosphate and from about 50 mM sucrose to about 500 mM sucrose. In some cases, the phosphate/sucrose buffer comprises about 10 mM phosphate and about 300 mM sucrose. In some cases, the histidine/sucrose buffer comprises about 10 mM histidine and about 300 mM sucrose. In some instances, the citrate/sucrose buffer comprises from about 5 mM citrate to about 50 mM citrate and from about 50 mM sucrose to about 500 mM sucrose. In some cases, the citrate/sucrose buffer comprises about 10 mM citrate and about 300 mM sucrose. In some instances, the acetate/sucrose buffer comprises from about 5 mM acetate to about 50 mM acetate and from about 50 mM sucrose to about 500 mM sucrose. In some cases, the acetate/sucrose buffer comprises about 10 mM acetate and about 300 mM sucrose. In some instances, the sucrose/NaCl-based buffer comprises from about 50 mM sucrose to about 300 mM sucrose and from about 5 mM NaCl to about 200 mM NaCl. In some cases, the sucrose/NaCl-based buffer comprises about 100 mM sucrose and 100 mM NaCl. In some instances, the phosphate/sucrose/NaCl buffer comprises from about 5 mM phosphate to about 50 mM phosphate, from about 50 mM sucrose to about 300 mM sucrose, and from about 5 mM NaCl to about 200 mM NaCl. In some cases, the phosphate/sucrose/NaCl buffer comprises about 10 mM phosphate, about 100 mM sucrose, and about 100 mM NaCl. In some instances, the histidine/sucrose/NaCl buffer comprises from about 5 mM histidine to about 50 mM histidine, from about 50 mM sucrose to about 300 mM sucrose, and from about 5 mM NaCl to about 200 mM NaCl. In some cases, the histidine/sucrose/NaCl buffer comprises about 10 mM histidine, about 100 mM sucrose, and about 100 mM NaCl. In some instances, the citrate/sucrose/NaCl buffer comprises from about 5 mM citrate to about 50 mM citrate, from about 50 mM sucrose to about 300 mM sucrose, and from about 5 mM NaCl to about 200 mM NaCl. In some cases, the citrate/sucrose/NaCl buffer comprises about 10 mM citrate, about 100 mM sucrose, and about 100 mM NaCl. In some instances, the acetate/sucrose/NaCl buffer comprises from about 5 mM acetate to about 50 mM acetate, from about 50 mM sucrose to about 300 mM sucrose, and from about 5 mM NaCl to about 200 mM NaCl. In some cases, the acetate/sucrose/NaCl buffer comprises about 10 mM acetate, about 100 mM sucrose, and about 100 mM NaCl. In some cases, the L-Wnt undergoes a sterile filtration step.

In some instances, the L-Wnt is stored under nitrogen. In some instances, the L-Wnt is stable under nitrogen without substantial loss of activity.

In some instances, the L-Wnt is stored at a temperature of between about 1° C. and about 8° C. In some instances, the L-Wnt is stable at a temperature of at least about 1° C., 2° C., 3° C., 4° C., 5° C., 6° C., 7° C., 8° C., or more without substantial loss of activity. In some instances, the L-Wnt is stable at a temperature of at most about 1° C., 2° C., 3° C., 4° C., 5° C., 6° C., 7° C., 8° C., or less without substantial loss of activity.

In some instances, the L-Wnt is stored at a temperature of from about −80° C. to about −20° C. In some instances, the L-Wnt is stable at a temperature of about −80° C. without substantial loss of activity. In some instances, the L-Wnt is stable at a temperature of about −20° C. without substantial loss of activity.

In some embodiments, the L-Wnt is stable for at least about 10, 15, 20, 25, 30, 35, 40, 45, 50, 55, 60, 65, 70, 75, 80, 85, 90, 95, 100, 101, 102, 103, 104, 105, 106, 107, 108, 109, 110, 115, 120, 130, 140, 150, 160, 170, 180, 190, 200, 300, 356, 400, 700, 1000 days, or more without substantial loss of activity. In some embodiments, the L-Wnt is stable for at most about 10, 15, 20, 25, 30, 35, 40, 45, 50, 55, 60, 65, 70, 75, 80, 85, 90, 95, 100, 101, 102, 103, 104, 105, 106, 107, 108, 109, 110, 115, 120, 130, 140, 150, 160, 170, 180, 190, 200, 300, 356, 400, 700, 1000 days, or less without substantial loss of activity.

In some embodiments, the L-Wnt3A undergoes a centrifugation step and is then suspended in a buffer. Exemplary buffers include, but are not limited to, phosphate buffered saline (PBS) or a sucrose-based buffer such as a phosphate/sucrose buffer, a histidine/sucrose buffer, a citrate/sucrose buffer, an acetate/sucrose buffer, a sucrose/NaCl based buffer, a phosphate/sucrose/NaCl buffer, a histidine/sucrose/NaCl buffer, a citrate/sucrose/NaCl buffer, or an acetate/sucrose/NaCl buffer. In some instances, the sucrose-based buffer comprises from about 50 mM sucrose to about 500 mM sucrose. In some cases, the sucrose-based buffer comprises about 300 mM sucrose. In some instances, the phosphate/sucrose buffer comprises from about 5 mM phosphate to about 50 mM phosphate and from about 50 mM sucrose to about 500 mM sucrose. In some cases, the phosphate/sucrose buffer comprises about 10 mM phosphate and about 300 mM sucrose. In some cases, the histidine/sucrose buffer comprises about 10 mM histidine and about 300 mM sucrose. In some instances, the citrate/sucrose buffer comprises from about 5 mM citrate to about 50 mM citrate and from about 50 mM sucrose to about 500 mM sucrose. In some cases, the citrate/sucrose buffer comprises about 10 mM citrate and about 300 mM sucrose. In some instances, the acetate/sucrose buffer comprises from about 5 mM acetate to about 50 mM acetate and from about 50 mM sucrose to about 500 mM sucrose. In some cases, the acetate/sucrose buffer comprises about 10 mM acetate and about 300 mM sucrose. In some instances, the sucrose/NaCl-based buffer comprises from about 50 mM sucrose to about 300 mM sucrose and from about 5 mM NaCl to about 200 mM NaCl. In some cases, the sucrose/NaCl-based buffer comprises about 100 mM sucrose and 100 mM NaCl. In some instances, the phosphate/sucrose/NaCl buffer comprises from about 5 mM phosphate to about 50 mM phosphate, from about 50 mM sucrose to about 300 mM sucrose, and from about 5 mM NaCl to about 200 mM NaCl. In some cases, the phosphate/sucrose/NaCl buffer comprises about 10 mM phosphate, about 100 mM sucrose, and about 100 mM NaCl. In some instances, the histidine/sucrose/NaCl buffer comprises from about 5 mM histidine to about 50 mM histidine, from about 50 mM sucrose to about 300 mM sucrose, and from about 5 mM NaCl to about 200 mM NaCl. In some cases, the histidine/sucrose/NaCl buffer comprises about 10 mM histidine, about 100 mM sucrose, and about 100 mM NaCl. In some instances, the citrate/sucrose/NaCl buffer comprises from about 5 mM citrate to about 50 mM citrate, from about 50 mM sucrose to about 300 mM sucrose, and from about 5 mM NaCl to about 200 mM NaCl. In some cases, the citrate/sucrose/NaCl buffer comprises about 10 mM citrate, about 100 mM sucrose, and about 100 mM NaCl. In some instances, the acetate/sucrose/NaCl buffer comprises from about 5 mM acetate to about 50 mM acetate, from about 50 mM sucrose to about 300 mM sucrose, and from about 5 mM NaCl to about 200 mM NaCl. In some cases, the acetate/sucrose/NaCl buffer comprises about 10 mM acetate, about 100 mM sucrose, and about 100 mM NaCl.

In some embodiments, the L-Wnt3A undergoes a filtration step. In some instances, the filtration step comprises an ultrafiltration, a diafiltration, nanofiltration, steril filtration, or a combination thereof. Exemplary filtration membranes include, but are not limited to, cellulose acetate (CA), polysulfone (PS), polyether sulfone (PES), polyacrilonitrile (PAN), polyvinylidiene fluoride (PVDF), polypropylene (PP), polyethylene (PE), and polyvinyl chloride (PVC). In some instances, the L-Wnt3A undergoes one or more filtrations such as an ultrafiltration, a diafiltration, nanofiltration, a steril filtration, or a combination thereof. In some instances, L-Wnt3A undergoes an ultrafiltration and a nanofiltration for removal of one or more biological contaminant such as protein contaminants and microbial contaminants. In some instances, the nanofiltration removes one or more viral contaminants. In some instances, the L-Wnt3A further undergoes a diafiltration step for buffer exchange. Exemplary buffers include, but are not limited to, phosphate buffered saline (PBS) or a sucrose-based buffer such as a phosphate/sucrose buffer, a histidine/sucrose buffer, a citrate/sucrose buffer, an acetate/sucrose buffer, a sucrose/NaCl based buffer, a phosphate/sucrose/NaCl buffer, a histidine/sucrose/NaCl buffer, a citrate/sucrose/NaCl buffer, or an acetate/sucrose/NaCl buffer. In some instances, the sucrose-based buffer comprises from about 50 mM sucrose to about 500 mM sucrose. In some cases, the sucrose-based buffer comprises about 300 mM sucrose. In some instances, the phosphate/sucrose buffer comprises from about 5 mM phosphate to about 50 mM phosphate and from about 50 mM sucrose to about 500 mM sucrose. In some cases, the phosphate/sucrose buffer comprises about 10 mM phosphate and about 300 mM sucrose. In some cases, the histidine/sucrose buffer comprises about 10 mM histidine and about 300 mM sucrose. In some instances, the citrate/sucrose buffer comprises from about 5 mM citrate to about 50 mM citrate and from about 50 mM sucrose to about 500 mM sucrose. In some cases, the citrate/sucrose buffer comprises about 10 mM citrate and about 300 mM sucrose. In some instances, the acetate/sucrose buffer comprises from about 5 mM acetate to about 50 mM acetate and from about 50 mM sucrose to about 500 mM sucrose. In some cases, the acetate/sucrose buffer comprises about 10 mM acetate and about 300 mM sucrose. In some instances, the sucrose/NaCl-based buffer comprises from about 50 mM sucrose to about 300 mM sucrose and from about 5 mM NaCl to about 200 mM NaCl. In some cases, the sucrose/NaCl-based buffer comprises about 100 mM sucrose and 100 mM NaCl. In some instances, the phosphate/sucrose/NaCl buffer comprises from about 5 mM phosphate to about 50 mM phosphate, from about 50 mM sucrose to about 300 mM sucrose, and from about 5 mM NaCl to about 200 mM NaCl. In some cases, the phosphate/sucrose/NaCl buffer comprises about 10 mM phosphate, about 100 mM sucrose, and about 100 mM NaCl. In some instances, the histidine/sucrose/NaCl buffer comprises from about 5 mM histidine to about 50 mM histidine, from about 50 mM sucrose to about 300 mM sucrose, and from about 5 mM NaCl to about 200 mM NaCl. In some cases, the histidine/sucrose/NaCl buffer comprises about 10 mM histidine, about 100 mM sucrose, and about 100 mM NaCl. In some instances, the citrate/sucrose/NaCl buffer comprises from about 5 mM citrate to about 50 mM citrate, from about 50 mM sucrose to about 300 mM sucrose, and from about 5 mM NaCl to about 200 mM NaCl. In some cases, the citrate/sucrose/NaCl buffer comprises about 10 mM citrate, about 100 mM sucrose, and about 100 mM NaCl. In some instances, the acetate/sucrose/NaCl buffer comprises from about 5 mM acetate to about 50 mM acetate, from about 50 mM sucrose to about 300 mM sucrose, and from about 5 mM NaCl to about 200 mM NaCl. In some cases, the acetate/sucrose/NaCl buffer comprises about 10 mM acetate, about 100 mM sucrose, and about 100 mM NaCl. In some cases, the L-Wnt3A undergoes a sterile filtration step.

In some instances, the L-Wnt3A is stored under nitrogen. In some instances, the L-Wnt3A is stable under nitrogen without substantial loss of activity.

In some instances, the L-Wnt3A is stored at a temperature of between about 1° C. and about 8° C. In some instances, the L-Wnt3A is stable at a temperature of at least about 1° C., 2° C., 3° C., 4° C., 5° C., 6° C., 7° C., 8° C., or more without substantial loss of activity. In some instances, the L-Wnt3A is stable at a temperature of at most about 1° C., 2° C., 3° C., 4° C., 5° C., 6° C., 7° C., 8° C., or less without substantial loss of activity.

In some instances, the L-Wnt3A is stored at a temperature of from about −80° C. to about −20° C. In some instances, the L-Wnt3A is stable at a temperature of about −80° C. without substantial loss of activity. In some instances, the L-Wnt3A is stable at a temperature of about −20° C. without substantial loss of activity.

In some embodiments, the L-Wnt3A is stable for at least about 10, 20, 30, 40, 50, 60, 70, 80 90, 100, 110, 120, 130, 140, 150, 160, 170, 180, 190, 200, 300, 356, 400, 700, 1000 days, or more without substantial loss of activity. In some embodiments, the L-Wnt3A is stable for at most about 10, 20, 30, 40, 50, 60, 70, 80 90, 100, 110, 120, 130, 140, 150, 160, 170, 180, 190, 200, 300, 356, 400, 700, 1000 days, or less without substantial loss of activity.

In some instances, the term “without substantial loss of activity” refers to the functional activity of a liposomal Wnt polypeptide is near to that of the corresponding native Wnt polypeptide in the absence of a liposome. In some instances, the functional activity of the liposomal Wnt polypeptide is at least about 100%, 99%, 95%, 90%, 85%, 80%, 75%, 70%, 60%, 50%, 40%, or more compared to the functional activity of the native Wnt polypeptide. In some instances, the functional activity of the liposomal Wnt polypeptide is at most about 100%, 99%, 95%, 90%, 85%, 80%, 75%, 70%, 60%, 50%, 40%, or less compared to the functional activity of the native Wnt polypeptide. In some instances, the functional activity of the Wnt polypeptides is detected using assays such as for example mass spectroscopy, assays associated with biomarker analysis which are described elsewhere herein, transplant surgery such as sub-renal capsule transplant surgery, spinal fusion surgery, ALP, TRAP, and TUNEL staining, immunohistochemistry, and Micro-CT analyses and quantification of graft growth.

In some instances, the term “stable” refers to Wnt polypeptides as in a folded state and is not unfolded or degraded. In some instances, the term “stable” also refers to Wnt polypeptides retaining functional activity without substantial loss of activity. In some instances, assays used to determine stability assays that establish the activity of the Wnt polypeptides, as such those described above, and also include such as LSL cell-based assays such as mice LSL cell-based assay.

In some embodiments, the quantity, purity, potency, and safety of the Wnt polypeptide and liposomal Wnt polypeptide (e.g., Wnt3A polypeptide and L-Wnt3A, respective) are further evaluated. In some instances, the quantity (or concentration) of the Wnt polypeptide and liposomal Wnt polypeptide (e.g., Wnt3A polypeptide and L-Wnt3A, respective) is determined by utilizing a chromatographic method (e.g., a HPLC method). In some instances, the HPLC method is a RP-HPLC method.

In some instances, the purity of the Wnt polypeptide and liposomal Wnt polypeptide (e.g., Wnt3A polypeptide and L-Wnt3A, respective) is determined by utilizing a chromatographic method (e.g., a HPLC method), size separation method (e.g., SDS-PAGE), or a charge separation method (a capillary isoelectric focusing (cIEF) method).

In some embodiments, the potency of the Wnt polypeptide and liposomal Wnt polypeptide (e.g., Wnt3A polypeptide and L-Wnt3A, respective) is determined by utilizing the LSL assay described herein.

In some embodiments, the safety of the Wnt polypeptide and liposomal Wnt polypeptide (e.g., Wnt3A polypeptide and L-Wnt3A, respective) is determined by utilizing, e.g., a microbial enumeration test (e.g., as described in USP 61 (USP29-NF24)) and/or an endotoxin test (e.g., as decribed in USP 85 (USP29-NF24)).

In some embodiments, the osmolality of the Wnt polypeptide and liposomal Wnt polypeptide (e.g., Wnt3A polypeptide and L-Wnt3A, respective) is determined. In some instances, the osmolality of the Wnt polypeptide and liposomal Wnt polypeptide (e.g., Wnt3A polypeptide and L-Wnt3A, respective) is determined according to the guideline as described in USP 785 (USP29-NF24).

In some embodiments, the Wnt polypeptide and liposomal Wnt polypeptide (e.g., Wnt3A polypeptide and L-Wnt3A, respective) comprises less than

### Expression Constructs

In some embodiments, a Wnt polypeptide comprising one or more variants is produced by recombinant methods. In some instances, the Wnt polypeptide is a Wnt3A, Wnt5A, or a Wnt10B polypeptide. In some instances, the Wnt polypeptide comprising one or more variants is a Wnt3A polypeptide. In some instances, the Wnt polypeptide comprising one or more variants is a Wnt5A polypeptide. In some instances, the Wnt polypeptide comprising one or more variants is a Wnt10B polypeptide.

Amino acid sequence variants, including variants that are truncated at the C-terminus, are prepared by introducing appropriate nucleotide changes into the Wnt polypeptide DNA. Such variants represent insertions, substitutions, and/or specified deletions of, residues within or at one or both of the ends of the amino acid sequence of a naturally occurring Wnt polypeptide. Any combination of insertion, substitution, and/or specified deletion, e.g. truncation, is made to arrive at the final construct, provided that the final construct possesses the desired biological activity as defined herein. The amino acid changes also may alter post-translational processes of the Wnt polypeptide, such as changing the number or position of glycosylation sites, altering the membrane anchoring characteristics, and/or altering the intracellular location of the Wnt polypeptide by inserting, deleting, or otherwise affecting the leader sequence of the Wnt polypeptide.

In some embodiments, the one or more variants within a Wnt polypeptide comprise a substitution, insertion, deletion, or a combination thereof. In some instances, the Wnt3A polypeptide comprises a substitution, insertion, deletion, or a combination thereof. In some cases, the Wnt5A polypeptide comprises a substitution, insertion, deletion, or a combination thereof. In other cases, the Wnt10B polypeptide comprises a substitution, insertion, deletion, or a combination thereof.

In some cases, the DNA encoding a Wnt3A polypeptide is represented by SEQ ID NO: 1 or SEQ ID NO: 2. In some cases, the DNA encoding a Wnt3A polypeptide is prepared, e.g. by truncating a sequence of SEQ ID NO: 1, or by utilizing the sequence of SEQ ID NO: 2. In some instances, the Wnt polypeptide-encoding gene is also obtained by oligonucleotide synthesis, amplification, etc. as known in the art.

The nucleic acid (e.g., cDNA or genomic DNA) encoding the Wnt polypeptide is inserted into a replicable vector for expression. Many such vectors are available. The vector components generally include, but are not limited to, one or more of the following: an origin of replication, one or more marker genes, an enhancer element, a promoter, and a transcription termination sequence. Preferably a GMP compatible vector is selected, for example the commercially available vectors OpticVec, pTarget, pcDNA4TO4, pcDNA4.0, and the like.

In some instances, the vector comprising a first nucleic acid encoding a Wnt polypeptide further comprises a second nucleic acid encoding a chaperone, operably linked to the first nucleic acid. In some instances, the chaperone is a Frizzled protein, Wntless, Afamin, or Porcupine. In some cases, the chaperone is Frizzled-8. In some cases, the chaperone is Frizzled-8 fusion protein (e.g., SEQ ID NO: 5 or SEQ ID NO: 18). In some instances, the vector is a multicistronic (e.g., a bicistronic) vector in which the first nucleic acid and the second nucleic acid are under the same promoter and the vector region between the first nucleic acid and the second nucleic acid comprises an IRES element or a 2A peptide. In some instances, the 2A peptide comprises: T2A ([GSG]-EGRGSLLTCGDVEENPGP) (SEQ ID NO: 30), P2A ([GSG-]-ATNFSLLKQAGDVEENPGP) (SEQ ID NO: 31), E2A ([GSG]-QCTNYALLKLAGDVESNPGP) (SEQ ID NO: 32), and F2A ([GSG]-VKQTLNFDLLKLAGDVESNPGP) (SEQ ID NO: 33), In some instances, the vector comprises the first nucleic acid and the second nucleic acid but the two nucleic acids are under two different promoters.

In some embodiments, the first nucleic acid encoding a Wnt polypeptide and the second nucleic acid encoding a chaperone are constructed in two different vectors.

In some embodiments, an expression vector that is tolerant of a minimal serum culture condition is used. In some instances, the minimal serum culture condition includes reduced-serum culture condition, protein-free culture condition, chemically defined media culture condition, or serum-free culture condition. In some embodiments, an expression vector that is tolerant of a reduced-serum culture condition is used. In some embodiments, an expression vector that is tolerant of a protein-free culture condition is used. In some embodiments, an expression vector that is tolerant of a chemically defined media culture condition is used.

In some embodiments, an expression vector that is tolerant of a serum-free medium condition is used. In some cases, the expression vector leads to a high copy number of the desired transcript and secretion of the protein of interest. In some instances, the expression vector is compatible with cGMP compatible mammalian cell lines. Non-limiting examples of mammalian expression vectors include pOptivec vector, pTargeT™ vector, BacMam pCMV-Dest vector, Flp-In™ core system, Gateway® suite of vectors, HaloTag® vector, Flexi® vector, pCMVTNT™ vector, pcDNA4.0, and pcDNA™4/TO vector. In some embodiments, the expression vector is selected from pOptivec and pTargeT™ vectors. The pOptivec vector is a TOPO® adapted bicistronic plasmid which allows rapid cloning of a gene containing a mammalian secretion signal and the gene of interest downstream of the CMV promoter. The dihydrofolate reductase selection markers allows for rapid selection. In some cases, this vector is used for transient transfection of CHO-S cells. In some instances, the pTargeT™ vector is used for transient transfection of CHO-S cells and for creating a stable cell line expressing a Wnt polypeptide (e.g. Wnt3A).

The coding sequence will also include a signal sequence that allows secretion of the Wnt. The signal sequence may be a component of the vector, or it may be a part of the Wnt encoding DNA that is inserted into the vector. A heterologous signal sequence selected preferably is one that is recognized and processed (i.e., cleaved by a signal peptidase) by the host cell. In mammalian cell expression the native signal sequence may be used, or other mammalian signal sequences may be suitable, such as signal sequences from other animal Wnt polypeptide, and signal sequences from secreted polypeptides of the same or related species, as well as viral secretory leaders, for example, the herpes simplex gD signal.

Expression vectors may contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors will contain a promoter that is recognized by the host organism and is operably linked to the Wnt coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known. Both a native Wnt polypeptide promoter sequence and many heterologous promoters may be used to direct expression of a Wnt polypeptide. However, heterologous promoters are preferred, as they generally permit greater transcription and higher yields.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment.

Transcription may be increased by inserting an enhancer sequence into the vector. Enhancers are cis-acting elements of DNA, usually about from 10 to 300 bp, which act on a promoter to increase its transcription Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from an eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors used in mammalian host cells will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding Wnt polypeptide.

Construction of suitable vectors containing one or more of the above-listed components employs standard techniques. Isolated plasmids or DNA fragments are cleaved, tailored, and re-ligated in the form desired to generate the vectors required.

In some instances, expression vectors that provide for the transient expression in mammalian cells are used. In general, transient expression involves the use of an expression vector that is able to replicate efficiently in a host cell, such that the host cell accumulates many copies of the expression vector and, in turn, synthesizes high levels of a desired polypeptide encoded by the expression vector. Transient expression systems, comprising a suitable expression vector and a host cell, allow for the convenient positive identification of polypeptides encoded by cloned DNAs, as well as for the rapid screening of such polypeptides for desired biological or physiological properties.

In some embodiments, expression vector that provide for stable expression in mammalian cells are used. In such cases, the stable expression system, comprising a suitable expression vector and a host cell, provides for a large scale production (e.g., more than 40 L, more than 50 L, more than 100 L, more than 150 L, more than 200 L, more than 250 L, or more than 300 L culture).

In some instances, serum-free media is used. Non-limiting examples of serum-free media include CD CHO medium, CD CHO AGT™ medium, CD OptiCHO™ medium, CHO-S-SFM II (optionally including hypoxanthine and thymidine), CD 293 AGT™ medium, Adenovirus Expression Medium (AEM), FreeStyle™ 293 Expression medium, FreeStyle™ CHO Expression medium, CD FortiCHO™ medium, EX-CELL® 302 Serum-Free medium, EX-CELL® 325 PF CHO Serum-Free medium, EX-CELL® CD CHO-2 medium animal-component free, EX-CELL® CD CHO-3 medium, EX-CELL® CDHO DHFR⁻ medium animal-component free, and ActiPro medium.

The methods of the present invention may be performed so as to conform with FDA or WHO guidelines for GMP production. Guidelines for such may be obtained from the relevant regulatory agency. See, for example, “WHO good manufacturing practices: main principles for pharmaceutical products. Annex 3 in: WHO Expert Committee on Specifications for Pharmaceutical Preparations. Forty-fifth report. Geneva, World Health Organization, 2011 (WHO Technical Report Series, No. 961)”; “ICH Q5B guideline. Analysis of the expression construct in cells used for production of r-DNA derived protein products. Geneva, International Conference on Harmonisation of Technical Requirements for Registration of Pharmaceuticals for Human Use, 1995”; “Handbook: good laboratory practice (GLP): quality practices for regulated non-clinical research and development, 2nd ed. Geneva, UNDP/World Bank/WHO, Special Programme for Research and Training in Tropical Diseases, 2009”; each herein specifically incorporated by reference.

Typically, recombinant DNA-derived biotherapeutics are produced using a cell bank system which involves a manufacturer's working cell bank (WCB) derived from a master cell bank. The present invention includes frozen aliquots of Chinese Hamster Ovary (CHO) (e.g., CHO-S or CHO-K1) cells transfected with a vector for secretion of the WNT3A protein, which cells can be used as a master cell bank or as a working cell bank.

In some embodiments, the production scale (or the cell culture scale) is more than 40 L, more than 50 L, more than 100 L, more than 150 L, more than 200 L, more than 250 L, or more than 300 L. In some instances, the production scale (or the cell culture scale) is more than 100 L. In some instances, the production scale (or the cell culture scale) is more than 200 L. In some instances, the production scale (or the cell culture scale) is more than 300 L. In some instances, the production scale (or the cell culture scale) is about 100 L. In some instances, the production scale (or the cell culture scale) is about 200 L. In some instances, the production scale (or the cell culture scale) is about 300 L.

In some embodiments, the host cells are grown in a suspension.

### Cell Lines

In some embodiments, a cGMP compatible cell line is transfected with an expression vector encoding a Wnt polypeptide. Exemplary cGMP compatible cell line includes mammalian cell lines such as Chinese Hamster Ovary (CHO) cell line, human embryonic kidney (HEK) cell line, or baby hamster kidney (BHK) cell line; or insect cell lines such as Sf9 cell line, Sf21 cell line, Tn-368 cell line, or High Five (BTI-TN-5B1-4) cell line.

In some instances, an expression vector encoding a Wnt polypeptide is transfected in a cGMP compatible cell line selected from Chinese Hamster Ovary (CHO) cell line, human embryonic kidney (HEK) cell line, baby hamster kidney (BHK) cell line, Sf9 cell line, Sf21 cell line, Tn-368 cell line, or High Five (BTI-TN-5B1-4) cell line. In some instances, an expression vector encoding a Wnt polypeptide is transfected in a CHO cell line. In some instances, an expression vector encoding a Wnt polypeptide is transfected in a BHK cell line. In some instances, an expression vector encoding a Wnt polypeptide is transfected in a HEK cell line. In some instances, an expression vector encoding a Wnt polypeptide is transfected in a Sf9 cell line. In some instances, an expression vector encoding a Wnt polypeptide is transfected in a Sf21 cell line. In some instances, an expression vector encoding a Wnt polypeptide is transfected in a Tn-368 cell line. In some instances, an expression vector encoding a Wnt polypeptide is transfected in a High Five cell line. In some cases, the Wnt polypeptide is Wnt3A polypeptide, Wnt 5A polypeptide, or Wnt 10B polypeptide.

In some embodiments, the Wnt polypeptide is Wnt3A polypeptide. In some instances, an expression vector encoding Wnt3A polypeptide is transfected in a cGMP compatible cell line selected from Chinese Hamster Ovary (CHO) cell line, human embryonic kidney (HEK) cell line, baby hamster kidney (BHK) cell line, Sf9 cell line, Sf21 cell line, Tn-368 cell line, or High Five (BTI-TN-5B1-4) cell line. In some instances, an expression vector encoding Wnt3A polypeptide is transfected in a CHO cell line. In some instances, an expression vector encoding Wnt3A polypeptide is transfected in a BHK cell line. In some instances, an expression vector encoding Wnt3A polypeptide is transfected in a HEK cell line. In some instances, an expression vector encoding Wnt3A polypeptide is transfected in a Sf9 cell line. In some instances, an expression vector encoding Wnt3A polypeptide is transfected in a Sf21 cell line. In some instances, an expression vector encoding Wnt3A polypeptide is transfected in a Tn-368 cell line. In some instances, an expression vector encoding Wnt3A polypeptide is transfected in a High Five cell line.

Exemplary CHO cell lines include, but are not limited to, CHO-S, CHO-K1, CHO-DXB11 (or CHO-DUKX), and CHO-DG44 cell lines. In some instances, an expression vector encoding a Wnt polypeptide is transfected in a CHO-S cell line or a CHO-K1 cell line. In some cases, the Wnt polypeptide is Wnt3A polypeptide, Wnt5A polypeptide, or Wnt10B polypeptide. In some instances, an expression vector encoding Wnt3A polypeptide is transfected in a CHO-S cell line. In some instances, an expression vector encoding Wnt3A polypeptide is transfected in a CHO-K1 cell line. In some cases, an expression vector encoding SEQ ID NO: 1 or SEQ ID NO: 2 of Wnt3A polypeptide is transfected in a CHO-S cell line. In some cases, an expression vector encoding SEQ ID NO: 1 or SEQ ID NO: 2 of Wnt3A polypeptide is transfected in a CHO-K1 cell line. In additional cases, an expression vector encoding a Wnt3A polypeptide comprising a variant (e.g., a deletion or truncation) is transfected in a CHO-S cell line. In additional cases, an expression vector encoding a Wnt3A polypeptide comprising a variant (e.g., a deletion or truncation) is transfected in a CHO-K1 cell line.

In some instances, the combination of CHO-S cells transfected with an expression vector encoding Wnt3A polypeptide comprising a deletion or a truncation allows effective secretion of the protein into minimal serum culture medium (e.g., serum-free condition). In some cases, the deletion or truncation is a C-terminus deletion or truncation. In some instances, the Wnt3A polypeptide is as illustrated in SEQ ID NO: 1. In some cases, the combination of CHO-S cells transfected with an expression vector encoding Wnt3A polypeptide in which, relative to SEQ ID NO:1 (BC103921), the C-terminus is truncated, allows effective secretion of the protein into culture medium in the absence of serum or other animal products.

In some instances, the combination of CHO-K1 cells transfected with an expression vector encoding Wnt3A polypeptide comprising a deletion or a truncation allows effective secretion of the protein into minimal serum culture medium (e.g., serum-free condition). In some cases, the deletion or truncation is a C-terminus deletion or truncation. In some instances, the Wnt3A polypeptide is as illustrated in SEQ ID NO: 1. In some cases, the combination of CHO-S cells transfected with an expression vector encoding Wnt3A polypeptide in which, relative to SEQ ID NO:1 (BC103921), the C-terminus is truncated, allows effective secretion of the protein into culture medium in the absence of serum or other animal products. In some cases, the CHO-K1 cells are grown as a suspension.

As described elsewhere herein, the minimal serum medium sometimes comprises less than 9% serum. In some cases, the serum is FBS. In some cases, the FBS presents in the minimal serum medium is at most about 0.05%, 0.1%, 0.5%, 1%, 2%, 3%, 4%, 5%, 6%, 7%, 8%, 9%, or less. In some cases, the FBS presents in the minimal serum medium is at least about 0.05%, 0.1%, 0.5%, 1%, 2%, 3%, 4%, 5%, 6%, 7%, 8%, 9%, or more. In some cases, the FBS presents in the minimal serum medium is about 0.05%. In some cases, the FBS presents in the minimal serum medium is about 0.1%. In some cases, the FBS presents in the minimal serum medium is about 0.5%. In some cases, the FBS presents in the minimal serum medium is about 1%. In some cases, the FBS presents in the minimal serum medium is about 2%. In some cases, the FBS presents in the minimal serum medium is about 3%. In some cases, the FBS presents in the minimal serum medium is about 4%. In some cases, the FBS presents in the minimal serum medium is about 5%. In some cases, the FBS presents in the minimal serum medium is about 6%. In some cases, the FBS presents in the minimal serum medium is about 7%. In some cases, the FBS presents in the minimal serum medium is about 8%. In some cases, the FBS presents in the minimal serum medium is about 9%. In other cases, the minimal serum medium is a serum-free medium.

Sometimes, the minimal serum medium comprises components such as peptides and/or polypeptides obtained from plant hydrolysates but not proteins or components of animal origin. In other cases, the minimal serum medium comprises recombinant proteins and/or hormones and does not comprise FBS, bovine serum albumin, or human serum albumin. In additional cases, the minimal serum medium comprises low molecular weight constituents and optionally synthetic peptides and/or hormones.

In some embodiments, the minimal serum medium contains one or more additional supplement. In some embodiments, the additional supplement is a lipid supplement. Non-limiting examples of lipid supplement include Lipid Mixture 1 (Sigma-Aldrich), Lipid Mixture 2 (Sigma-Aldrich), Lipogro® (Rocky Mountain Biologicals), and Chemically Defined Lipid Concentration (Life Technologies). In some embodiments, the serum-free medium contains a lipid supplement.

In some instances, the minimal serum medium is a serum free, chemically defined medium. In some cases, the serum free, chemically defined medium is substantially free of animal-derived components.

In some embodiments, the methods of the disclosure comprise culturing in serum-free medium CHO cells (e.g., CHO-S cells or CHO-K1 cells) transfected with an expression vector comprising a Wnt polypeptide (e.g., Wnt3A polypeptide) comprising a signal sequence for secretion, which can be the native Wnt (e.g., Wnt3A) signal sequence or a heterologous signal sequence, operably linked to a promoter, under conditions in which the Wnt polypeptide (e.g., Wnt3A polypeptide) is expressed and secreted. In some instances, the Wnt polypeptide is a C-terminal truncated Wnt polypeptide (e.g., Wnt3A polypeptide) comprising a signal sequence for secretion, which can be the native Wnt (e.g., Wnt3A) signal sequence or a heterologous signal sequence, operably linked to a promoter, under conditions in which the Wnt polypeptide (e.g., Wnt3A polypeptide) is expressed and secreted. In some embodiments, the methods further comprise an initial step of transfecting the cells with the expression vector. In some embodiment the methods comprise purifying the polypeptide thus produced from the medium. In some embodiments the Wnt polypeptide (e.g., Wnt3A polypeptide) is purified to a degree suitable for GMP clinical use. In some embodiments the Wnt polypeptide (e.g., Wnt3A polypeptide) thus purified is packaged in a unit dose formulation.

In some embodiments, the CHO cells are grown in suspension.

In other embodiments, the CHO cells are adherent.

In some embodiments, the medium comprises a serum substitute. In some embodiments the serum substitute is free of animal products. In some embodiments the serum substitute comprises purified proteins, e.g. one or more of insulin, transferrin, bovine serum albumin, human serum albumin, etc., but which lacks, for example, growth factors, steroid hormones, glucocorticoids, cell adhesion factors, detectable Ig, mitogens, etc. The serum substitute may be present at a concentration in the medium of up to about 0.1%, up to about 0.25%, up to about 0.5%, up to about 0.75%, up to about 1%, up to about 2.5%, up to about 5%, up to about 7.5%, or up to about 10%. The serum substitute may be present at a concentration in the medium of up to about 0.1%. The serum substitute may be present at a concentration in the medium of up to about 0.25%. The serum substitute may be present at a concentration in the medium of up to about 0.5%. The serum substitute may be present at a concentration in the medium of up to about 0.75%. The serum substitute may be present at a concentration in the medium of up to about 1%. The serum substitute may be present at a concentration in the medium of up to about 2.5%. The serum substitute may be present at a concentration in the medium of up to about 5%. The serum substitute may be present at a concentration in the medium of up to about 7.5%. The serum substitute may be present at a concentration in the medium of up to about 10%.

Suitable medium may be selected from those known in the art, including without limitation DMEM, RPMI-1640, MEM, Iscove's, CHO Cell Medium; and the like. Suitable serum substitutes include those produced with no animal products, or those with only purified animal protein components. Commercially available supplements suitable for this purpose include, without limitation, CellEss, ITS (e.g., ITS3 or ITS3+), Excyte, OneShot, Knockout, and the like as known in the art. In some instances, the ITS supplement is a supplement comprising a mixture of insulin, transferrin, and selenium. The medium may further comprise, without limitation, such components as GlutaMax™ (a glutamine-based dipeptide), antibiotic (e.g. doxycycline), G418, non-essential amino acids, blasticidine, etc.

The level of secretion of the Wnt polypeptide into the serum-free culture medium may be at least about 10 ng/ml, at least about 25 ng/ml, at least about 50 ng/ml, at least about 75 ng/ml, at least about 100 ng/ml, at least about 250 ng/ml, at least about 500 ng/ml, at least about 750 ng/ml, at least about 1 μg/ml, at least about 1.1 μg/ml, at least about 1.25 μg/ml, at least about 1.5 μg/ml, at least about 1.75 μg/ml, at least about 2.5 μg/ml, at least about 5 μg/ml, at least about 7.5 μg/ml, at least about 10 μg/ml, at least about 15 μg/ml, at least about 20 μg/ml, at least about 25 μg/ml, at least about 30 μg/ml, or more. The level of secretion of the Wnt polypeptide into the serum-free culture medium may be at least about 10 ng/ml. The level of secretion of the Wnt polypeptide into the serum-free culture medium may be at least about 25 ng/ml. The level of secretion of the Wnt polypeptide into the serum-free culture medium may be at least about 50 ng/ml. The level of secretion of the Wnt polypeptide into the serum-free culture medium may be at least about 75 ng/ml. The level of secretion of the Wnt polypeptide into the serum-free culture medium may be at least about 100 ng/ml. The level of secretion of the Wnt polypeptide into the serum-free culture medium may be at least about 250 ng/ml. The level of secretion of the Wnt polypeptide into the serum-free culture medium may be at least about 500 ng/ml. The level of secretion of the Wnt polypeptide into the serum-free culture medium may be at least about 750 ng/ml. The level of secretion of the Wnt polypeptide into the serum-free culture medium may be at least about 1 μg/ml. The level of secretion of the Wnt polypeptide into the serum-free culture medium may be at least about 1.1 μg/ml. The level of secretion of the Wnt polypeptide into the serum-free culture medium may be at least about 1.25 μg/ml. The level of secretion of the Wnt polypeptide into the serum-free culture medium may be at least about 1.5 μg/ml. The level of secretion of the Wnt polypeptide into the serum-free culture medium may be at least about 1.75 μg/ml. The level of secretion of the Wnt polypeptide into the serum-free culture medium may be at least about 2.5 μg/ml. The level of secretion of the Wnt polypeptide into the serum-free culture medium may be at least about 5 μg/ml. The level of secretion of the Wnt polypeptide into the serum-free culture medium may be at least about 7.5 μg/ml. The level of secretion of the Wnt polypeptide into the serum-free culture medium may be at least about 10 μg/ml. The level of secretion of the Wnt polypeptide into the serum-free culture medium may be at least about 15 μg/ml. The level of secretion of the Wnt polypeptide into the serum-free culture medium may be at least about 20 μg/ml. The level of secretion of the Wnt polypeptide into the serum-free culture medium may be at least about 25 μg/ml. The level of secretion of the Wnt polypeptide into the serum-free culture medium may be at least about 30 μg/ml. In some instances, the Wnt polypeptide is Wnt3A polypeptide. In some cases, the Wnt polypeptide is Wnt5A polypeptide. In some cases, the Wnt polypeptide is Wnt 10B polypeptide.

In some instances, the Wnt polypeptide is Wnt3A polypeptide. In some cases, the level of secretion of the Wnt3A polypeptide into the serum-free culture medium is at least about 10 ng/ml, at least about 25 ng/ml, at least about 50 ng/ml, at least about 75 ng/ml, at least about 100 ng/ml, at least about 250 ng/ml, at least about 500 ng/ml, at least about 750 ng/ml, at least about 1 μg/ml, at least about 1.1 μg/ml, at least about 1.25 μg/ml, at least about 1.5 μg/ml, at least about 1.75 μg/ml, at least about 2.5 μg/ml, at least about 5 μg/ml, at least about 7.5 μg/ml, at least about 10 μg/ml, at least about 15 μg/ml, at least about 20 μg/ml, at least about 25 μg/ml, at least about 30 μg/ml, or more.

The level of secretion of the Wnt3A polypeptide into the serum-free culture medium may be at least about 10 ng/ml. The level of secretion of the Wnt3A polypeptide into the serum-free culture medium may be at least about 25 ng/ml. The level of secretion of the Wnt3A polypeptide into the serum-free culture medium may be at least about 50 ng/ml. The level of secretion of the Wnt3A polypeptide into the serum-free culture medium may be at least about 75 ng/ml. The level of secretion of the Wnt3A polypeptide into the serum-free culture medium may be at least about 100 ng/ml. The level of secretion of the Wnt3A polypeptide into the serum-free culture medium may be at least about 250 ng/ml. The level of secretion of the Wnt3A polypeptide into the serum-free culture medium may be at least about 500 ng/ml. The level of secretion of the Wnt3A polypeptide into the serum-free culture medium may be at least about 750 ng/ml. The level of secretion of the Wnt3A polypeptide into the serum-free culture medium may be at least about 1 μg/ml. The level of secretion of the Wnt3A polypeptide into the serum-free culture medium may be at least about 1.1 μg/ml. The level of secretion of the Wnt3A polypeptide into the serum-free culture medium may be at least about 1.25 μg/ml. The level of secretion of the Wnt3A polypeptide into the serum-free culture medium may be at least about 1.5 μg/ml. The level of secretion of the Wnt3A polypeptide into the serum-free culture medium may be at least about 1.75 μg/ml. The level of secretion of the Wnt3A polypeptide into the serum-free culture medium may be at least about 2.5 μg/ml. The level of secretion of the Wnt3A polypeptide into the serum-free culture medium may be at least about 5 μg/ml. The level of secretion of the Wnt3A polypeptide into the serum-free culture medium may be at least about 7.5 μg/ml. The level of secretion of the Wnt3A polypeptide into the serum-free culture medium may be at least about 10 μg/ml. The level of secretion of the Wnt3A polypeptide into the serum-free culture medium may be at least about 15 μg/ml. The level of secretion of the Wnt3A polypeptide into the serum-free culture medium may be at least about 20 μg/ml. The level of secretion of the Wnt3A polypeptide into the serum-free culture medium may be at least about 25 μg/ml. The level of secretion of the Wnt3A polypeptide into the serum-free culture medium may be at least about 30 μg/ml.

In some embodiments, the C-terminus of the expressed and secreted Wnt polypeptide is truncated by between 5 to 40 amino acids. In some instances, the C-terminus of the expressed and secreted Wnt polypeptide is truncated by between 5 to 35 amino acids, between 10 to 35 amino acids, between 10 to 33 amino acids, between 10 to 30 amino acids, between 15 to 33 amino acids, between 15 to 30 amino acids, between 20 to 35 amino acids, between 20 to 33 amino acids, between 20 to 30 amino acids, between 25 to 33 amino acids or between 25 to 30 amino acids.

In some embodiments, the C-terminus of the expressed and secreted Wnt polypeptide is truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or more amino acids, and may be additionally truncated at the N or C terminus, provided that the protein maintains biological activity. In some embodiments the Wnt polypeptide is truncated by 5 amino acids. In some embodiments the Wnt polypeptide is truncated by 10 amino acids. In some embodiments the Wnt polypeptide is truncated by 15 amino acids. In some embodiments the Wnt polypeptide is truncated by 20 amino acids. In some embodiments the Wnt polypeptide is truncated by 25 amino acids. In some embodiments the Wnt polypeptide is truncated by 30 amino acids. In some embodiments the Wnt polypeptide is truncated by 33 amino acids.

In some instances, the Wnt polypeptide is Wnt3A polypeptide. In some embodiments, the C-terminus of the expressed and secreted Wnt3A polypeptide is truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or more amino acids, and may be additionally truncated at the N or C terminus, provided that the protein maintains biological activity. In some embodiments the Wnt3A polypeptide is truncated by 5 amino acids. In some embodiments the Wnt3A polypeptide is truncated by 10 amino acids. In some embodiments the Wnt3A polypeptide is truncated by 15 amino acids. In some embodiments the Wnt3A polypeptide is truncated by 20 amino acids. In some embodiments the Wnt3A polypeptide is truncated by 25 amino acids. In some embodiments the Wnt3A polypeptide is truncated by 30 amino acids. In some embodiments the Wnt3A polypeptide is truncated by 33 amino acids.

In some embodiments, the Wnt3A polypeptide has a sequence of at least 70%, 80%, 85%, 90%, 95%, 96%, 97%, 98%, or 99% sequence identity to SEQ ID NO:1. In some embodiments, the Wnt3A polypeptide has a sequence of at least 70% sequence identity to SEQ ID NO:1. In some embodiments, the Wnt3A polypeptide has a sequence of at least 80% sequence identity to SEQ ID NO:1. In some embodiments, the Wnt3A polypeptide has a sequence of at least 85% sequence identity to SEQ ID NO:1. In some embodiments, the Wnt3A polypeptide has a sequence of at least 90% sequence identity to SEQ ID NO:1. In some embodiments, the Wnt3A polypeptide has a sequence of at least 95% sequence identity to SEQ ID NO:1. In some embodiments, the Wnt3A polypeptide has a sequence of at least 96% sequence identity to SEQ ID NO:1. In some embodiments, the Wnt3A polypeptide has a sequence of at least 97% sequence identity to SEQ ID NO:1. In some embodiments, the Wnt3A polypeptide has a sequence of at least 98% sequence identity to SEQ ID NO:1. In some embodiments, the Wnt3A polypeptide has a sequence of at least 99% sequence identity to SEQ ID NO:1.

In some embodiments the Wnt3A polypeptide has a sequence of at least 70%, 80%, 85%, 90%, 95%, 96%, 97%, 98%, or 99% sequence identity to SEQ ID NO: 2. In some embodiments, the Wnt3A polypeptide has a sequence of at least 70% sequence identity to SEQ ID NO: 2. In some embodiments, the Wnt3A polypeptide has a sequence of at least 80% sequence identity to SEQ ID NO: 2. In some embodiments, the Wnt3A polypeptide has a sequence of at least 85% sequence identity to SEQ ID NO: 2. In some embodiments, the Wnt3A polypeptide has a sequence of at least 90% sequence identity to SEQ ID NO: 2. In some embodiments, the Wnt3A polypeptide has a sequence of at least 95% sequence identity to SEQ ID NO: 2. In some embodiments, the Wnt3A polypeptide has a sequence of at least 96% sequence identity to SEQ ID NO: 2. In some embodiments, the Wnt3A polypeptide has a sequence of at least 97% sequence identity to SEQ ID NO: 2. In some embodiments, the Wnt3A polypeptide has a sequence of at least 98% sequence identity to SEQ ID NO: 2. In some embodiments, the Wnt3A polypeptide has a sequence of at least 99% sequence identity to SEQ ID NO: 2.

### Wnt Polypeptide Composition and Formulation

Compositions are provided herein wherein the biologically active Wnt polypeptide secreted into minimal serum media (e.g., a serum-free media such as a serum-free, chemically defined media) or in a pharmaceutically acceptable excipient is at a concentration of at least about 0.1 μg/ml; at least about 0.25 μg/ml; at least about 0.5 μg/ml; at least about 0.75 μg/ml; at least about 1 μg/ml; at least about 2.5 μg/ml; at least about 5 μg/ml; at least about 7.5 μg/ml; at least about 10 μg/ml; at least about 25 μg/ml; at least about 30 μg/ml; at least about 50 μg/ml; at least about 75 μg/ml; at least about 100 μg/ml; at least about 250 μg/ml; at least about 500 μg/ml; at least about 750 μg/ml; at least about 1 mg/ml; at least about 2.5 mg/ml; at least about 5 mg/ml; at least about 7.5 mg/ml; at least about 10 mg/ml; at least about 25 mg/ml; at least about 50 mg/ml; at least about 75 mg/ml; at least about 100 mg/ml; or more.

In some embodiments, the protein produced by the methods and culture systems of the invention is incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, or liquid forms, such as tablets, capsules, powders, granules, ointments, solutions, suppositories, injections, inhalants, gels, microspheres, etc. As such, administration of the protein and/or other compounds can be achieved in various ways. The protein and/or other compounds may be systemic after administration or may be localized by virtue of the formulation, or by the use of an implant that acts to retain the active dose at the site of implantation.

In pharmaceutical dosage forms, the protein and/or other compounds may be administered in the form of their pharmaceutically acceptable salts, or they may also be used alone or in appropriate association, as well as in combination with other pharmaceutically active compounds. The agents may be combined to provide a cocktail of activities. The following methods and excipients are exemplary and are not to be construed as limiting the invention.

Pharmaceutical formulations may be provided in a unit dosage form, where the term “unit dosage form,” refers to physically discrete units suitable as unitary dosages for human subjects, each unit containing a predetermined quantity of protein in an amount calculated sufficient to produce the desired effect in association with a pharmaceutically acceptable diluent, carrier or vehicle. The specifications for the unit dosage forms of the present invention depend on the particular composition employed and the effect to be achieved, and the pharmacodynamics associated with the composition in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Depending on the patient or patient sample and condition being treated and on the administration route, the protein may be administered to a patient sample in a dosage of about 0.001 μg to about 10 μg or in a patient in a dosage of about 0.001 μg/kg to about 10 μg/kg body weight (per day).

Those of skill will readily appreciate that dose levels can vary as a function of the specific enzyme, the severity of the symptoms and the susceptibility of the subject to side effects. Some of the proteins are more potent than others. Preferred dosages for a given enzyme are readily determinable by those of skill in the art by a variety of means. A preferred means is to measure the physiological potency of a given compound.

The compositions of the invention can be used for prophylactic as well as therapeutic purposes. As used herein, the term “treating” refers both to the prevention of disease and the treatment of a disease or a pre-existing condition and more generally refers to the enhancement of Wnt3A activity at a desired tissue, site, timing, etc. The invention provides a significant advance in the treatment of ongoing disease, and helps to stabilize and/or improve the clinical symptoms of the patient. Such treatment is desirably performed prior to loss of function in the affected tissues but can also help to restore lost function or prevent further loss of function. Evidence of therapeutic effect may be any diminution in the severity of disease or improvement in a condition, e g enhanced bone healing, etc. The therapeutic effect can be measured in terms of clinical outcome or can be determined by biochemical tests. Alternatively, one can look for a reduction in symptoms of a disease.

In other embodiments of the invention, cell compositions are provided, where the cells comprise an expression vector comprising a C-terminal truncated Wnt3A protein comprising a signal sequence for secretion, which can be the native Wnt3A signal sequence or a heterologous signal sequence, operably linked to a promoter. In some embodiments the cells are CHO cells (e.g., CHO-S cells or CHO-K1 cells). In some embodiments the cells are provided as a composition comprising serum-free culture medium. In other embodiments the cells are frozen and viable, and are optionally provided in aliquots suitable for seeding cultures.

Cells may be provided in a container, e.g. frozen aliquots, at concentrations of from about 10³ cells/ml, 10⁴ cells/ml, 10⁵ cells/ml, 10⁶ cells/ml, 10⁷ cells/ml, up to about 10⁸ cells/ml or more. Cells can be frozen in any suitable medium to maintains the viability of the cells, and may include DMSO. Cell compositions can be provided in a GMP format for example compositions useful in a master cell bank or working cell bank, which are derived from a single host cell under defined conditions and cloning history, then dispensed into multiple containers.

In some embodiments, the specific activity of a Wnt polypeptide in a composition is measured by determining the level of activity in a functional assay, e.g. stabilization of β-catenin, promoting growth of stem cells, etc., quantitating the amount of Wnt polypeptide present in a non-functional assay, e.g. immunostaining, ELISA, western blot, quantitation on coomasie or silver stained gel, etc., and determining the ratio of biologically active Wnt to total Wnt. Generally, the specific activity as thus defined in a substantially homogeneous composition will be at least about 5% that of the starting material, usually at least about 10% that of the starting material, and may be about 25%, about 50%, about 90% or greater.

Assays for biological activity of Wnt include activation of β-catenin, which can be measured, for example, by serial dilutions of the Wnt composition. An exemplary assay for Wnt biological activity contacts a Wnt composition with cells, e.g. mouse L cells, which is stably transfected with a Wnt-responsive luciferase reporter plasmid and a constitutive LacZ expression construct. The luciferase/beta galactosidase (luc/lac) ratio permits normalization of activity per cell number. The cells are cultured for a period of time sufficient to activate β-catenin, usually at least about 1 hour, and lysed. The cell lysate is analyzed for luc/lac expression level by comparing to the standard curve generated with commercially available Wnt proteins. Other assays include C57MG transformation and induction of target genes in Xenopus animal cap assays.

In some embodiments, the Wnt composition comprises a dose-to-dose uniformity. In some embodiments, the Wnt composition has a dose-to-dose Wnt concentration variation of less than 20%, 15%, 10%, 5%, 4%, 3%, 2%, 1%, 0.5%, 0.1%, or less. In some instances, the Wnt composition has a dose-to-dose Wnt concentration variation of less than 20%. In some instances, the Wnt composition has a dose-to-dose Wnt concentration variation of less than 15%. In some instances, the Wnt composition has a dose-to-dose Wnt concentration variation of less than 10%. In some instances, the Wnt composition has a dose-to-dose Wnt concentration variation of less than 5%. In some instances, the Wnt composition has a dose-to-dose Wnt concentration variation of less than 4%. In some instances, the Wnt composition has a dose-to-dose Wnt concentration variation of less than 3%. In some instances, the Wnt composition has a dose-to-dose Wnt concentration variation of less than 2%. In some instances, the Wnt composition has a dose-to-dose Wnt concentration variation of less than 1%. In some instances, the Wnt composition has a dose-to-dose Wnt concentration variation of less than 0.5%. In some instances, the Wnt composition has a dose-to-dose Wnt concentration variation of less than 0.1%.

In some embodiments, the Wnt compositions are substantially free of a biological contaminants (e.g., microorganisms such as bacteria, viruses, or mycobacteria; or host cells or cell debris). In some instances, the Wnt composition comprises at most 5%, 4%, 3%, 2%, 1%, 0.5%, 0.1%, 0.05%, 0.01%, or less of a biological contaminant.

In some embodiments, the Wnt compositions are substantially free of a chemical contaminant (e.g., one or more buffer components utilized during the purification step and/or during the liposomal reconstitution step). In some instances, the Wnt composition comprises at most 5%, 4%, 3%, 2%, 1%, 0.5%, 0.1%, 0.05%, 0.01%, 0.005%, 0.001%, or less of a chemical contaminant. In some instances, the chemical contaminant comprises ethanol. In some instances, the chemical contaminant comprises a detergent. In some instances, the chemical contaminant comprises a sugar detergent (e.g., n-hexyl-β-D-glucopyranoside, n-heptyl-β-D-glucopyranoside, n-octyl-β-D-glucopyranoside, n-octyl-α-D-glucopyranoside, octyl β-D-1-thioglucopyranoside, n-octyl-β-D-galactopyranoside, n-nonyl-β-D-glucopyranoside, n-decyl-β-D-glucopyranoside, n-dodecyl-β-D-glucopyranoside, or methyl-6-O—(N-heptylcarbamoyl)-α-D-glucopyranoside)

### Methods of Use

In certain embodiments, described herein is a method of enhancing cell survival in a bone graft with a liposomal Wnt polypeptide prepared by a method described above. In some embodiments, the method of enhancing cell survival in a bone graft in a subject in need thereof comprises incubating a sample comprising isolated mammalian bone graft material comprising cells ex-vivo with a composition comprising a liposomal Wnt polypeptide generated by a method described above; and transplanting the enhanced cells into a target site.

In some cases, the cells are incubated for at least 5 minutes, 10 minutes, 15 minutes, 20 minutes, 30 minutes, 1 hour, 2 hours, 3 hours, 6 hours, or more. In some cases, the cells are incubated for at least 5 minutes. In some cases, the cells are incubated for at least 10 minutes. In some cases, the cells are incubated for at least 15 minutes. In some cases, the cells are incubated for at least 20 minutes. In some cases, the cells are incubated for at least 30 minutes. In some cases, the cells are incubated for at least 60 minutes. In some cases, the cells are incubated for at least 2 hours. In some cases, the cells are incubated for at least 6 hours or more.

In some cases, the cells are incubated for no more than 30 minutes, 1 hour, 1.5 hour, 2 hours, 3 hours, 4 hours, 5 hours, 6 hours, or less. In some cases, the cells are incubated for no more than 30 minutes. In some cases, the cells are incubated for no more than 1 hour. In some cases, the cells are incubated for no more than 1.5 hours. In some cases, the cells are incubated for no more than 2 hours. In some cases, the cells are incubated for no more than 3 hours. In some cases, the cells are incubated for no more than 6 hours.

In some cases, the cells are incubated from about 5 minutes to about 6 hours, from about 10 minutes to about 6 hours, from about 30 minutes to about 6 hours, from about 5 minutes to about 3 hours, from about 10 minutes to about 3 hours, from about 15 minutes to about 3 hours, from about 30 minutes to about 3 hours, from about 5 minutes to about 2 hours, from about 10 minutes to about 2 hours, from about 15 minutes to about 2 hours, from about 20 minutes to about 2 hours, from about 30 minutes to about 2 hours, from about 5 minutes to about 1 hour, from about 10 minutes to about 1 hour, from about 15 minutes to about 1 hour, or from about 30 minutes to about 1 hour. In some cases, the cells are incubated from about 5 minutes to about 6 hours. In some cases, the cells are incubated from about 10 minutes to about 6 hours. In some cases, the cells are incubated from about 30 minutes to about 6 hours. In some cases, the cells are incubated from about 5 minutes to about 3 hours. In some cases, the cells are incubated from about 10 minutes to about 3 hours. In some cases, the cells are incubated from about 15 minutes to about 3 hours. In some cases, the cells are incubated from about 20 minutes to about 3 hours. In some cases, the cells are incubated from about 30 minutes to about 3 hours. In some cases, the cells are incubated from about 5 minutes to about 2 hours. In some cases, the cells are incubated from about 10 minutes to about 2 hours. In some cases, the cells are incubated from about 15 minutes to about 2 hours. In some cases, the cells are incubated from about 20 minutes to about 2 hours. In some cases, the cells are incubated from about 30 minutes to about 2 hours. In some cases, the cells are incubated from about 5 minutes to about 1 hours. In some cases, the cells are incubated from about 10 minutes to about 1 hour. In some cases, the cells are incubated from about 15 minutes to about 1 hour. In some cases, the cells are incubated from about 20 minutes to about 1 hour. In some cases, the cells are incubated from about 30 minutes to about 1 hour.

In some cases, the cells are incubated at about room temperature or at about 37° C. In some instances, room temperature comprises a temperature less than 30° C., less than 29° C., less than 28° C., less than 27° C., less than 26° C., less than 25° C., less than 24° C., less than 23° C., or less than 22° C. In some instances, room temperature comprises a temperature from about 20° C. to about 30° C., from about 22° C. to about 28° C., or from about 24° C. to about 26° C. In some instances, room temperature comprises about 22° C., about 23° C., about 24° C., about 25° C., about 26° C., about 27° C., or about 28° C.

In some cases, the cells are incubated at a temperature from about 34° C. to about 39° C. In some cases, the cells are incubated at a temperature from about 35° C. to about 38° C., from about 35° C. to about 37° C., from about 36° C. to about 39° C., from about 36° C. to about 38° C., or from about 36° C. to about 37° C. In some cases, the cells are incubated at a temperature from about 35° C. to about 38° C. In some cases, the cells are incubated at a temperature from about 35° C. to about 37° C. In some cases, the cells are incubated at a temperature from about 36° C. to about 39° C. In some cases, the cells are incubated at a temperature from about 36° C. to about 38° C. In some cases, the cells are incubated at a temperature from about 36° C. to about 37° C. In some cases, the cells are incubated at about 37° C.

In some cases, the cells are incubated at a temperature from about 2° C. to about 8° C., from about 2° C. to about 6° C., from about 4° C. to about 8° C., or from about 2° C. to about 4° C.

In some cases, the enhanced cells comprise enhanced osteogenic capacity relative to unexposed mammalian bone graft material.

In some instances, the cells are obtained by a surgical procedure from a subject. In some cases, the cells are not removed from the surgical site. In additional cases, the cells are not modified genetically, are not expanded in culture, or are further processed such as by centrifugation prior to returning the treated cells to the subject.

In some embodiments, also described herein is a method of enhancing cell survival at a bone defect site with a liposomal Wnt polypeptide prepared by a method described above. In some embodiments, the method of enhancing cell survival at a bone defect site in a subject in need thereof comprises administering to the bone defect site a composition comprising a liposomal Wnt polypeptide generated by a method described above, wherein the liposomal Wnt polypeptide enhances cell survival at the bone defect site. In some cases, the method further comprises administering a dental or orthopedic implant at the bone defect site.

In some cases, the bone defect site is an injury site, for example, site of dental or bone injury, e.g., due to a fracture or a surgical procedure.

In some cases, the bone defect site is a dental defect site, e.g., a site for a dental implant. Dental implants comprise endosteal implants, for placement in the jawbone, which comprises screws, cylinders, or plates; and subperiosteal implants, for placement under gum but on or above the jawbone. In some cases, the dental implant comprises a two-stage implant, which involves an initial surgerical procedure to place an implant into, e.g., the jawbone, followed by a subsequent surgical procedure at a later time point to attach an abutment. In other cases, the dental implant comprises a single-stage dental implant in which the attachment of the abutment to the implant may be achieved without the need of a second surgical procedure.

In some instances, the dental or orthopedic implant is administered to the bone defect site prior to administration of the composition comprising a liposomal Wnt polypeptide. For example, the dental or orthopedic implant is administered to the bone defect site about 1 day, 2 days, 3 days, 4 days, 5 days, 6 days, 7 days, 2 weeks, 30 days, 1 month, 2 months, 3 months, 4 months, 5 months, 6 months, 1 year, or more before administration of the composition comprising a liposomal Wnt polypeptide.

In other instances, the dental or orthopedic implant is administered to the bone defect site after administration of the composition comprising a liposomal Wnt polypeptide. For example, the dental or orthopedic implant can be administered to the bone defect site about 1 day, 2 days, 5 days, 7 days, 2 weeks, 30 days, 1 month, 2 months, 3 months, 4 months, 5 months, 6 months, or more after administration of the composition comprising a liposomal Wnt polypeptide.

In additional instances, the dental or orthopedic implant and the composition comprising a liposomal Wnt polypeptide are administered to the bone defect site simultaneously.

In some cases, the liposomal Wnt polypeptide enhances osseointegration of the dental or orthopedic implant.

### Kits/Article of Manufacture

Disclosed herein, in certain embodiments, are kits and articles of manufacture for use with one or more methods, processes, and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the containers are formed from a variety of materials such as glass or plastic. In some instances, the containers are single-use containers.

The articles of manufacture provided herein contain packaging materials. Examples of packaging materials include, but are not limited to, bottles, tubes, bags, containers, bottles, and any packaging material suitable for a selected formulation and intended mode of administration and treatment.

For example, the container(s) include Wnt polypeptides or liposomal Wnt polypeptides. The container(s) optionally includes vials, e.g., glass vials such as single-use glass vials. The kits further optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL” Generally, the term “about” includes an amount that would be expected to be within experimental error, e.g., ±5%, ±10% or ±15%.

The section headings used herein are for organizational purposes only and are not to be construed as limiting the subject matter described.

Unless defined otherwise, technical and scientific terms used herein have the same meaning as commonly understood by one of ordinary skill in the art to which this invention belongs. Singleton et al, Dictionary of Microbiology and Molecular Biology 2nd ed., J.Wiley & Sons (New York, N.Y. 1994), provides one skilled in the art with a general guide to many of the terms used in the present application.

The methods of the disclosure, as well as tests to determine their efficacy in a particular subject or application, can be carried out in accordance with the teachings herein using procedures standard in the art. Thus, the practice of the present disclosure may employ conventional techniques of molecular biology (including recombinant techniques), microbiology, cell biology, biochemistry and immunology within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction” (Mullis et al., eds., 1994); and “Current Protocols in Immunology” (J. E. Coligan et al., eds., 1991); as well as updated or revised editions of all of the foregoing.

As used herein, compounds which are “commercially available” may be obtained from commercial sources including but not limited to Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), R&D systems, Inc. (Minneapolis Minn.), Riedel de Haen AG (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.), Novabiochem and Argonaut Technology.

Compounds can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

As used herein, minimal serum condition includes serum conditions with reduced serum presence, for example, about 9%, 8%, 7%, 6%, 5%, 4%, 3%, 2%, 1.5%, 1%, 0.5%, 0.25%, 0.2%, 0.1%, 0.05% serum, or less. In some instances, the minimal serum condition comprises from 9% to 0%, from 5% to 0.05%, from 5% to 0.1%, from 5% to 0.25%, from 4% to 0.05%, from 4% to 0.1%, from 4% to 0.2%, from 3% to 0.05%, from 3% to 0.1%, from 3% to 0.2%, from 3% to 0.25%, from 2% to 0.05%, from 2% to 0.01%, from 2% to 0.25%, or from 2% to 0.5% serum. In some instances, the minimal serum condition comprises reduced-serum media, protein-free media, chemically defined media, or serum-free media. In some cases, reduced-serum media comprises about 1% to about 5% serum (e.g., fetal bovine serum). In some cases, protein-free media does not contain any proteins or components of animal origin, but sometimes contain peptides and/or polypeptides obtained from plant hydrolysates. In some cases, chemically defined media comprises recombinant proteins and/or hormones (e.g., recombinant albumin and insulin, and chemically defined lipids) and does not contain fetal bovine serum, bovine serum albumin or human serum albumin. In some cases, a chemically defined media is a protein-free, chemically defined media, which comprises low molecular weight constituents and sometimes also contain synthetic peptides and/or hormones. In some cases, a chemically defined media is a peptide-free, protein-free chemically defined media. In some cases, serum-free media (or defined media) comprises undefined animal-derived products such as serum albumin, hydrolysates, growth factors, hormones, carrier proteins, and attachment factors. In some embodiments, the minimal serum condition used herein refers to a media condition comprising less than 9%, 8%, 7%, 6%, 5%, 4%, 3%, 2%, 1.5%, 1%, 0.5%, 0.25%, 0.2%, 0.1%, or 0.05% serum. In some embodiments, the minimal serum condition used herein refers to a media condition comprising from 9% to 0%, from 5% to 0.05%, from 5% to 0.1%, from 5% to 0.25%, from 4% to 0.05%, from 4% to 0.1%, from 4% to 0.2%, from 3% to 0.05%, from 3% to 0.1%, from 3% to 0.2%, from 3% to 0.25%, from 2% to 0.05%, from 2% to 0.01%, from 2% to 0.25%, or from 2% to 0.5% serum. In some embodiments, the minimal serum condition used herein refers to a reduced-serum media condition. In some embodiments, the minimal serum condition used herein refers to protein-free media condition. In some embodiments, the minimal serum condition used herein refers to a chemically defined media condition. In some embodiments, the minimal serum condition as used herein refers to a serum-free media condition. In some embodiments, the minimal serum condition as used herein refers to a serum-free, chemically defined media condition.

### EXAMPLES

These examples are provided for illustrative purposes only and not to limit the scope of the claims provided herein.

### Example 1—General Methodology

Plasmid DNA Scale-Up

Each DNA expression construct was scaled up to the appropriate amount for transfection. The plasmid DNA was run on agarose gel for quality assessment and sequence confirmed before proceeding to transfection.

CHO Cells Transient Transfection

Suspension CHO cells were seeded in a shake flask and were expanded using CD OptiCHO media supplemented with 4 mM GlutaMAX. On the day of transfection, the expanded cells were seeded into a new flask with fresh medium. Each DNA construct was transiently transfected into the CHO cells using the MaxCyte STX Scalable Transfection System with the OC-400 processing assembly. The cells were temperature shifted from 37° C. to 32° C. one day after the transfection and maintained as a batch-fed culture with 3.4% MaxCyte feed added daily until the end of the production run on day 7. Table 2 illustrates transfection details of an illustrative Wnt3A variant.

TABLE 2

| | Amount of | Cell density at | Cell viability | Cell density | Cell viability |
|---|---|---|---|---|---|
| Construct | DNA used | transfection | at transfection | at harvest | at harvest |
| Wnt3A Variant 4 | 0.2 mg | 4.0 × 10⁶ | 95% | 7.18 × 10⁶ | 68% |
| (His-hWnt3A) | | cells/mL | | cells/mL | |
| (SEQ ID NO: 13) | | | | | |

IMAC Purification of His-Tagged Protein

The conditioned media from the transient production run was harvested and clarified by centrifugation and filtration. The supernatant was loaded over an Immobilized Metal (Nickel) Affinity Chromatography (IMAC) column, pre-equilibrated with binding buffer [e.g., 20 mM Tris-HCl, 500 mM NaCl, 1% CHAPS]. Washing buffer [e.g., 20 mM Tris-HCl, 500 mM NaCl, 1% CHAPS] containing 40 mM imidazole was passed through the column until OD280 value (NanoDrop, Thermo Scientific) was close to zero. The target protein was eluted with a linear gradient of increasing imidazole concentration up to 0.5 M. The eluate was collected in fractions.

CE-SDS Analysis

CE-SDS analysis of each eluted fraction was performed using LabChip GXII (Perkin Elmer) and analyzed.

### Example 2—Co-Expression of a Wnt3A Polypeptide with a Frizzled-8 Fusion Protein

Frizzled-8 fusion protein (SEQ ID NO: 5) is a soluble protein that comprises the first 151 amino acid residues of Frizzled-8 linked to the Fc region of IgG1 through a poly-Gly linker. In some instances, co-expression of the Frizzled-8 fusion protein with Wnt3A increases the expression of Wnt3A and decreases Wnt3A aggregation. In some cases, the Wnt3A-Frizzled-8 complex inactivates Wnt3A and stabilizes Wnt3A. Removal of the Frizzled-8 fusion protein from the complex reactivates Wnt3A.

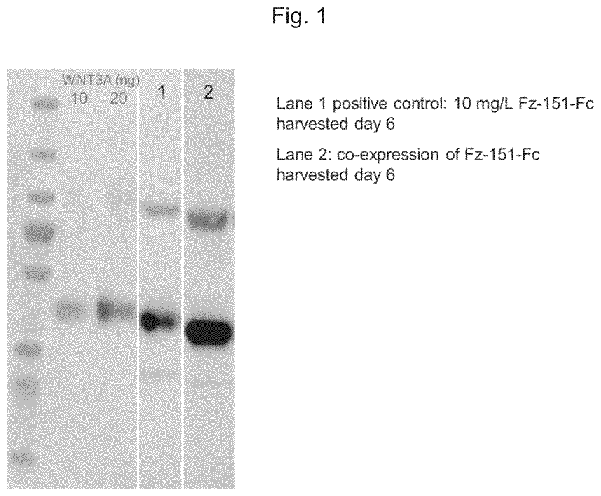

FIG. 1 illustrates a comparison study of Wnt3A expression in the presence of exogenous Frizzled-8 fusion protein (Fz-151-Fc) or in the presence of co-expressed Frizzled-8 fusion protein (Fz-151-Fc). As illustrated in lane 2, the expression of Wnt3A co-expressed with Frizzled-8 fusion protein increased by about 5-fold relative to the Wnt3A expression in the presence of exogenous Frizzled-8 fusion protein. Lane 1 shows the expression of Wnt3A in the presence of exogenous Frizzled-8 fusion protein.

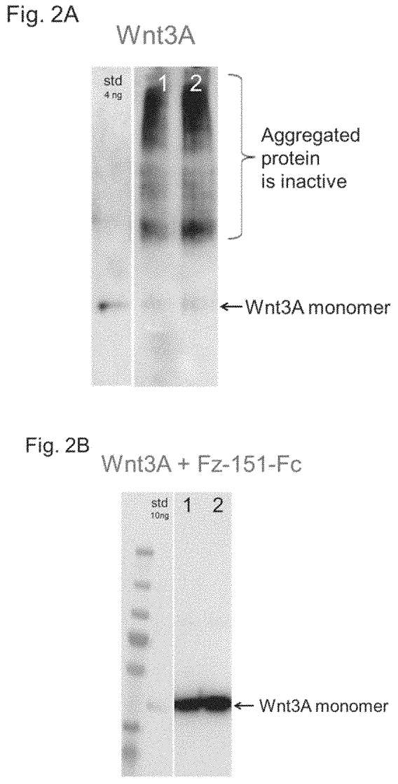

FIG. 2 shows co-expression of Frizzled-8 fusion protein (Fz-151-Fc) reduces Wnt3A aggregation and further increases the amount of Wnt3A monomer. The Wnt3A polypeptide was produced from a stable cell line.

FIG. 3 illustrates four exemplary purification strategies described herein.

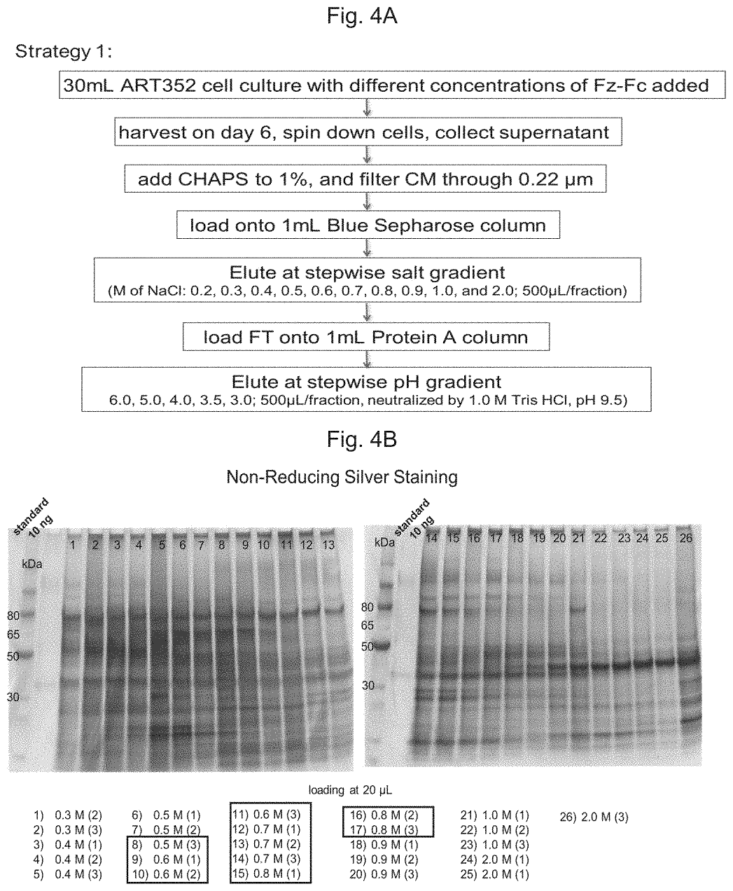

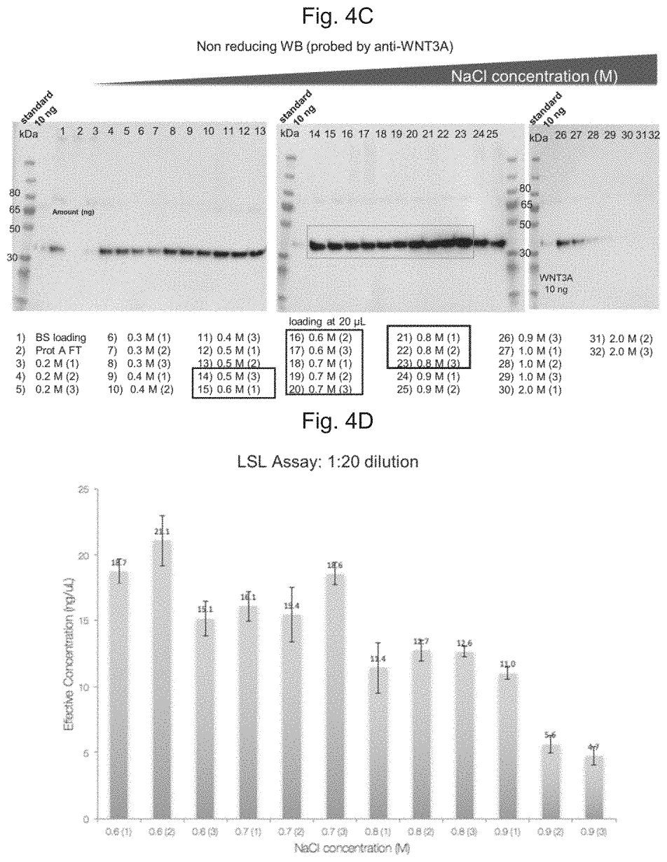

FIG. 4 illustrates purification details of strategy 1. FIG. 4A shows an exemplary purification scheme for Strategy 1. FIG. 4B shows the silver staining of the various fractions. The condition is a non-reducing condition. FIG. 4C shows a western blot analysis of the various fractions to determine the presence and concentration of Wnt3A polypeptide. FIG. 4D illustrates the activity of the Wnt3A polypeptide in a LSL assay.

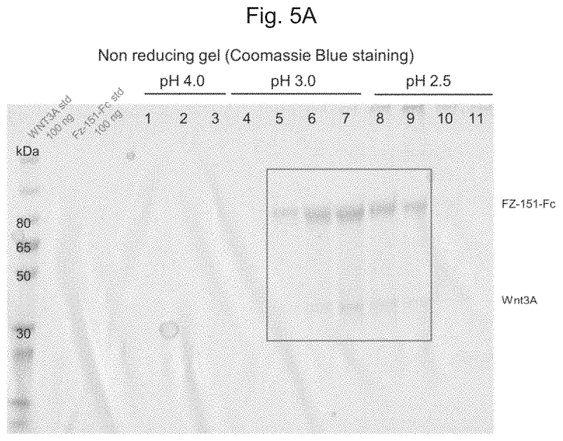

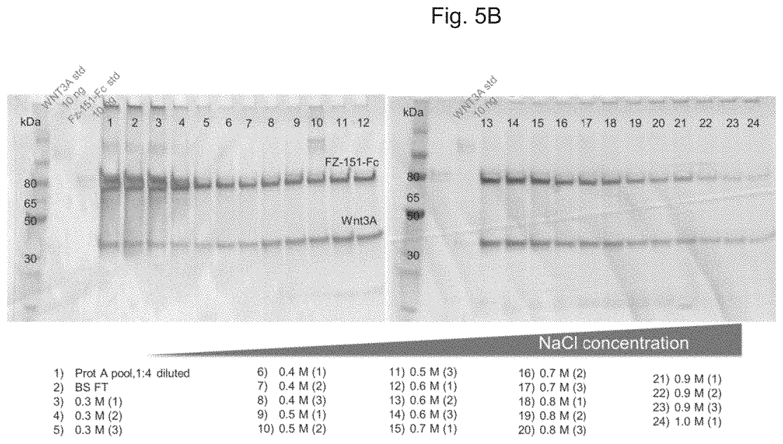

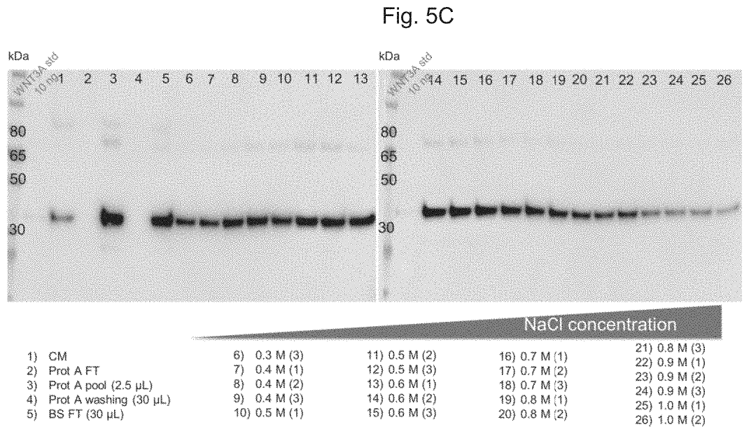

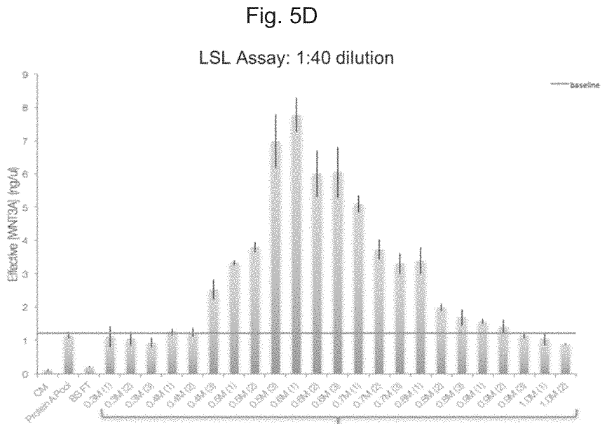

FIG. 5 illustrates purification details for strategy 2. FIG. 5A illustrates a Coomassie staining of Protein A fractions. FIG. 5B shows the silver staining of the various fractions. FIG. 5C shows a western blot analysis of the various fractions to determine the presence and concentration of Wnt3A polypeptide. FIG. 5D illustrates the activity of the Wnt3A polypeptide in a LSL assay.

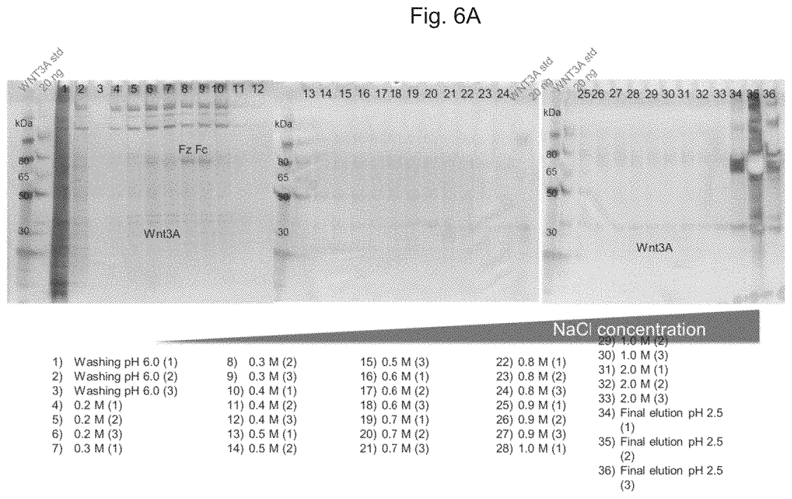

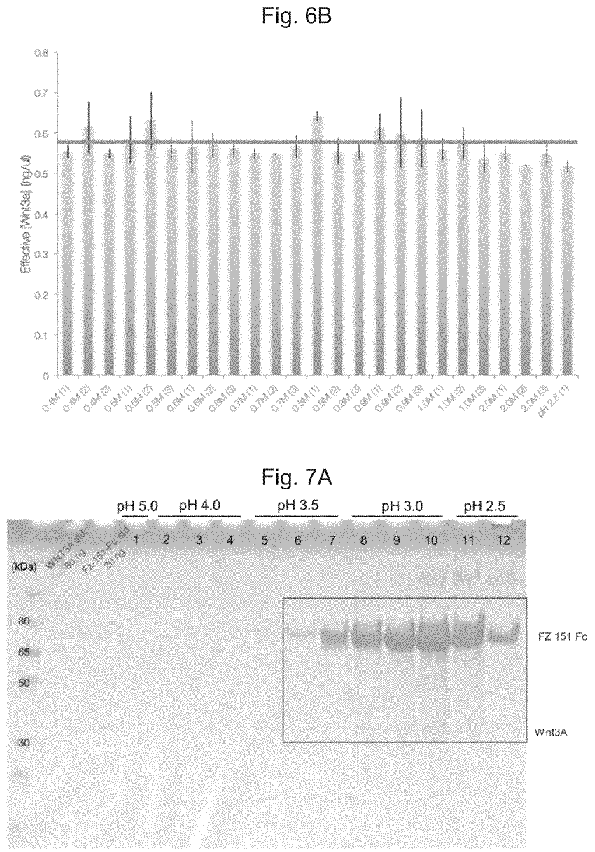

FIG. 6 illustrates purification details for strategy 3. FIG. 6A shows the silver staining of the various fractions. FIG. 6B illustrates the activity of the Wnt3A polypeptide in a LSL assay.

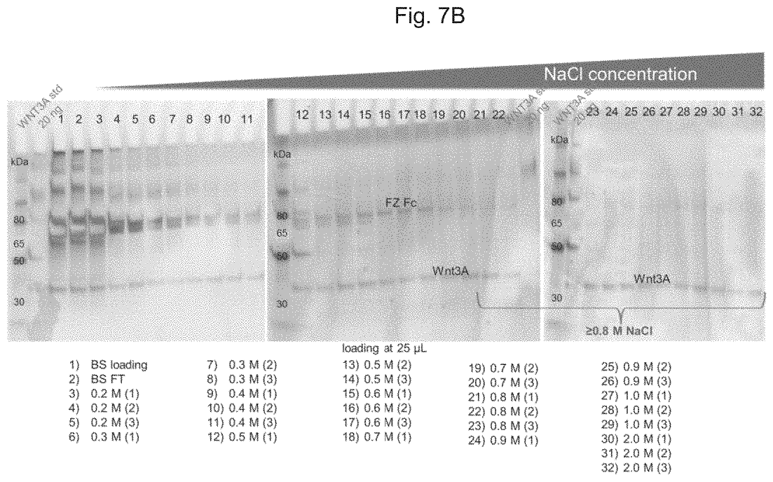

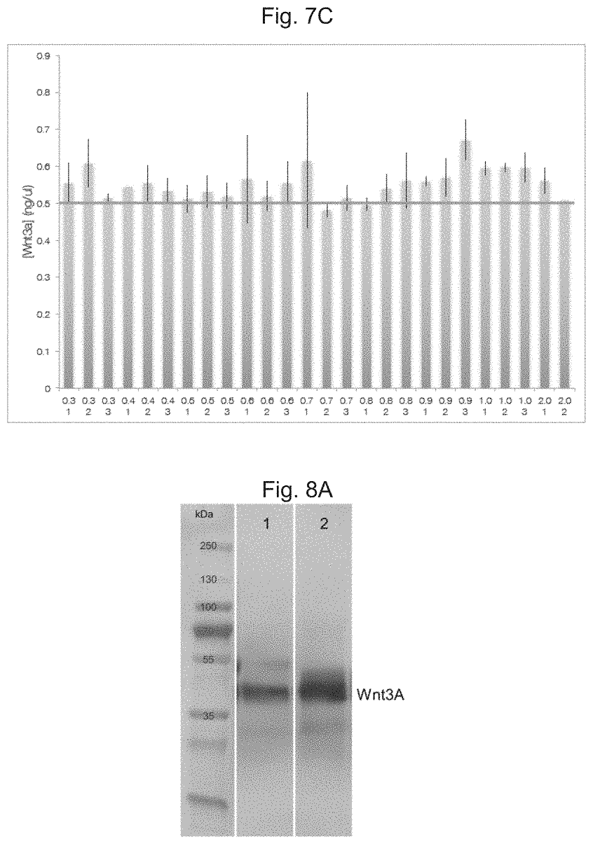

FIG. 7A-FIG. 7C illustrate purification details for strategy 4. FIG. 7A shows a Coomassie staining of Protein A fractions. FIG. 7B shows the silver staining of the various fractions. FIG. 7C illustrates the activity of the Wnt3A polypeptide in a LSL assay.

### Example 3—Co-Expression of a Wnt3A Polypeptide with a Chaperone

Wntless is an intracellular chaperone that binds with functional, lipid-modified Wnt polypeptide and is required for transport of Wnt polypeptide from the golgi apparatus to the cell surface.

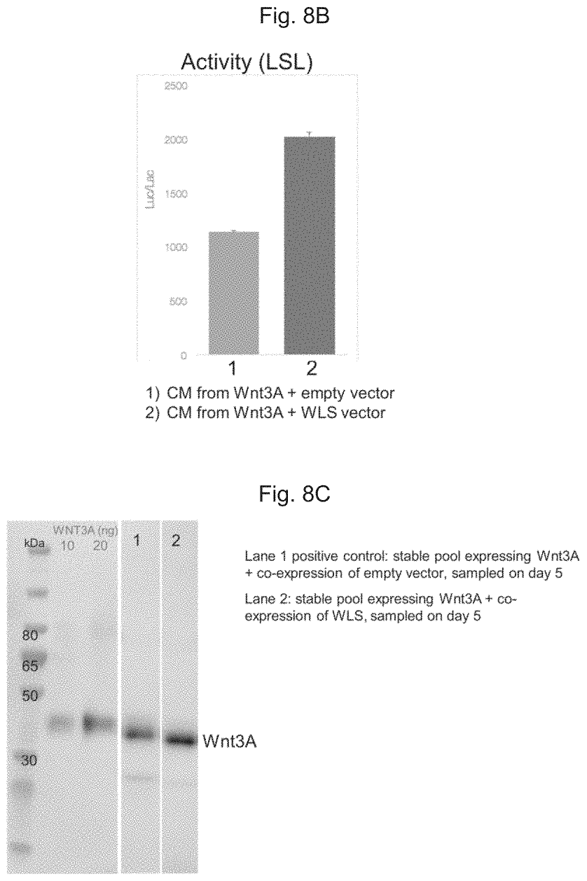

FIG. 8 illustrates co-expression of a Wnt3A polypeptide with Wntless (WLS). FIG. 8A shows an increase in Wnt3A expression in the presence of co-expressed Wntless. FIG. 8B shows the activity of Wnt3A polypeptide in a LSL assay. FIG. 8C shows expression of Wnt3A in a stable cell line.

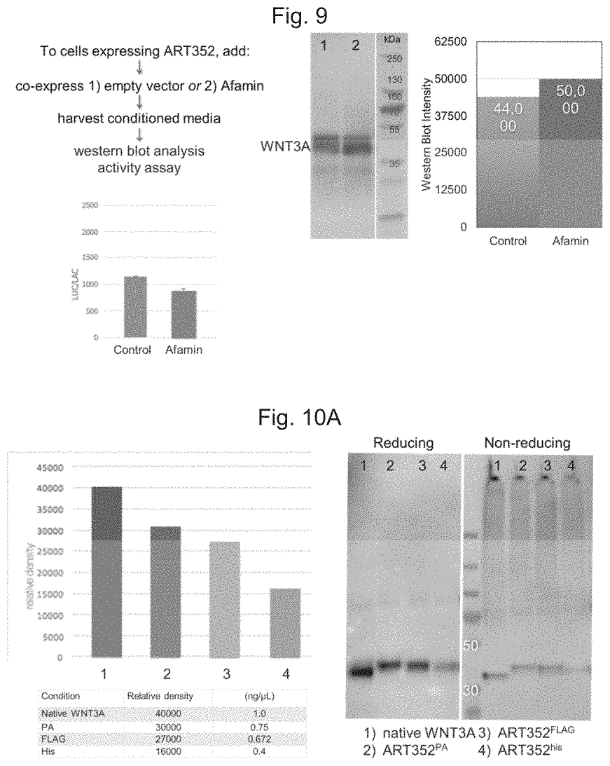

FIG. 9 illustrates co-expression of Wnt3A with Afamin. In some instances, co-expression of Afamin increases Wnt3A concentration by about 10%.

### Example 4—Expression and Production of a Tagged Wnt3A Polypeptide

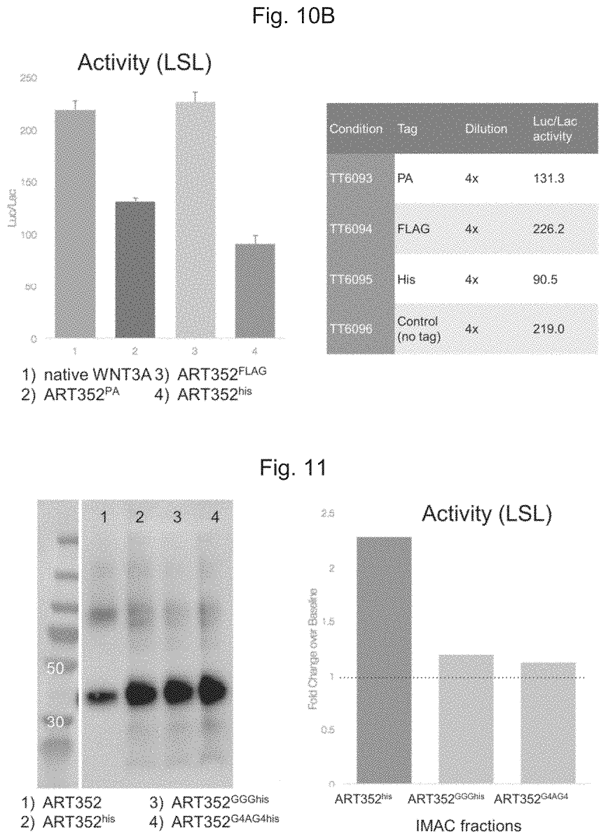

FIG. 10 illustrates the expression and activity of three exemplary Wnt3A polypeptides tagged with: PA, FLAG, and His-tag, respectively. FIG. 10A illustrates the concentration of the secreted tagged Wnt3A polypeptides. FIG. 10B shows the activity of Wnt3A polypeptides in a LSL assay.

FIG. 11 shows the activity of Wnt3A variants (ART352^{his} variants) comprising different His-tag-linker constructs.

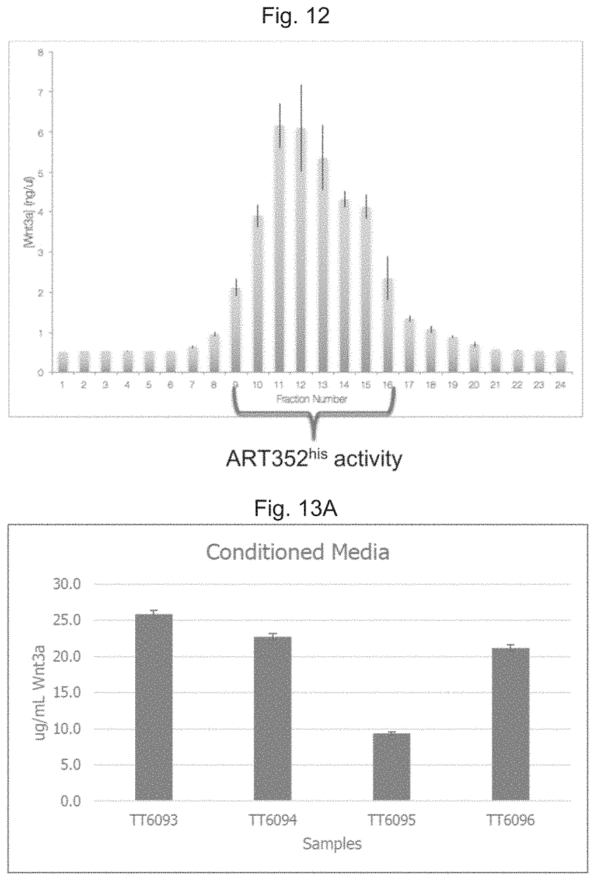

FIG. 12 shows the activity of the various fractions of the Wnt3A variant-ART352^{his} from a Ni-NTA column.

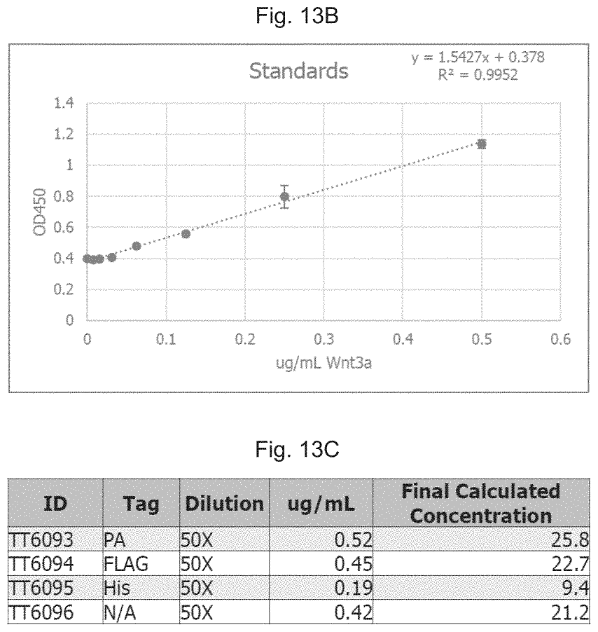

FIGS. 13-15 show the expression and production of N-terminally tagged Wnt3A polypeptides. The Wnt3A polypeptide constructs used herein for FIGS. 13-15 are:

TT6093: PA-TEV-Wnt3A

TT6094: FLAG-TEV-Wnt3A

TT6095: His-TEV-Wnt3A

TT6096: Wnt3A

Conditioned media was harvested on Day 7.

FIG. 13A-FIG. 13C illustrate the concentration of the N-terminally tagged Wnt3A polypeptides in an ELISA assay.

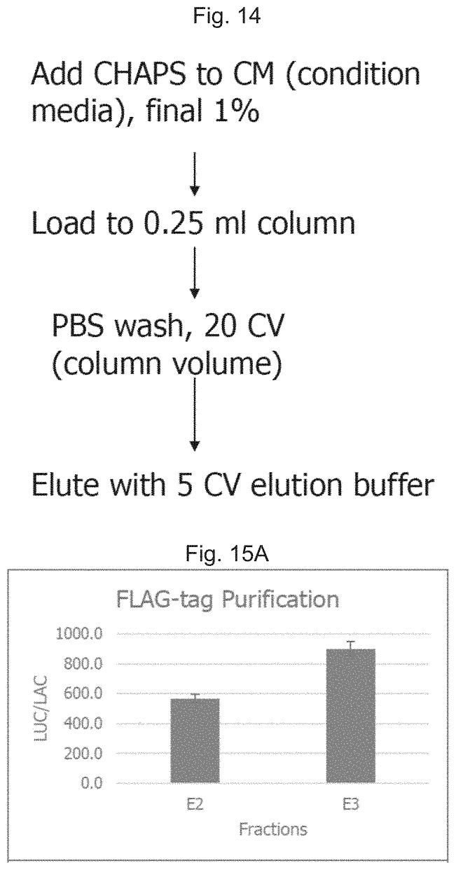

FIG. 14 illustrates a purification scheme for purification of a FLAG-tagged Wnt3A polypeptide: FLAG-TEV-hWnt3A. CHO cells were transiently transfected in 40 mL condition media. CHAPS was added to the condition media at a final concentration of 1%. The solution was then loaded onto a 0.25 mL HM2-agarose column and eluted with 5 column volume of an elution buffer comprising FLAG peptide at 100 μg/mL in 1×PBS buffer and 1% CHAPS.

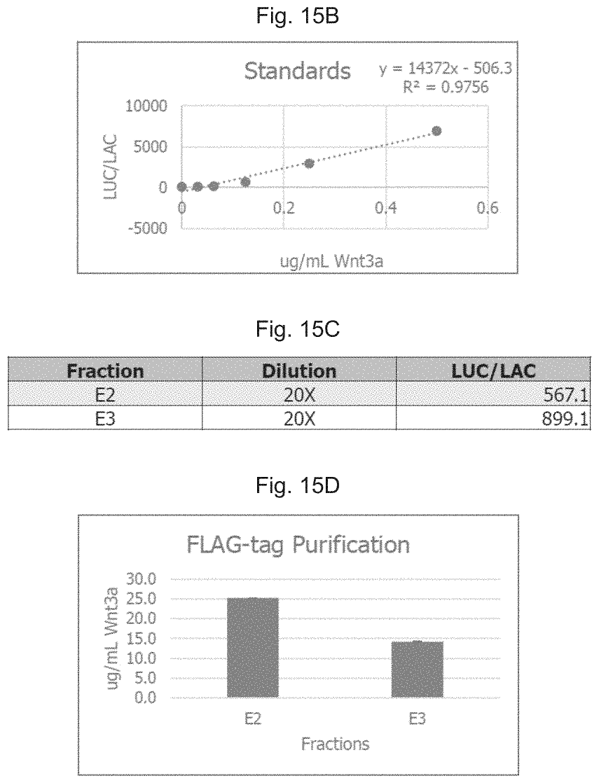

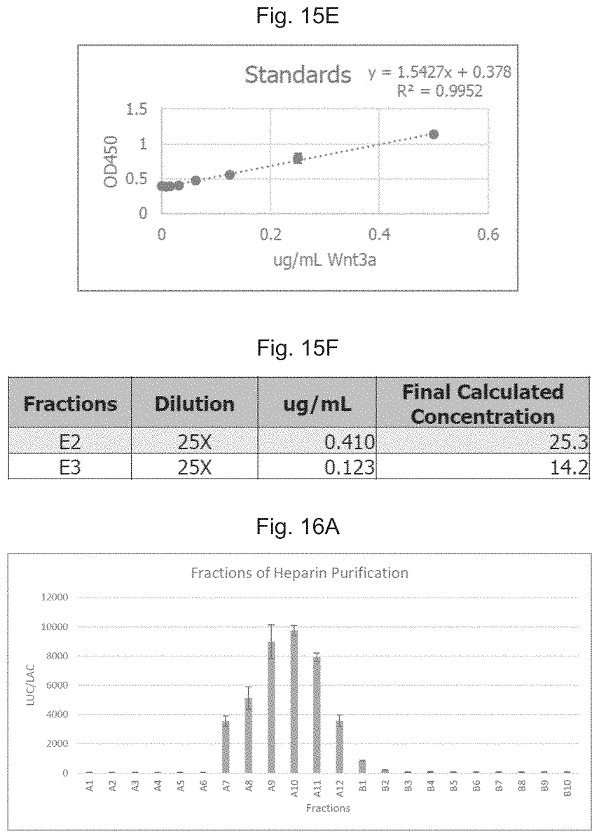

FIG. 15 shows the activity and concentration of the FLAG-tagged Wnt3A polypeptide. FIG. 15A-FIG. 15C show the activity of the Wnt3A polypeptide in a LSL assay. FIG. 15D-FIG. 15F show the concentration of the Wnt3A polypeptide.

### Example 5—Purification of a Wnt3A Polypeptide at Two Different Culture Volumes

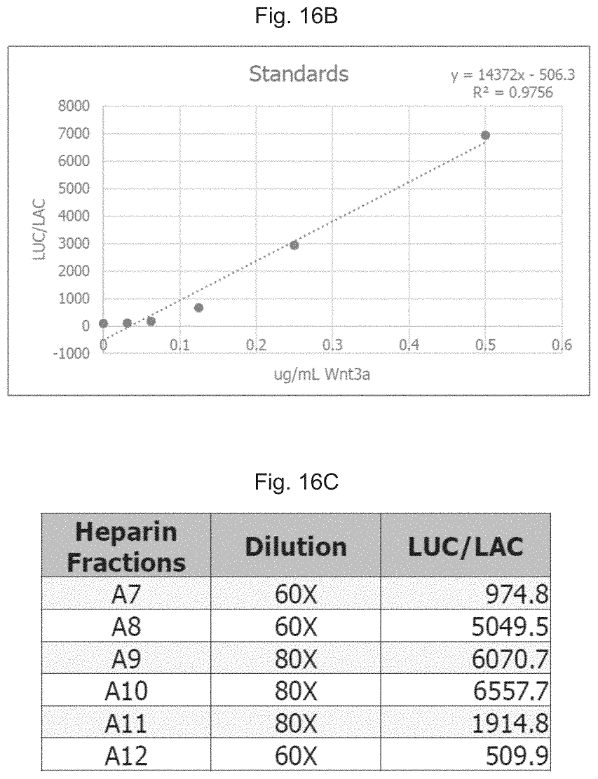

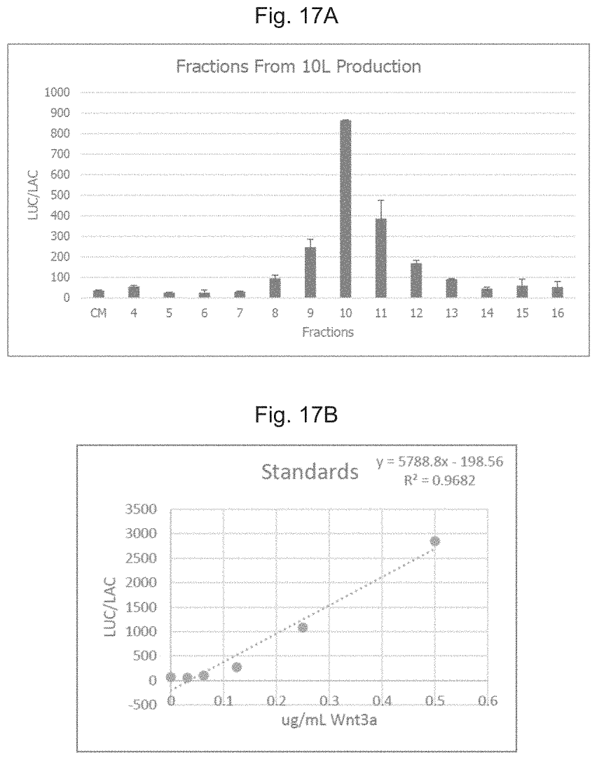

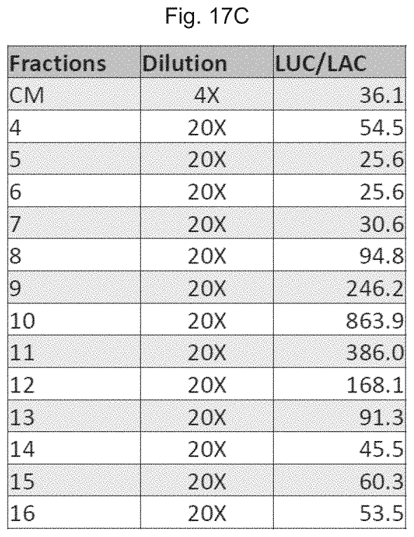

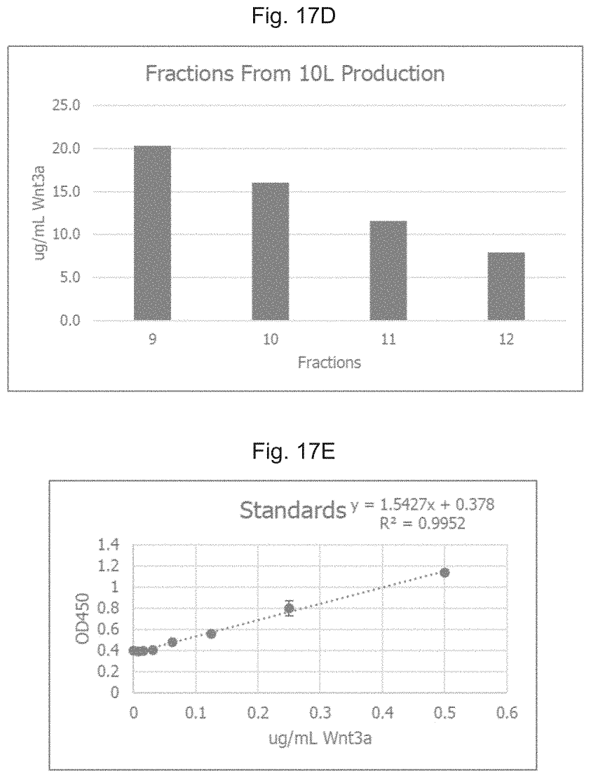

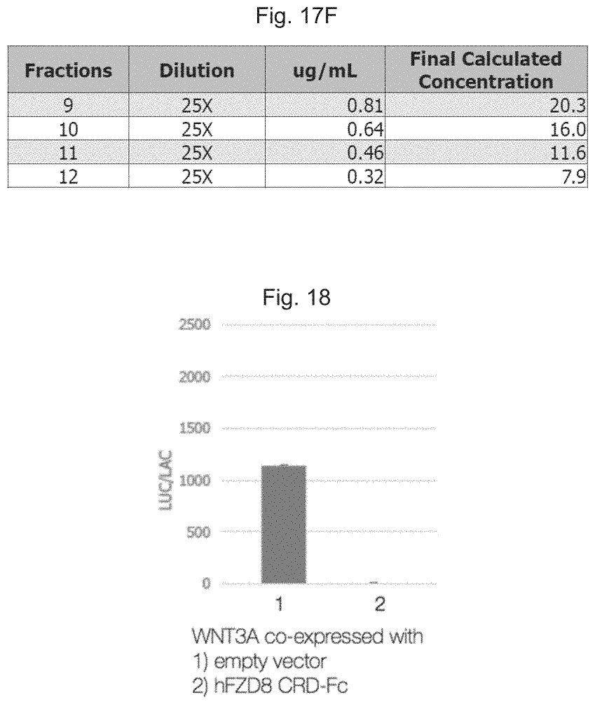

Wnt3A comprising SEQ ID NO: 2 was purified from either a 0.75 L culture or a 10 L culture. The condition media was first loaded onto a 5 mL Blue Sepharose column followed by purification with a Heparin column. FIG. 16 shows the activity of the Wnt3A cultured from the 0.75 L culture. FIG. 17 shows the activity and concentration of Wnt3A cultured from the 10 L culture.

### Example 6—Purification of a Wnt3A Polypeptide with an Exemplary Sugar Detergent OGP

In this experiment, exemplary sugar detergent OGP was utilized both as a competitive antagonist and as a stabilizer to Wnt proteins prior to incubation with a liposome. OGP, also referred to herein as n-octyl-β-D-glucopyranoside, OG, C8Glc, octyl-beta-glucoside, octyl-beta-glucopyranoside, or octyl-beta-D-glucopyranoside, is a non-ionic detergent, which has been shown to interact with the cysteine-rich domain (CRD) of a human Frizzled 5 receptor. In this study, OGP was shown to be able to out-compete binding of Wnt with a fusion Frizzled 8 protein during the purification of the Wnt polypeptide complex, as while as to stabilize the Wnt polypeptide during purification.

CHO cells were engineered to co-express an exemplary truncated Wnt3A polypeptide and a modified human Frizzled 8 protein comprising an Fc-tagged CRD domain (hFZD8 CRD-Fc). Secreted Wnt3A polypeptide forms a soluble complex with hFZD8 CRD-Fc. Activity was not detected for the Wnt3A polypeptide in the complex, based on a LSL cell based assay (FIG. 18).

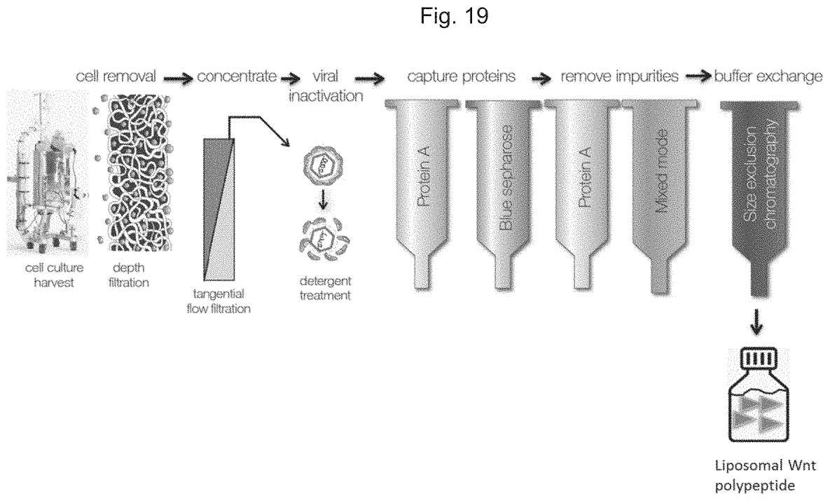

The purification scheme is illustrated in FIG. 19. In brief, Wnt3A polypeptide-hFZD8 CRD-Fc complexes were harvested from condition media and loaded onto a first Protein A column. The pH of the elution buffer is less than about 4.0. The elute from the first Protein A column was incubated with a buffer solution comprising about 1% OGP. The incubated eluate was then loaded onto a Blue Sepharose column to separate the hFZD8 CRD-Fc from the Wnt3A polypeptide. A linear gradient of 0.8-2M NaCl (in the elution buffer which further comprises about 1% OGP) was used to collect the Wnt3A polypeptide. The Wnt3A polypeptide was further subjected to a second Protein A column, followed by a mixed mode column and a size exclusion chromatography column, in tandem, to generate the purified Wnt3A polypeptide.

CHAPS was used as a control.

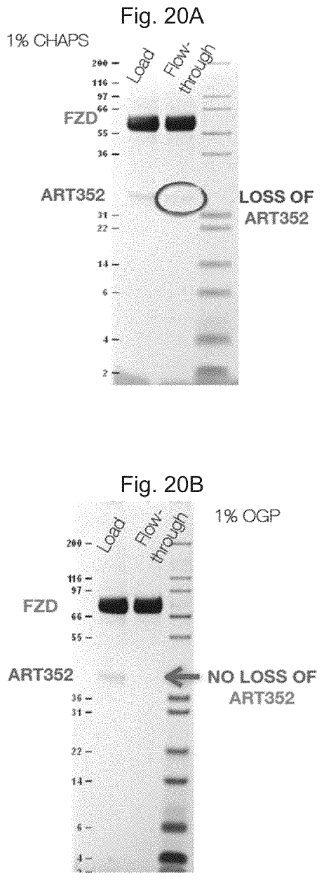

FIG. 20A-FIG. 20B show exemplary gel images of Wnt3A purification with either 1% CHAPS or 1% OGP. As shown in FIG. 20B, replacement of CHAPS with OGP enables more efficient separation of the Wnt3A (ART352)-FZD complex relative to FIG. 20A. Furthermore, the inclusion of OGP stabilizes Wnt3A (ART352) once it was released from interaction with FZD.

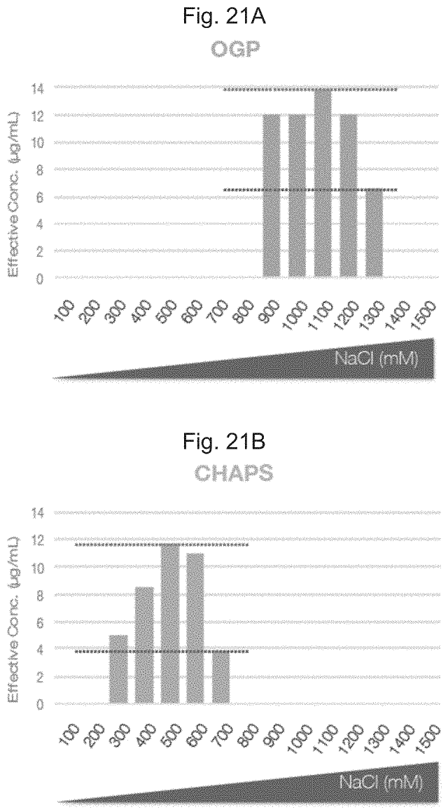

FIG. 21A-FIG. 21B illustrate LSL activity of WNT3A (ART352) eluates in 1% OGP (FIG. 21A) or 1% CHAPS (FIG. 21B).

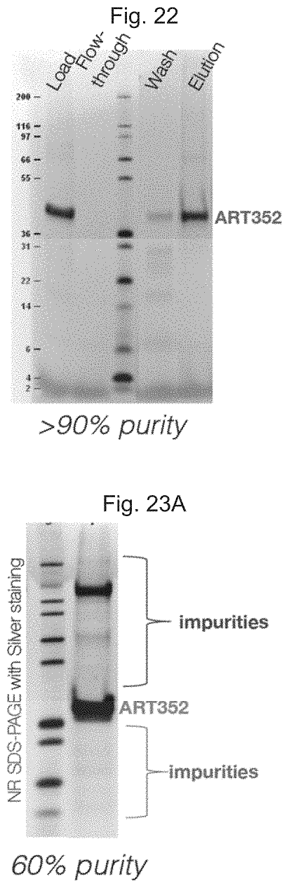

FIG. 22 illustrates an exemplary gel image of purification with a mixed mode column. The purity of the Wnt3A eluate was about greater than 90%.

FIG. 23A-FIG. 23B illustrate Wnt3A polypeptide purified with either buffer comprising 1% CHAPS or 1% OGP. Greater impurities were observed in the solution comprising Wnt3A polypeptide purified with buffer comprising 1% CHAPS (FIG. 23A) than with buffer comprising 1% OGP (FIG. 23B).

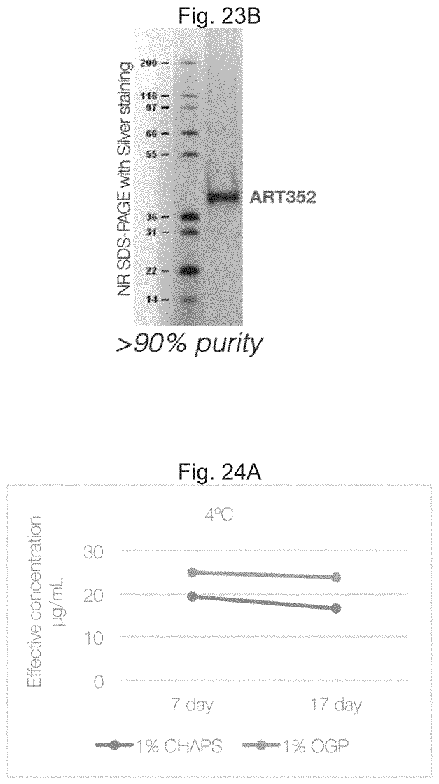

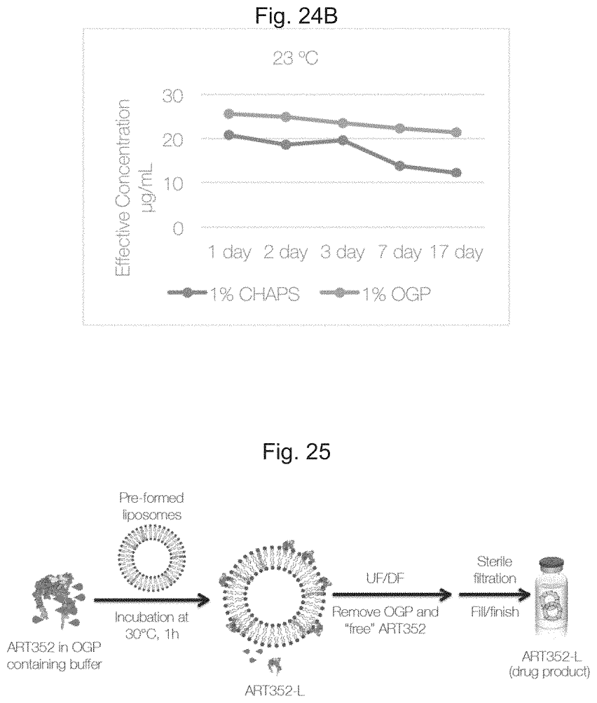

FIG. 24A-FIG. 24B illustrate that OGP stabilizes WNT3A protein at 2 different temperatures, 4° C. (FIG. 24A) and 23° C. (FIG. 24B) in comparison to CHAPS.

FIG. 25 illustrates an exemplary liposomal Wnt3A formulation process.

### Example 7—Comparison of Two Different Wnt3A Manufacturing Processes

The manufacturing process for Wnt3A and an exemplary Wnt3A polypeptide ART352 was compared. Table 3 illustrates the manufacturing details of the respective Wnt3A polypeptides.

| Drug Substance | | |
|---|---|---|
| Property | Wnt3A | ART352 |
| Host Cells | Adherent CHO | CHO adapted to suspension |
| Cell Culture | Contains serum | Chemically-defined, |
| Media | | protein-free |
| Cell Culture | batch | fed-batch |
| Process | | |
| Purification | A single | Multi chromatographic |
| Process | chromatography | steps including a |
| | column | column immobilized with |
| | immobilized | a sulfonated polyaromatic |
| | with a sulfonated | compound, an affinity |
| | polyaromatic | chromatography column, |
| | compound | and a mixed mode column |
| Formulation | A functionally active | A functionally active |
| | Wnt polypeptide in a | Wnt polypeptide in |
| | buffer containing | a buffer containing a |
| | CHAPS | sugar detergent |

Table 4 shows the manufacturing details of L-Wnt3A and an exemplary liposomal Wnt3A polypeptide ART352-L.

| Drug Product Property | L-Wnt3A | ART352-L |
|---|---|---|
| manufacturing process | WNT3A is concentrated with | ART352 is incubated with pre-formed |
| | a centrifugal filter WNT3A is | liposomes at 25-30° C. for 2 hours |
| | incubated with pre-formed | The resulting ART352-L undergoes |
| | liposomes at room | ultrafiltration and diafiltration and is |
| | temperature, overnight | formulated in the final buffer |

Table 5 illustrates the potency and purity differences of the two processes.

| | L-Wnt3A (ng/μL) | ART352-L (ng/μL) |
|---|---|---|
| Potency | 0.68 | 0.82 |
| Purity | ~50% | >90% |

### Example 8—Determination of Potency

Calculation of Potency

Luciferin is converted into oxyluciferin by the luciferase enzyme, and nearly all of the energy released by this reaction is in the form of light that is detected by a plate reader. Because the expression of luciferase is under control of TCF/LEF binding sites, the expression of luciferase is proportional to Wnt activity.

Use a 4-parameter logistic curve-fitting program to generate a standard curve by relative luminescence units (RLUs) against the ART352 (x) concentration expressed μg/mL:

Where: x=the independent variable, i.e. dose

A=Left asymptote

B=curvature hill slope

C=Effective concentration at which a drug gives one-half the maximum response (EC50), μg/mL

D=Right asymptote

The percent specific potency for the control and sample(s) is calculated using the following formula:

$$\frac{EC 50 of STD ( µg \text{/} mL )}{EC 50 of control or sample ( s ) ( µg \text{/} mL )} \times 100 \%$$

Where: EC50=the one-half maximal Effective Concentration

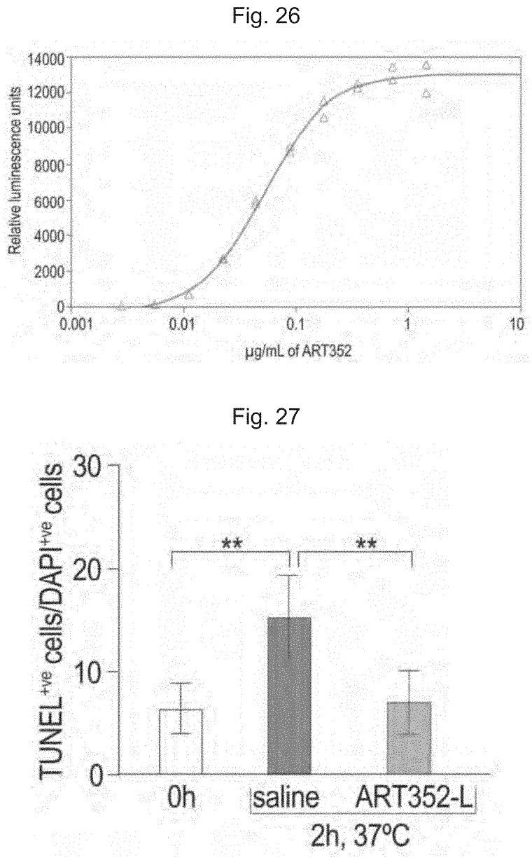

The potency of the exemplary Wnt3A polypeptide ART352 and liposomal Wnt3A polypeptide ART352-L is defined by comparing the readouts of samples to that of a reference standard (see below Reference Standard section), tested at known concentrations. A representative standard curve in the range of 0.003-1.6 μg/mL is shown in FIG. 26.

Potency results for WNT3A/L-WNT3A and for ART352/ART352-L are compared in Table 6.

| Potency | WNT3A | ART352 | ART352 |
|---|---|---|---|
| results | in 40 mL | from first | from second |
| (μg/mL) | batches | 50 L batch | 50 L batch |
| Protein | 0.5 | 5.5 | 17.9 |
| Liposomal | 0.68 | 0.36 | 0.82 |
| formulation | | | |
| of protein | | | |

During manufacturing process development prior to finalization of GMP processes, the first 50 L batch of ART352-L was manufactured using sub-optimal processes compared to the second 50 L batch. Consequently, ART352 and ART352-L from the first batch exhibited lower potency as compared to the corresponding results from ART352 and ART352-L generated from the second batch. These data showed that the LSL assay is suitably sensitive to detect meaningful batch-to-batch differences in potency.

### Example 9—Process Development of Autograft Treatment and Handling

Effect of Solution Condition and Temperature on Cell Viability in an Autograft

Autografts were harvested from the iliac crest. To establish a baseline for apoptosis, a subset of autografts was immediately processed for TUNEL staining (white bar, FIG. 27). This represented a zero ex vivo time point.

The remaining autografts were either placed in saline, or in saline containing ART352-L (effective concentration=0.5 ng/μL). Autografts were incubated for the maximum duration of an ex vivo hold, e.g., 2h, and the maximum temperature, e.g., 37° C. was employed. Cell viability and apoptosis were quantified using TUNEL and DAPI as described in Allen et al., “Morphological and biochemical characterization and analysis of apoptosis,” J Pharmacol Toxicol Methods 37(4):215-228 (1997); and Kapuscinski, J. “Dapi: A DNA-specific fluorescent probe,” Biotechnic & histochemistry: official publication of the Biological Stain Commission. 70(5):220-233 (1995); respectively. These studies show:
Compared to control autografts at zero-time point, autografts held in saline for 2h at 37° C. exhibited significantly more dying cells (FIG. 27).
Compared to the extent of apoptosis in autografts held in saline for 2h at 37° C., ART352-L treated autografts held under the same conditions exhibited significantly fewer dying cells (FIG. 27).

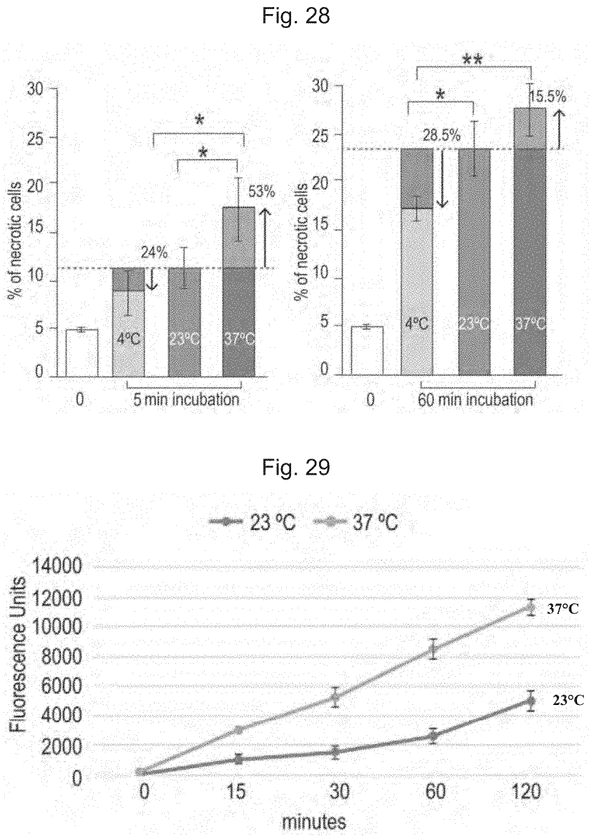

Autografts that were harvested and immediately analyzed (e.g., the zero-time point samples) served as the negative control for TUNEL. The remaining autografts were harvested then placed in saline at 4° C., 23° C., or 37° C. for 5 minutes, or for 60 minutes. Cell viability was quantified using trypan blue exclusion (FIG. 28). Samples held at 23° C. served as the positive control for TUNEL. The autografts were in saline. These data show:
Cell viability at the time of harvest represented the zero-time point, baseline condition (white bars, FIG. 28).
Holding an autograft at 4° C. for 5 minutes increases the amount of necrosis observed at the zero time point by 24% (FIG. 28).
Holding an autograft at 23° C. approximately doubles the number of necrotic cells observed at the zero-time point (FIG. 28). A hold temperature of 23° C. also significantly increases the number of necrotic cells compared to a hold temperature of 4° C. (FIG. 28).
Holding an autograft at 37° C. approximately triples the number of necrotic cells observed at the zero-time point (FIG. 28). A hold temperature of 37° C. also significantly increases the number of necrotic cells compared to a hold temperature of 4° C. (FIG. 28).

Combined Effects of Time, Temperature and Solution Conditions on Nutrient Uptake by Endocytosis

In order to monitor and quantify nutrient uptake by endocytosis, autografts were harvested and bone marrow stromal cells (BMSCs) were isolated using standard protocols. Prior to testing, BMSCs were removed from media, washed, then treated with ART352-L (0.8 ng/μL). Liposomes were tagged with the lipophilic fluorescent dye, DiI, in order to track their distribution.

BMSCs treated with ART352-L were then held at either 23° C. or at 37° C. for 15-120 minutes, e.g., the proposed duration of the ex vivo incubation step. These data demonstrate:
The uptake of fluorescent-labeled liposomes, i.e., ART352-L, increases as a function of time (FIG. 29).
The rate of uptake of the fluorescent-labeled liposomes increases as a function of temperature; e.g., at 23° C. the slope of the line=1142.3 and at 37° C. the slope of the line is =2792.9 (FIG. 29).

These data suggest that for the intended duration of the ex vivo hold period, incubation at 37° C. supports nutrient uptake by endocytosis better than an incubation temperature of 23° C. The next experiments tested whether the drug product ART352-L was stable for the intended duration of the ex vivo hold period if the incubation temperature was set at 37° C.

Stability of the Liposomal Wnt3a Polypeptide ART352-L as a Function of Time and Temperature

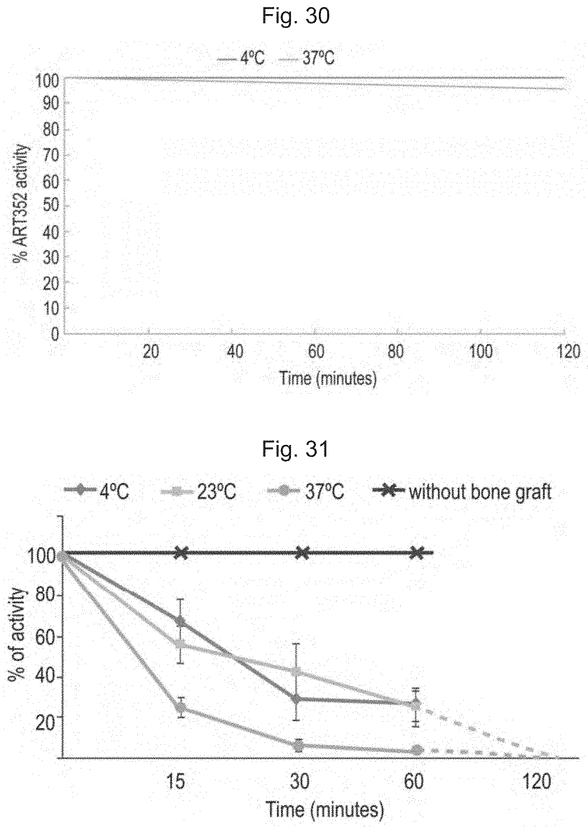

The stability of ART352-L was evaluated at 37° C. for the relevant time course of an ex vivo incubation step e.g., from 15 minutes to 2 hours. The stability of ART352-L at 4° C. was used as a positive control. Results from the stability evaluation indicate that ART352-L exhibits no detectable change in activity when it is maintained at 4° C. for 2h, as determined by regression analyses from stability studies conducted with non-GLP ART352-L (FIG. 30).

ART352-L exhibits a 4.9% change in activity when it is maintained at 37° C. for 2h (FIG. 30). Therefore, over the intended duration of the ex vivo incubation step e.g., 15 minutes to 2 hours, ART352-L shows minimal loss of activity. These data support development an autograft handling procedure in which a hold temperature of 37° C. is used.

Study to Evaluate the Rate of Active ART352-L Removal by Endocytosis from the Incubation Solution

ART352-L is endocytosed by cells in an autograft and as a consequence, ART352-L activity is lost from the incubation solution. The rate of decrease in concentration of ART352-L from the ex vivo incubation solution was monitored as a function of time and temperature.

Aliquots of the autograft were incubated in ART352-L at the indicated temperatures for the indicated time periods. At the conclusion of the time period, the aliquot of autograft was removed from the incubation solution, and an LSL assay was used to detect active ART352-L remaining in the incubation solution.

In cases where no bone graft is included, 100% of the initial ART352-L activity remains in the incubation solution (FIG. 31). In cases where an autograft is included in the incubation solution, after a 15-minute incubation at 4° C., a majority (68%) of the initial ART352-L activity remains in the incubation solution (FIG. 31). After a 15-minute incubation at 23° C., 56% of the initial ART352-L activity remains in the incubation solution (FIG. 31).

After a 15-minute incubation at the intended target temperature of 37° C., 24% of the initial ART352-L activity remains in the incubation solution (FIG. 31). After 30 minutes of incubation, 6% of the original ART352-L activity remains (FIG. 31). After 60 minutes the amount of active ART352-L remaining in the incubation solution is 2% (FIG. 31).

Assessment of ART352-L Treated Autografts

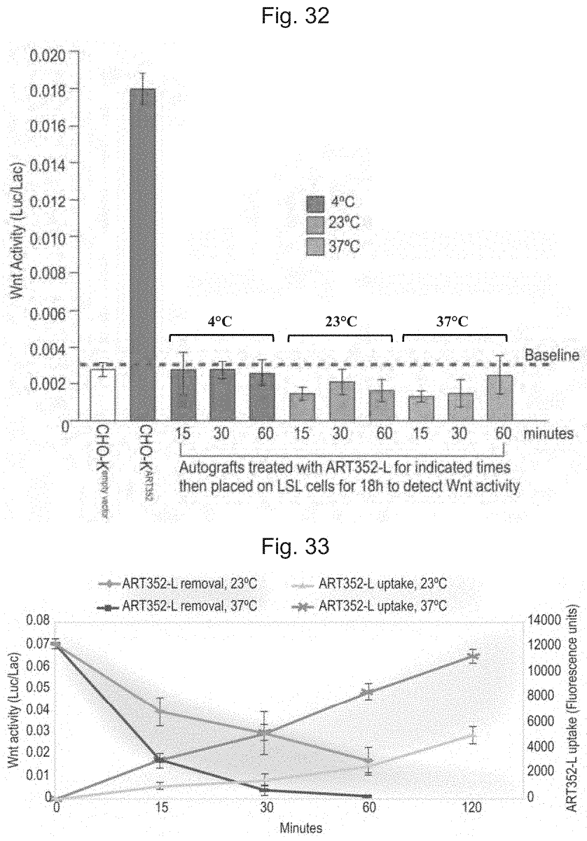

LSL cell-based assay was utilized to detect whether residual, free, active ART352-L was associated with the ART352-L treated autografts. A positive and a negative control were used in this series of experiments: the negative control consisted of CHO-K1 line carrying an empty expression vector (FIG. 32). The positive control consisted of the same CHO-K1 line carrying an ART352 expression vector (FIG. 32). The level of Wnt activity detected in CHO-K1 empty vector cells was established as baseline (FIG. 32).

Autografts were harvested from adult rats, treated with ART352-L (effective concentration=0.86 μg/mL) then incubated for 15, 30, or 60 minutes at 4° C. (blue bars), 23° C. (green bars), or 37° C. (FIG. 32). After the indicated ex vivo hold period, ART352-L autografts were placed on LSL cells and incubated for 18h, after which luciferase expression levels were quantified.

As shown in FIG. 32, no residual, free, active ART352-L was found to be associated with any ART352-L treated autografts.

Time- and Temperature-Dependent Removal of Active ART352-L from the Incubation Solution and its Endocytic Uptake by Cells Derived from the Autograft

Two quantitative analyses were performed to assess the rate of removal of residual, free, active ART532-L from the incubation solution, and to assess the rate of ART352-L endocytosis by cells in an autograft.
To measure removal of free, active ART352-L from the incubation solution, autografts were harvested and immediately placed in incubation solution containing ART352-L (effective concentration=0.86 ng/μL). The autografts in their incubation solutions were held at either 23° C. or 37° C. After 15, 30, and 60 minutes aliquots of the various incubation solutions were removed and tested for ART352-L activity using the LSL assay.
To assess the rate of ART352-L endocytosis by cells in an autograft, autografts were harvested and BMSCs were isolated following a standardized procedure. Cell number was standardized then wells were treated with ART352-L that was tagged with a fluorescent lipophilic dye, DiI. Cells were held at either 23° C. or 37° C. After 15, 30, 60, and 120 minutes, cells were pelleted, suspended in PBS then fluorescent signal was quantified using a plate reader

These data demonstrated that the movement of ART352-L from the incubation solution into cells of the autograft as a function of time and temperature (FIG. 33).

### Example 10

The following table 7 illustrates sequences disclosed in this application.

| | | SEQ ID |
|---|---|---|
| Protein Name | | NO: |
| Wnt3A | MAPLGYFLLLCSLKQALGSYPIWWSLAVGPQYSSLGSQPILCASIP | 1 |
| polypeptide | GLVPKQLRFCRNYVEIMPSVAEGIKIGIQECQHQFRGRRWNCTTV | |
| (isoform 2) | HDSLAIFGPVLDKATRESAFVHAIASAGVAFAVTRSCAEGTAAIC | |
| (Homo sapiens) | GCSSRHQGSPGKGWKWGGCSEDIEFGGMVSREFADARENRPDAR | |
| | SAMNRHNNEAGRQAIASHMHLKCKCHGLSGSCEVKTCWWSQPD | |
| | FRAIGDFLKDKYDSASEMVVEKHRESRGWVETLRPRYTYFKVPT | |
| | ERDLVYYEASPNFCEPNPETGSFGTRDRTCNVSSHGIDGCDLLCC | |
| | GRGHNARAERRREKCRCVFHWCCYVSCQECTRVYDVHTCKNPG | |
| | SRAGNSAHQPPHPQPPVRFHPPLRRAGKVP | |
| Wnt3A | MAPLGYFLLLCSLKQALGSYPIWWSLAVGPQYSSLGSQPILCASIP | 2 |
| polypeptide | GLVPKQLRFCRNYVEIMPSVAEGIKIGIQECQHQFRGRRWNCTTV | |
| (Isoform 1) | HDSLAIFGPVLDKATRESAFVHAIASAGVAFAVTRSCAEGTAAIC | |
| (Homo sapiens) | GCSSRHQGSPGKGWKWGGCSEDIEFGGMVSREFADARENRPDAR | |
| | SAMNRHNNEAGRQAIASHMHLKCKCHGLSGSCEVKTCWWSQPD | |
| | FRAIGDFLKDKYDSASEMVVEKHRESRGWVETLRPRYTYFKVPT | |
| | ERDLVYYEASPNFCEPNPETGSFGTRDRTCNVSSHGIDGCDLLCC | |
| | GRGHNARAERRREKCRCVFHWCCYVSCQECTRVYDVHTCK | |
| Wnt3A | ATGGCCCCAC TCGGATACTT CTTACTCCTC TGCAGCCTGA | 3 |
| nucleotide | AGCAGGCTCT GGGCAGCTAC CCGATCTGGT GGTCGCTGGC | |
| (Homo sapiens) | TGTTGGGCCA CAGTATTCCT CCCTGGGCTC GCAGCCCATC | |
| | CTGTGTGCCA GCATCCCGGG CCTGGTCCCC AAGCAGCTCC | |
| | GCTTCTGCAG GAACTACGTG GAGATCATGC CCAGCGTGGC | |
| | CGAGGGCATC AAGATTGGCA TCCAGGAGTG CCAGCACCAG | |
| | TTCCGCGGCC GCCGGTGGAA CTGCACCACC GTCCACGACA | |
| | GCCTGGCCAT CTTCGGGCCC GTGCTGGACA AAGCTACCAG | |
| | GGAGTCGGCC TTTGTCCACG CCATTGCCTC AGCCGGTGTG | |
| | GCCTTTGCAG TGACACGCTC ATGTGCAGAA GGCACGGCCG | |
| | CCATCTGTGG CTGCAGCAGC CGCCACCAGG GCTCACCAGG | |
| | CAAGGGCTGG AAGTGGGGTG GCTGTAGCGA GGACATCGAG | |
| | TTTGGTGGGA TGGTGTCTCG GGAGTTCGCC GACGCCCGGG | |
| | AGAACCGGCC AGATGCCCGC TCAGCCATGA ACCGCCACAA | |
| | CAACGAGGCT GGGCGCCAGG CCATCGCCAG CCACATGCAC | |
| | CTCAAGTGCA AGTGCCACGG GCTGTCGGGC AGCTGCGAGG | |
| | TGAAGACATG CTGGTGGTCG CAACCCGACT TCCGCGCCAT | |
| | CGGTGACTTC CTCAAGGACA AGTACGACAG CGCCTCGGAG | |
| | ATGGTGGTGG AGAAGCACCG GGAGTCCCGC GGCTGGGTGG | |
| | AGACCCTGCG GCCGCGCTAC ACCTACTTCA AGGTGCCCAC | |
| | GGAGCGCGAC CTGGTCTACT ACGAGGCCTC GCCCAACTTC | |
| | TGCGAGCCCA ACCCTGAGAC GGGCTCCTTC GGCACGCGCG | |
| | ACCGCACCTG CAACGTCAGC TCGCACGGCA TCGACGGCTG | |
| | CGACCTGCTG TGCTGCGGCC GCGGCCACAA CGCGCGAGCG | |
| | GAGCGGCGCC GGGAGAAGTG CCGCTGCGTG TTCCACTGGT | |
| | GCTGCTACGT CAGCTGCCAG GAGTGCACGC GCGTCTACGA | |
| | CGTGCACACC TGCAAGTAGG CACCGGCCGC GGCTCCCCCT | |
| | GGACGGGGCG GGCCCTGCCT GAGGGTGGGC TTTTCCCTGG | |
| | GTGGAGCAGG ACTCCCACCT AAACGGGGCA GTACTCCTCC | |
| | CTGGGGGCGG GACTCCTCCC TGGGGGTGGG GCTCCTACCT | |
| | GGGGGCAGAA CTCCTACCTG AAGGCAGGGC TCCTCCCTGG | |
| | AGCTAGTGTC TCCTCTCTGG TGGCTGGGCT GCTCCTGAAT | |
| | GAGGCGGAGC TCCAGGATGG GGAGGGGCTC TGCGTTGGCT | |
| | TCTCCCTGGG GACGGGGCTC CCCTGGACAG AGGCGGGGCT | |
| | ACAGATTGGG CGGGGCTTCT CTTGGGTGGG ACAGGGCTTC | |
| | TCCTGCGGGG GCGAGGCCCC TCCCAGTAAG GGCGTGGCTC | |
| | TGGGTGGGCG GGGCACTAGG TAGGCTTCTA CCTGCAGGCG | |
| | GGGCTCCTCC TGAAGGAGGC GGGGCTCTAG GATGGGGCAC | |
| | GGCTCTGGGG TAGGCTGCTC CCTGAGGGCG GAGCGCCTCC | |
| | TTAGGAGTGG GGTTTTATGG TGGATGAGGC TTCTTCCTGG | |
| | ATGGGGCAGA GCTTCTCCTG ACCAGGGCAA GGCCCCTTCC | |
| | ACGGGGGCTG TGGCTCTGGG TGGGCGTGGC CTGCATAGGC | |
| | TCCTTCCTGT GGGTGGGGCT TCTCTGGGAC CAGGCTCCAA | |
| | TGGGGCGGGG CTTCTCTCCG CGGGTGGGAC TCTTCCCTGG | |
| | GAACCGCCCT CCTGATTAAG GCGTGGCTTC TGCAGGAATC | |
| | CCGGCTCCAG AGCAGGAAAT TCAGCCCACC AGCCACCTCA | |
| | TCCCCAACCC CCTGTAAGGT TCCATCCACC CCTGCGTCGA | |
| | GCTGGGAAGG TTCCATGAAG CGAGTCGGGT CCCCAACCCG | |
| | TGCCCCTGGG ATCCGAGGGC CCCTCTCCAA GCGCCTGGCT | |
| | TTGGAATGCT CCAGGCGCGC CGACGCCTGT GCCACCCCTT | |
| | CCTCAGCCTG GGGTTTGACC ACCCACCTGA CCAGGGGCCC | |
| | TACCTGGGGA AAGCCTGAAG GGCCTCCCAG CCCCCAACCC | |
| | CAAGACCAAG CTTAGTCCTG GGAGAGGACA GGGACTTCGC | |
| | AGAGGCAAGC GACCGAGGCC CTCCCAAAGA GGCCCGCCCT | |
| | GCCCGGGCTC CCACACCGTC AGGTACTCCT GCCAGGGAAC | |
| | TGGCCTGCTG CGCCCCAGGC CCCGCCCGTC TCTGCTCTGC | |
| | TCAGCTGCGC CCCCTTCTTT GCAGCTGCCC AGCCCCTCCT | |
| | CCCTGCCCTC GGGTCTCCCC ACCTGCACTC CATCCAGCTA | |
| | CAGGAGAGAT AGAAGCCTCT CGTCCCGTCC CTCCCTTTCC | |
| | TCCGCCTGTC CACAGCCCCT TAAGGGAAAG GTAGGAAGAG | |
| | AGGTCCAGCC CCCCAGGCTG CCCAGAGCTG CTGGTCTCAT | |
| | TTGGGGGCGT TCGGGAGGTT TGGGGGGCAT CAACCCCCCG | |
| | ACTGTGCTGC TCGCGAAGGT CCCACAGCCC TGAGATGGGC | |
| | CGGCCCCCTT CCTGGCCCCT CATGGCGGGA CTGGAGAAAT | |
| | GGTCCGCTTT CCTGGAGCCA ATGGCCCGGC CCCTCCTGAC | |
| | TCATCCGCCT GGCCCGGGAA TGAATGGGGA GGCCGCTGAA | |
| | CCCACCCGGC CCATATCCCT GGTTGCCTCA TGGCCAGCGC | |
| | CCCTCAGCCT CTGCCACTGT GAACCGGCTC CCACCCTCAA | |
| | GGTGCGGGGA GAAGAAGCGG CCAGGCGGGG CGCCCCAAGA | |
| | GCCCAAAAGA GGGCACACCG CCATCCTCTG CCTCAAATTC | |
| | TGCGTTTTTG GTTTTAATGT TATATC | |
| Frizzled-8 | MEWGYLLEVTSLLAALALLQRSSGAAAASAKELACQEITVPLCK | 4 |
| (precursor) | GIGYNYTYMPNQFNHDTQDEAGLEVHQFWPLVEIQCSPDLKFFLC | |
| (Homo sapiens) | SMYTPICLEDYKKPLPPCRSVCERAKAGCAPLMRQYGFAWPDRM | |
| (NCBI Accession | RCDRLPEQGNPDTLCMDYNRTDLTTAAPSPPRRLPPPPPGEQPPSG | |
| No. | SGHGRPPGARPPHRGGGRGGGGGDAAAPPARGGGGGGKARPPG | |
| NP_114072.1) | GGAAPCEPGCQCRAPMVSVSSERHPLYNRVKTGQIANCALPCHN | |
| | PFFSQDERAFTVFWIGLWSVLCFVSTFATVSTFLIDMERFKYPERPI | |
| | IFLSACYLFVSVGYLVRLVAGHEKVACSGGAPGAGGAGGAGGAA | |
| | GA AGAAGAGAGGPGGRGEYEELGAVEQHVRYETTGPALCTVVF | |
| | LLVYFFGMASSIWWVILSLTWFLAAGMKWGNEAIAGYSQYFHLA | |
| | AWLVPSVKSIAVLALSSVDGDPVAGICYVGNQSLDNLRGFVLAPL | |
| | VIYLFIGTMFLLAGFVSLFRIRSVIKQQDGPTKTHKLEKLMIRLGLF | |
| | TVLYTVPAAVVVACLFYEQHNRPRWEATHNCPCLRDLQPDQAR | |
| | RPDYAVFMLKYFMCLVVGITSGVWVWSGKTLESWRSLCTRCCW | |
| | ASKGAAVGGGAGATAAGGGGGPGGGGGGGPGGGGGPGGGGGS | |
| | LYSDVSTGLTWRSGTASSVSYPKQMPLSQV | |
| Frizzled-8 | MEWGYLLEVTSLLAALALLQRSSGAAAASAKELACQEITVPLCK | 5 |
| fusion | GIGYNYTYMPNQFNHDTQDEAGLEVHQFWPLVEIQCSPDLKFFLC | |
| protein 1 | SMYTPICLEDYKKPLPPCRSVCERAKAGCAPLMRQYGFAWPDRM | |
| | CRDRLPEQGNPDTLCMDYGGGGGGGDKTHTCPPCPAPELLGGPS | |
| | VFLFPPKPKDTLMISRTPEVTCVVVDVSHEDPEVKFNWYVDGVE | |
| | VHNAKTKPREEQYNSTYRVVSVLTVLHQDWLNGKEYKCKVSNK | |
| | ALPAPIEKTISKAKGQPREPQVYTLPPSRDELTKNQVSLTCLVKGF | |
| | YPSDIAVEWESNGQPENNYKTTPPVLDSDGSFFLYSKLTVDKSRW | |
| | QQGNVFSCSVMHEALHNHYTQKSLSLSPGK | |
| Wntless | MAGAIIENMSTKKLCIVGGILLVFQIIAFLVGGLIAPGPTTAVSYMS | 7 |
| (precursor) | VKCVDARKNHHKTKWFVPWGPNHCDKIRDIEEAIPREIEANDIVF | |
| (Homo sapiens) | SVHIPLPHMEMSPWFQFMLFILQLDIAFKLNNQIRENAEVSMDVSL | |
| (NCBI Accession | AYRDDAFAEWTEMAHERVPRKLKCTFTSPKTPEHEGRYYECDVL | |
| No. Q5T9L3.2) | PFMEIGSVAHKFYLLNIRLPVNEKKKINVGIGEIKDIRLVGIHQNGG | |
| | FTKVWFAMKTFLTPSIFIIMVWYWRRITMMSRPPVLLEKVIFALGI | |
| | SMTFINIPVEWFSIGFDWTWMLLFGDIRQGIFYAMLLSFWIIFCGE | |
| | HMMDQHERNHIAGYWKQVGPIAVGSFCLFIFDMCERGVQLTNPF | |
| | YSIWTTDIGTELAMAFIIVAGICLCLYFLFLCFMVFQVFRNISGKQS | |
| | SLPAMSKVRRLHYEGLIFRFKFLMLITLACAAMTVIFFIVSQVTEG | |
| | HWKWGGVTVQVNSAFFTGIYGMWNLYVFALMFLYAPSHKNYG | |
| | EDQSNGDLGVHSGEELQLTTTITHVDGPTEIYKLTRKEAQE | |
| Afamin | MKLLKLTGFIFFLFFLTESLTLPTQPRDIENFNSTQKFIEDNIEYITII | 8 |
| (Homo sapiens) | AFAQYVQEATFEEMEKLVKDMVEYKDRCMADKTLPECSKLPNN | |
| (NCBI Accession | VLQEKICAMEGLPQKHNFSHCCSKVDAQRRLCFFYNKKSDVGFL | |
| No. | PPFPTLDPEEKCQAYESNRESLLNHFLYEVARRNPFVFAPTLLTVA | |
| AAA21612.1) | VHFEEVAKSCCEEQNKVNCLQTRAIPVTQYLKAFSSYQKHVCGA | |
| | LLKFGTKVVHFIYIAILSQKFPKIEFKELISLVEDVSSNYDGCCEGD | |
| | VVQCIRDTSKVMNHICSKQDSISSKIKECCEKKIPERGQCIINSNKD | |
| | DRPKDLSLREGKFTDSENVCQERDADPDTFFAKFTFEYSRRHPDL | |
| | SIPELLRIVQIYKDLLRNCCNTENPPGCYRYAEDKFNETTEKSLKM | |
| | VQQECKHFQNLGKDGLKYHYLIRLTKIAPQLSTEELVSLGEKMVT | |
| | AFTTCCTLSEEFACVDNLADLVFGELCGVNENRTINPAVDHCCKT | |
| | NFAFRRPCFESLKADKTYVPPPFSQDLFTFHADMCQSQNEELQRK | |
| | TDRFLVNLVKLKHELTDEELQSLFTNFANVVDKCCKAESPEVCFN | |
| | EESPKIGN | |
| Porcupine | MATFSRQEFFQQLLQGCLLPTAQQGLDQIWLLLAICLACRLLWRL | 9 |
| (Homo sapiens) | GLPSYLKHASTVAGGFFSLYHFFQLHMVWVVLLSLLCYLVLFLC | |
| (NCBI Accession | RHSSHRGVFLSVTILIYLLMGEMHMVDTVTWHKMRGAQMIVAM | |
| No. | KAVSLGFDLDRGEVGTVPSPVEFMGYLYFVGTIVFGPWISFHSYL | |
| NP_073736.2) | QAVQGRPLSCRWLQKVARSLALALLCLVLSTCVGPYLFPYFIPLN | |
| (isoform A) | GDRLLRKWLRAYESAVSFHFSNYFVGFLSEATATLAGAGFTEEK | |
| | DHLEWDLTVSKPLNVELPRSMVEVVTSWNLPMSYWLNNYVFKN | |
| | ALRLGTFSAVLVTYAASALLHGFSFHLAAVLLSLAFITYVEHVLR | |
| | KRLARILSACVLSKRCPPDCSHQHRLGLGVRALNLLFGALAIFHL | |
| | AYLGSLFDVDVDDTTEEQGYGMAYTVHKWSELSWASHWVTFG | |
| | CWIFYRLIG | |
| Wnt3A Variant 1 | MEWSWVFLFFLSVTTGVHSGVAMPGAEDDVVRENLYFQGKDGS | 10 |
| (SP-PA-TEV- | SYPIWWSLAVGPQYSSLGSQPILCASIPGLVPKQLRFCRNYVEIMP | |
| hWnt3A) | SVAEGIKIGIQECQHQFRGRRWNCTTVHDSLAIFGPVLDKATRESA | |
| Signal Peptide | FVHAIASAGVAFAVTRSCAEGTAAICGCSSRHQGSPGKGWKWGG | |
| (SP)-VH21 | CSEDIEFGGMVSREFADARENRPDARSAMNRHNNEAGRQAIASH | |
| | MHLKCKCHGLSGSCEVKTCWWSQPDFRAIGDFLKDKYDSASEM | |
| | VVEKHRESRGWVETLRPRYTYFKVPTERDLVYYEASPNFCEPNPE | |
| | TGSFGTRDRTCNVSSHGIDGCDLLCCGRGHNARAERRREKCRCV | |
| | FHWCCYVSCQECTRVYDVHTCK | |
| Wnt3A Variant2 | MEWSWVFLFFLSVTTGVHSDYKDDDDKENLYFQGSYPIWWSLA | 11 |
| (SP-FLAG-TEV)- | VGPQYSSLGSQPILCASIPGLVPKQLRFCRNYVEIMPSVAEGIKIGI | |
| hWnt3A | QECQHQFRGRRWNCTTVHDSLAIFGPVLDKATRESAFVHAIASAG | |
| SP-VH21) | VAFAVTRSCAEGTAAICGCSSRHQGSPGKGWKWGGCSEDIEFGG | |
| | MVSREFADARENRPDARSAMNRHNNEAGRQAIASHMHLKCKCH | |
| | GLSGSCEVKTCWWSQPDFRAIGDFLKDKYDSASEMVVEKHRESR | |
| | GWVETLRPRYTYFKVPTERDLVYYEASPNFCEPNPETGSFGTRDR | |
| | TCNVSSHGIDGCDLLCCGRGHNARAERRREKCRCVFHWCCYVSC | |
| | QECTRVYDVHTCK | |
| Wnt3A Variant 3 | MEWSWVFLFFLSVTTGVHSHHHHHHENLYFQGSYPIWWSLAVG | 12 |
| (SP-His₆- | PQYSSLGSQPILCASIPGLVPKQLRFCRNYVEIMPSVAEGIKIGIQEC | |
| TEV-hWnt3A) | QHQFRGRRWNCTTVHDSLAIFGPVLDKATRESAFVHAIASAGVAF | |
| (SP-VH21) | AVTRSCAEGTAAICGCSSRHQGSPGKGWKWGGCSEDIEFGGMVS | |
| | REFADARENRPDARSAMNRHNNEAGRQAIASHMHLKCKCHGLS | |
| | GSCEVKTCWWSQPDFRAIGDFLKDKYDSASEMVVEKHRESRGW | |
| | VETLRPRYTYFKVPTERDLVYYEASPNFCEPNPETGSFGTRDRTC | |
| | NVSSHGIDGCDLLCCGRGHNARAERRREKCRCVFHWCCYVSCQE | |
| | CTRVYDVHTCK | |
| Wnt3A Variant 4 | MEWSWVFLFFLSVTTGVHSHHHHHHSYPIWWSLAVGPQYSSLGS | 13 |
| (His-hWnt3A) | QPILCASIPGLVPKQLRFCRNYVEIMPSVAEGIKIGIQECQHQFRGR | |
| | RWNCTTVHDSLAIFGPVLDKATRESAFVHAIASAGVAFAVTRSCA | |
| | EGTAAICGCSSRHQGSPGKGWKWGGCSEDIEFGGMVSREFADAR | |
| | ENRPDARSAMNRHNNEAGRQAIASHMHLKCKCHGLSGSCEVKT | |
| | CWWSQPDFRAIGDFLKDKYDSASEMVVEKHRESRGWVETLRPR | |
| | YTYFKVPTERDLVYYEASPNFCEPNPETGSFGTRDRTCNVSSHGID | |
| | GCDLLCCGRGHNARAERRREKCRCVFHWCCYVSCQECTRVYDV | |
| | HTCK | |
| Wnt3A Variant 5 | MEWSWVFLFFLSVTTGVHSHHHHHHGGGSYPIWWSLAVGPQYS | 14 |
| (His-GGG- | SLGSQPILCASIPGLVPKQLRFCRNYVEIMPSVAEGIKIGIQECQHQ | |
| hWnt3A) | FRGRRWNCTTVHDSLAIFGPVLDKATRESAFVHAIASAGVAFAVT | |
| | RSCAEGTAAICGCSSRHQGSPGKGWKWGGCSEDIEFGGMVSREF | |
| | ADARENRPDARSAMNRHNNEAGRQAIASHMHLKCKCHGLSGSC | |
| | EVKTCWWSQPDFRAIGDFLKDKYDSASEMVVEKHRESRGWVET | |
| | LRPRYTYFKVPTERDLVYYEASPNFCEPNPETGSFGTRDRTCNVSS | |
| | HGIDGCDLLCCGRGHNARAERRREKCRCVFHWCCYVSCQECTR | |
| | VYDVHTCK | |
| Wnt3A Variant 5 | MEWSWVFLFFLSVTTGVHSHHHHHHGGGGAGGGGSYPIWWSLA | 15 |
| (His-G4AG4- | VGPQYSSLGSQPILCASIPGLVPKQLRFCRNYVEIMPSVAEGIKIGI | |
| hWnt3A) | QECQHQFRGRRWNCTTVHDSLAIFGPVLDKATRESAFVHAIASAG | |
| | VAFAVTRSCAEGTAAICGCSSRHQGSPGKGWKWGGCSEDIEFGG | |
| | MVSREFADARENRPDARSAMNRHNNEAGRQAIASHMHLKCKCH | |
| | GLSGSCEVKTCWWSQPDFRAIGDFLKDKYDSASEMVVEKHRESR | |
| | GWVETLRPRYTYFKVPTERDLVYYEASPNFCEPNPETGSFGTRDR | |
| | TCNVSSHGIDGCDLLCCGRGHNARAERRREKCRCVFHWCCYVSC | |
| | QECTRVYDVHTCK | |
| Frizzled-8 | MEWGYLLEVTSLLAALALLQRSSGAAAASAKELACQEITVPLCK | 16 |
| (FZD8) truncated | GIGYNYTYMPNQFNHDTQDEAGLEVHQFWPLVEIQCSPDLKFFLC | |
| variant 1 | SMYTPICLEDYKKPLPPCRSVCERAKAGCAPLMRQYGFAWPDRM | |
| | RCDRLPEQGNPDTLCMDY | |
| Frizz1ed-8 | MEWGYLLEVTSLLAALALLQRSSGAAAASAKELACQEITVPLCK | 17 |
| (FZD8) truncated | GIGYNYTYMPNQFNHDTQDEAGLEVHQFWPLVEIQCSPDLKFFLC | |
| variant 2 | SMYTPICLEDYKKPLPPCRSVCERAKAGCAPLMRQYGFAWPDRM | |
| | RCDRLPEQGNPDTLCMDYNRTDLTTAAPSPPRRLPPPPP | |
| Frizzled-8 fusion | MEWGYLLEVTSLLAALALLQRSSGAAAASAKELACQEITVPLCK | 18 |
| protein 2 | GIGYNYTYMPNQFNHDTQDEAGLEVHQFWPLVEIQCSPDLKFFLC | |
| | SMYTPICLEDYKKPLPPCRSVCERAKAGCAPLMRQYGFAWPDRM | |
| | RCDRLPEQGNPDTLCMDYNRTDLTTAAPSPPRRLPPPPPGGGGGG | |
| | GDKTHTCPPCPAPELLGGPSVFLFPPKPKDTLMISRTPEVTCVVVD | |
| | VSHEDPEVKFNWYVDGVEVHNAKTKPREEQYNSTYRVVSVLTV | |
| | LHQDWLNGKEYKCKVSNKALPAPIEKTISKAKGQPREPQVYTLPP | |
| | SRDELTKNQVSLTCLVKGFYPSDIAVEWESNGQPENNYKTTPPVL | |
| | DSDGSFFLYSKLTVDKSRWQQGNVFSCSVMHEALHNHYTQKSLS | |
| | LSPGK | |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of preparing a functionally active Wnt polypeptide, comprising:
(a) co-expressing a Wnt polypeptide and a chaperone in a cell in a conditioned media to generate a plurality of Wnt polypeptide-chaperone complexes;
(b) harvesting the plurality of Wnt polypeptide-chaperone complexes from the conditioned media;
(c) incubating the plurality of Wnt polypeptide-chaperone complexes with a buffer comprising a sugar detergent to generate a mixture comprising a first Wnt composition comprising a functionally inactive Wnt polypeptide and a chaperone composition;
(d) separating the first Wnt composition from the mixture with a column immobilized with a sulfonated polyaromatic compound to generate a second Wnt composition comprising the functionally active Wnt polypeptide and the sugar detergent; and
(e) contacting the second Wnt composition with an aqueous solution of liposomes to generate a final Wnt composition comprising a functionally active Wnt polypeptide.

2-21. (canceled)

22. The method of claim 1, wherein the chaperone comprises a Frizzled protein.

23. The method of claim 1, wherein the chaperone comprises a Frizzled-8 fusion protein.

24. The method of claim 23, wherein the Frizzled-8 fusion protein comprises a truncated Frizzled-8 protein, and wherein the truncated Frizzled-8 protein comprises a cysteine-rich region (CRD) of Frizzled-8.

25. (canceled)

26. (canceled)

27. The method of claim 23, wherein the Frizzled-8 fusion protein further comprises an IgG Fc portion.

28-30. (canceled)

31. The method of claim 1, wherein the Wnt polypeptide is a Wnt3A polypeptide.

32. The method of claim 31, wherein the Wnt3A polypeptide comprises about 90%, 95%, 99%, or more sequence identity to SEQ ID NO: 1.

33. The method of claim 31, wherein the Wnt3A polypeptide comprises a truncation of about 1 to about 33 amino acids, optionally a C-terminal truncation.

34. (canceled)

35. The method of claim 1, wherein the Wnt polypeptide comprises a lipid modification at an amino acid position corresponding to amino acid residue 209 as set forth in SEQ ID NO: 1.

36-100. (canceled)

101. A mammalian cell comprising an exogenous Wnt polypeptide and an exogenous chaperone protein.

102. The mammalian cell of claim 101, wherein the chaperone comprises a Frizzled protein.

103. The mammalian cell of claim 102, wherein the chaperone comprises a Frizzled-8 fusion protein.

104. The mammalian cell of claim 103, wherein the Frizzled-8 fusion protein comprises a truncated Frizzled-8 protein, wherein the truncated Frizzled-8 protein comprises a cysteine-rich region (CRD) of Frizzled-8.

105. The mammalian cell of claim 101, wherein the Wnt polypeptide is a Wnt3A polypeptide.

106. The mammalian cell of claim 105, wherein the Wnt3A polypeptide comprises at least 90% sequence identity to SEQ ID NO: 1.

107. The mammalian cell of claim 105, wherein the Wnt3A polypeptide comprises a truncation of about 1 to about 33 amino acids, optionally a C-terminal truncation.

108. A pharmaceutical composition, wherein the pharmaceutical composition comprises a liposomal Wnt3A polypeptide, wherein the amount of active Wnt3A polypeptide present in the pharmaceutical composition provides for an EC50 of about equal to or less than 0.5 ng/uL when administered to a bone graft material.

109. The pharmaceutical composition of claim 108, wherein the Wnt3A polypeptide comprises at least 90% sequence identity to SEQ ID NO: 1.

110. The pharmaceutical composition of claim 108, wherein the Wnt3A polypeptide comprises a truncation of about 1 to about 33 amino acids, optionally a C-terminal truncation.

111. The pharmaceutical composition of claim 108, wherein the Wnt polypeptide comprises a lipid modification at an amino acid position corresponding to amino acid residue 209 as set forth in SEQ ID NO: 1.


SEQUENCE LISTING (33 sequences)

SEQ ID NO: 1 - protein, 385 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCKNPGSRAGNSAHQPPHPQPPVRFHPPLRRAGKVP

SEQ ID NO: 2 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 3 - DNA, 2826 bp, Homo sapiens
atggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcggagcgcctccttaggagtggggttttatggtggatgaggcttcttcctggatggggcagagcttctcctgaccagggcaaggccccttccacgggggctgtggctctgggtgggcgtggcctgcataggctccttcctgtgggtggggcttctctgggaccaggctccaatggggcggggcttctctccgcgggtgggactcttccctgggaaccgccctcctgattaaggcgtggcttctgcaggaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaagggcctcccagcccccaaccccaagaccaagcttagtcctgggagaggacagggacttcgcagaggcaagcgaccgaggccctcccaaagaggcccgccctgcccgggctcccacaccgtcaggtactcctgccagggaactggcctgctgcgccccaggccccgcccgtctctgctctgctcagctgcgcccccttctttgcagctgcccagcccctcctccctgccctcgggtctccccacctgcactccatccagctacaggagagatagaagcctctcgtcccgtccctccctttcctccgcctgtccacagccccttaagggaaaggtaggaagagaggtccagccccccaggctgcccagagctgctggtctcatttgggggcgttcgggaggtttggggggcatcaaccccccgactgtgctgctcgcgaaggtcccacagccctgagatgggccggcccccttcctggcccctcatggcgggactggagaaatggtccgctttcctggagccaatggcccggcccctcctgactcatccgcctggcccgggaatgaatggggaggccgctgaacccacccggcccatatccctggttgcctcatggccagcgcccctcagcctctgccactgtgaaccggctcccaccctcaaggtgcggggagaagaagcggccaggcggggcgccccaagagcccaaaagagggcacaccgccatcctctgcctcaaattctgcgtttttggttttaatgttatatc

SEQ ID NO: 4 - protein, 694 aa, Homo sapiens
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPGEQPPSGSGHGRPPGARPPHRGGGRGGGGGDAAAPPARGGGGGGKARPPGGGAAPCEPGCQCRAPMVSVSSERHPLYNRVKTGQIANCALPCHNPFFSQDERAFTVFWIGLWSVLCFVSTFATVSTFLIDMERFKYPERPIIFLSACYLFVSVGYLVRLVAGHEKVACSGGAPGAGGAGGAGGAAAGAGAAGAGAGGPGGRGEYEELGAVEQHVRYETTGPALCTVVFLLVYFFGMASSIWWVILSLTWFLAAGMKWGNEAIAGYSQYFHLAAWLVPSVKSIAVLALSSVDGDPVAGICYVGNQSLDNLRGFVLAPLVIYLFIGTMFLLAGFVSLFRIRSVIKQQDGPTKTHKLEKLMIRLGLFTVLYTVPAAVVVACLFYEQHNRPRWEATHNCPCLRDLQPDQARRPDYAVFMLKYFMCLVVGITSGVWVWSGKTLESWRSLCTRCCWASKGAAVGGGAGATAAGGGGGPGGGGGGGPGGGGGPGGGGGSLYSDVSTGLTWRSGTASSVSYPKQMPLSQV

SEQ ID NO: 5 - protein, 385 aa, Artificial Sequence
  Description of Artificial Sequence Synthetic polypeptide
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 6 aa, Artificial Sequence
  Description of Artificial Sequence Synthetic peptide
IEGRMD

SEQ ID NO: 7 - protein, 541 aa, Homo sapiens
MAGAIIENMSTKKLCIVGGILLVFQIIAFLVGGLIAPGPTTAVSYMSVKCVDARKNHHKTKWFVPWGPNHCDKIRDIEEAIPREIEANDIVFSVHIPLPHMEMSPWFQFMLFILQLDIAFKLNNQIRENAEVSMDVSLAYRDDAFAEWTEMAHERVPRKLKCTFTSPKTPEHEGRYYECDVLPFMEIGSVAHKFYLLNIRLPVNEKKKINVGIGEIKDIRLVGIHQNGGFTKVWFAMKTFLTPSIFIIMVWYWRRITMMSRPPVLLEKVIFALGISMTFINIPVEWFSIGFDWTWMLLFGDIRQGIFYAMLLSFWIIFCGEHMMDQHERNHIAGYWKQVGPIAVGSFCLFIFDMCERGVQLTNPFYSIWTTDIGTELAMAFIIVAGICLCLYFLFLCFMVFQVFRNISGKQSSLPAMSKVRRLHYEGLIFRFKFLMLITLACAAMTVIFFIVSQVTEGHWKWGGVTVQVNSAFFTGIYGMWNLYVFALMFLYAPSHKNYGEDQSNGDLGVHSGEELQLTTTITHVDGPTEIYKLTRKEAQE

SEQ ID NO: 8 - protein, 599 aa, Homo sapiens
MKLLKLTGFIFFLFFLTESLTLPTQPRDIENFNSTQKFIEDNIEYITIIAFAQYVQEATFEEMEKLVKDMVEYKDRCMADKTLPECSKLPNNVLQEKICAMEGLPQKHNFSHCCSKVDAQRRLCFFYNKKSDVGFLPPFPTLDPEEKCQAYESNRESLLNHFLYEVARRNPFVFAPTLLTVAVHFEEVAKSCCEEQNKVNCLQTRAIPVTQYLKAFSSYQKHVCGALLKFGTKVVHFIYIAILSQKFPKIEFKELISLVEDVSSNYDGCCEGDVVQCIRDTSKVMNHICSKQDSISSKIKECCEKKIPERGQCIINSNKDDRPKDLSLREGKFTDSENVCQERDADPDTFFAKFTFEYSRRHPDLSIPELLRIVQIYKDLLRNCCNTENPPGCYRYAEDKFNETTEKSLKMVQQECKHFQNLGKDGLKYHYLIRLTKIAPQLSTEELVSLGEKMVTAFTTCCTLSEEFACVDNLADLVFGELCGVNENRTINPAVDHCCKTNFAFRRPCFESLKADKTYVPPPFSQDLFTFHADMCQSQNEELQRKTDRFLVNLVKLKHELTDEELQSLFTNFANVVDKCCKAESPEVCFNEESPKIGN

SEQ ID NO: 9 - protein, 450 aa, Homo sapiens
MATFSRQEFFQQLLQGCLLPTAQQGLDQIWLLLAICLACRLLWRLGLPSYLKHASTVAGGFFSLYHFFQLHMVWVVLLSLLCYLVLFLCRHSSHRGVFLSVTILIYLLMGEMHMVDTVTWHKMRGAQMIVAMKAVSLGFDLDRGEVGTVPSPVEFMGYLYFVGTIVFGPWISFHSYLQAVQGRPLSCRWLQKVARSLALALLCLVLSTCVGPYLFPYFIPLNGDRLLRKWLRAYESAVSFHFSNYFVGFLSEATATLAGAGFTEEKDHLEWDLTVSKPLNVELPRSMVEVVTSWNLPMSYWLNNYVFKNALRLGTFSAVLVTYAASALLHGFSFHLAAVLLSLAFITYVEHVLRKRLARILSACVLSKRCPPDCSHQHRLGLGVRALNLLFGALAIFHLAYLGSLFDVDVDDTTEEQGYGMAYTVHKWSELSWASHWVTFGCWIFYRLIG

SEQ ID NO: 10 - protein, 377 aa, Artificial Sequence
  Description of Artificial Sequence Synthetic polypeptide
MEWSWVFLFFLSVTTGVHSGVAMPGAEDDVVRENLYFQGKDGSSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 11 - protein, 368 aa, Artificial Sequence
  Description of Artificial Sequence Synthetic polypeptide
MEWSWVFLFFLSVTTGVHSDYKDDDDKENLYFQGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 12 - protein, 366 aa, Artificial Sequence
  Description of Artificial Sequence Synthetic polypeptide
MEWSWVFLFFLSVTTGVHSHHHHHHENLYFQGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 13 - protein, 359 aa, Artificial Sequence
  Description of Artificial Sequence Synthetic polypeptide
MEWSWVFLFFLSVTTGVHSHHHHHHSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 14 - protein, 362 aa, Artificial Sequence
  Description of Artificial Sequence Synthetic polypeptide
MEWSWVFLFFLSVTTGVHSHHHHHHGGGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 15 - protein, 368 aa, Artificial Sequence
  Description of Artificial Sequence Synthetic polypeptide
MEWSWVFLFFLSVTTGVHSHHHHHHGGGGAGGGGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 16 - protein, 151 aa, Homo sapiens
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDY

SEQ ID NO: 17 - protein, 172 aa, Homo sapiens
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPP

SEQ ID NO: 18 - protein, 406 aa, Artificial Sequence
  Description of Artificial Sequence Synthetic polypeptide
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 6 aa, Artificial Sequence
  Description of Artificial Sequence Synthetic 6xHis tag
HHHHHH

SEQ ID NO: 20 - protein, 10 aa, Artificial Sequence
  Description of Artificial Sequence Synthetic 10xHis tag
HHHHHHHHHH

SEQ ID NO: 21 - protein, 12 aa, Artificial Sequence
  Description of Artificial Sequence Synthetic peptide
GVAMPGAEDDVV

SEQ ID NO: 22 - protein, 8 aa, Artificial Sequence
  Description of Artificial Sequence Synthetic peptide
DYKDDDDK

SEQ ID NO: 23 - protein, 9 aa, Artificial Sequence
  Description of Artificial Sequence Synthetic peptide
YPYDVPDYA

SEQ ID NO: 24 - protein, 10 aa, Artificial Sequence
  Description of Artificial Sequence Synthetic peptide
EQKLISEEDL

SEQ ID NO: 25 - protein, 6 aa, Artificial Sequence
  Description of Artificial Sequence Synthetic peptide
LVPRGS

SEQ ID NO: 26 - protein, 7 aa, Artificial Sequence
  Description of Artificial Sequence Synthetic peptide
  MOD_RES (7)..(7): Gly or Ser
ENLYFQX

SEQ ID NO: 27 - protein, 5 aa, Artificial Sequence
  Description of Artificial Sequence Synthetic peptide
DDDDK

SEQ ID NO: 28 - protein, 6 aa, Artificial Sequence
  Description of Artificial Sequence Synthetic peptide
GGGGGG

SEQ ID NO: 29 - protein, 9 aa, Artificial Sequence
  Description of Artificial Sequence Synthetic peptide
GGGGAGGGG

SEQ ID NO: 30 - protein, 21 aa, Thosea asigna virus
  MISC_FEATURE (1)..(3): May or may not be present
GSGEGRGSLLTCGDVEENPGP

SEQ ID NO: 31 - protein, 22 aa, Porcine teschovirus-1
  MISC_FEATURE (1)..(3): May or may not be present
GSGATNFSLLKQAGDVEENPGP

SEQ ID NO: 32 - protein, 23 aa, Equine rhinitis A virus
  MISC_FEATURE (1)..(3): May or may not be present
GSGQCTNYALLKLAGDVESNPGP

SEQ ID NO: 33 - protein, 25 aa, Foot-and-mouth disease virus
  MISC_FEATURE (1)..(3): May or may not be present
GSGVKQTLNFDLLKLAGDVESNPGP